SEQUENCE LISTING (76 sequences)

SEQ ID NO: 1 - DNA, 17 bp, Artificial Sequence
  source: /note=“Description of Artificial Sequence Synthetic oligonucleotide”
taatacgactcactata

SEQ ID NO: 2 - DNA, 18 bp, Artificial Sequence
  source: /note=“Description of Artificial Sequence Synthetic oligonucleotide”
aattaaccctcactaaag

SEQ ID NO: 3 - DNA, 18 bp, Artificial Sequence
  source: /note=“Description of Artificial Sequence Synthetic oligonucleotide”
atttaggtgacactatag

SEQ ID NO: 4 - DNA, 15 bp, Artificial Sequence
  source: /note=“Description of Artificial Sequence Synthetic oligonucleotide”
attgggcacccgtaa

SEQ ID NO: 5 - DNA, 80 bp, Artificial Sequence
  source: /note=“Description of Artificial Sequence Synthetic oligonucleotide”
gttttagagctagaaatagcaagttaaaataaggctagtccgttatcaacttgaaaaagtggcaccgagtcggtgctttt

SEQ ID NO: 6 - RNA, 107 nt, Artificial Sequence
  source: /note=“Description of Artificial Sequence Synthetic polynucleotide”
  modified_base (4)..(28): a, c, u, g, unknown or other
gggnnnnnnnnnnnnnnnnnnnnnnnnnguuuuagagcuagaaauagcaaguuaaaauaaggcuaguccguuaucaacuugaaaaaguggcaccgagucgguggugc

SEQ ID NO: 7 - RNA, 95 nt, Artificial Sequence
  source: /note=“Description of Artificial Sequence Synthetic oligonucleotide”
  modified_base (1)..(19): a, c, u, g, unknown or other
nnnnnnnnnnnnnnnnnnnguuuuagagcuagaaauagcaaguuaaaauaaggcuaguccguuaucaacuugaaaaaguggcaccgagucggugc

SEQ ID NO: 8 - DNA, 19 bp, Artificial Sequence
  source: /note=“Description of Artificial Sequence Synthetic oligonucleotide”
gctagttattgctcagcgg

SEQ ID NO: 9 - DNA, 85 bp, Artificial Sequence
  source: /note=“Description of Artificial Sequence Synthetic oligonucleotide”
ggccggcatggtcccagcctcctcgctggcgccggctgggcaacattccgaggggaccgtcccctcggtaatggcgaatgggacg

SEQ ID NO: 10 - DNA, 22 bp, Artificial Sequence
  source: /note=“Description of Artificial Sequence Synthetic oligonucleotide”
ggatccggaggccggagaattg

SEQ ID NO: 11 - DNA, 139 bp, Artificial Sequence
  source: /note=“Description of Artificial Sequence Synthetic polynucleotide”
aaaagcaccgactcggtgccactttttcaagttgataacggactagccttattttaacttgctatttctagctctaaaactgaagaagatggtgcgctcctatagtgagtcgtattacaattctccggcctccggatcc

SEQ ID NO: 12 - DNA, 139 bp, Artificial Sequence; identical to SEQ ID NO: 11
  source: /note=“Description of Artificial Sequence Synthetic polynucleotide”

SEQ ID NO: 13 - DNA, 139 bp, Artificial Sequence
  source: /note=“Description of Artificial Sequence Synthetic polynucleotide”
ggatccggaggccggagaattgtaatacgactcactataggagcgcaccatcttcttcagttttagagctagaaatagcaagttaaaataaggctagtccgttatcaacttgaaaaagtggcaccgagtcggtgctttt

SEQ ID NO: 14 - DNA, 135 bp, Artificial Sequence
  source: /note=“Description of Artificial Sequence Synthetic polynucleotide”
ggatccggaggccggagaattgtaatacgactcactataggagcgcaccatcttcttcagttttagagctagaaatagcaagttaaaataaggctagtccgttatcaacttgaaaaagtggcaccgagtcggtgc

SEQ ID NO: 15 - DNA, 139 bp, Artificial Sequence; identical to SEQ ID NO: 11
  source: /note=“Description of Artificial Sequence Synthetic polynucleotide”

SEQ ID NO: 16 - DNA, 20 bp, Artificial Sequence
  source: /note=“Description of Artificial Sequence Synthetic oligonucleotide”
ggagcgcaccatcttcttca

SEQ ID NO: 17 - RNA, 100 nt, Artificial Sequence
  source: /note=“Description of Artificial Sequence Synthetic polynucleotide”
aucagaggccaaacccuuccguuuuagagcuagaaauagcaaguuaaaauaaggcuaguccguuaucaacuugaaaaaguggcaccgagucggugcuuuu

SEQ ID NO: 18 - RNA, 100 nt, Artificial Sequence
  source: /note=“Description of Artificial Sequence Synthetic polynucleotide”
acugaaucggaacaaggcaaguuuuagagcuagaaauagcaaguuaaaauaaggcuaguccguuaucaacuugaaaaaguggcaccgagucggugcuuuu

SEQ ID NO: 19 - DNA, 18 bp, Artificial Sequence
  source: /note=“Description of Artificial Sequence Synthetic oligonucleotide”
taatacgactcactatag

SEQ ID NO: 20 - DNA, 18 bp, Artificial Sequence
  source: /note=“Description of Artificial Sequence Synthetic oligonucleotide”
taatacgactcactataa

SEQ ID NO: 21 - DNA, 22 bp, Artificial Sequence
  source: /note=“Description of Artificial Sequence Synthetic primer”
aaaagcaccgactcggtgccac

SEQ ID NO: 22 - DNA, 25 bp, Artificial Sequence
  source: /note=“Description of Artificial Sequence Synthetic primer”
taacgccagggttttcccagtcacg

SEQ ID NO: 23 - DNA, 83 bp, Artificial Sequence
  source: /note=“Description of Artificial Sequence Synthetic primer”
aggaaacagctatgaccatgctcgagccaagctcggcgcgccattgggatggaacgaagacccaaaagcaccgactcggtgcc

SEQ ID NO: 24 - DNA, 83 bp, Artificial Sequence; identical to SEQ ID NO: 23
  source: /note=“Description of Artificial Sequence Synthetic primer”

SEQ ID NO: 25 - DNA, 20 bp, Artificial Sequence
  source: /note=“Description of Artificial Sequence Synthetic primer”
aaaagcaccgactcggtgcc

SEQ ID NO: 26 - DNA, 24 bp, Artificial Sequence
  source: /note=“Description of Artificial Sequence Synthetic primer”
gtatgttgtgtggaattgtgagcg

SEQ ID NO: 27 - DNA, 17 bp, Artificial Sequence
  source: /note=“Description of Artificial Sequence Synthetic oligonucleotide”
taatacgactcactatt

SEQ ID NO: 28 - DNA, 17 bp, Artificial Sequence
  source: /note=“Description of Artificial Sequence Synthetic oligonucleotide”
taatacgactcacaatc

SEQ ID NO: 29 - DNA, 17 bp, Artificial Sequence
  source: /note=“Description of Artificial Sequence Synthetic oligonucleotide”
taatacgactcactccg

SEQ ID NO: 30 - DNA, 17 bp, Artificial Sequence
  source: /note=“Description of Artificial Sequence Synthetic oligonucleotide”
tactacgactcactata

SEQ ID NO: 31 - DNA, 22 bp, Artificial Sequence
  source: /note=“Description of Artificial Sequence Synthetic primer”
agcgcccaatacgcaaaccgcc

SEQ ID NO: 32 - DNA, 22 bp, Artificial Sequence
  source: /note=“Description of Artificial Sequence Synthetic primer”
gcgttggccgattcattaatgc

SEQ ID NO: 33 - RNA, 114 nt, Artificial Sequence
  source: /note=“Description of Artificial Sequence Synthetic polynucleotide”
  modified_base (1)..(25): a, c, u, g, unknown or other
  misc_feature (1)..(25): /note=“This region may encompass 15-25 nucleotides”
nnnnnnnnnnnnnnnnnnnnnnnnnguuuuagagcuaugcuggaaacagcauagcaaguuaaaauaaggcuaguccguuaucaacuugaaaaaguggcaccgagucggugcuuu

SEQ ID NO: 34 - RNA, 146 nt, Artificial Sequence
  source: /note=“Description of Artificial Sequence Synthetic polynucleotide”
  modified_base (1)..(25): a, c, u, g, unknown or other
  misc_feature (1)..(25): /note=“This region may encompass 15-25 nucleotides”
nnnnnnnnnnnnnnnnnnnnnnnnnguuguagcucccuuucucauuucggaaacgaaaugagaaccguugcuacaauaaggccgucugaaaagaugugccgcaacgcucugccccuuaaagcuucugcuuuaaggggcaucguuua

SEQ ID NO: 35 - RNA, 116 nt, Artificial Sequence
  source: /note=“Description of Artificial Sequence Synthetic polynucleotide”
  modified_base (1)..(25): a, c, u, g, unknown or other
  misc_feature (1)..(25): /note=“This region may encompass 15-25 nucleotides”
nnnnnnnnnnnnnnnnnnnnnnnnnguuuuaguacucuguaauuugaaaaaauuacagaaucuacuaaaacaaggcaaaaugccguguuuaucucgucaacuuguuggcgagauuu

SEQ ID NO: 36 - RNA, 121 nt, Artificial Sequence
  source: /note=“Description of Artificial Sequence Synthetic polynucleotide”
  modified_base (1)..(25): a, c, u, g, unknown or other
  misc_feature (1)..(25): /note=“This region may encompass 15-25 nucleotides”
nnnnnnnnnnnnnnnnnnnnnnnnnguuuuuguacucucaagauucaauaaucuugcagaagcuacaaagauaaggcuucaugccgaaaucaacacccugucauuuuauggcaggguguuu

SEQ ID NO: 37 - DNA, 120 bp, Artificial Sequence
  source: /note=“Description of Artificial Sequence Synthetic oligonucleotide”
  source: /note=“Description of Combined DNA/RNA Molecule Synthetic oligonucleotide”
  modified_base (1)..(25): a, c, t, g, unknown or other
  misc_feature (1)..(25): /note=“This region may encompass 15-25 nucleotides”
nnnnnnnnnnnnnnnnnnnnnnnnnguuuuagagcuguguuguuugttaaaacaacacagcgaguuaaaauaaggcuuaguccguacucaacuugaaaagguggcaccgauucgguguuu

SEQ ID NO: 38 - RNA, 101 nt, Artificial Sequence
  source: /note=“Description of Artificial Sequence Synthetic polynucleotide”
  modified_base (1)..(25): a, c, u, g, unknown or other
  misc_feature (1)..(25): /note=“This region may encompass 15-25 nucleotides”
nnnnnnnnnnnnnnnnnnnnnnnnnguuuuagucccugaaaagggacuaaaauaaagaguuugcgggacucugcgggguuacaauccccuaaaaccgcuuu

SEQ ID NO: 39 - RNA, 143 nt, Artificial Sequence
  source: /note=“Description of Artificial Sequence Synthetic polynucleotide”
  modified_base (1)..(25): a, c, u, g, unknown or other
  misc_feature (1)..(25): /note=“This region may encompass 15-25 nucleotides”
nnnnnnnnnnnnnnnnnnnnnnnnngucauaguuccccugagaaaucaggguuacuaugauaagggcuuucugccuaaggcagacugacccgcggcguuggggaucgccugucgcccgcuuuuggcgggcauuccccauccuu

SEQ ID NO: 40 - RNA, 98 nt, Artificial Sequence
  source: /note=“Description of Artificial Sequence Synthetic oligonucleotide”
  modified_base (1)..(25): a, c, u, g, unknown or other
  misc_feature (1)..(25): /note=“This region may encompass 15-25 nucleotides”
nnnnnnnnnnnnnnnnnnnnnnnnnguuucaguugcgccgaaaggcgcucuguaaucauuuaaaaguauuuugaacggaccucuguuugacacgucug

SEQ ID NO: 41 - RNA, 45 nt, Artificial Sequence
  source: /note=“Description of Artificial Sequence Synthetic oligonucleotide”
  modified_base (21)..(45): a, c, u, g, unknown or other
  misc_feature (21)..(45): /note=“This region may encompass 15-25 nucleotides”
uaauuucuacuguuguagaunnnnnnnnnnnnnnnnnnnnnnnnn

SEQ ID NO: 42 - RNA, 45 nt, Artificial Sequence
  source: /note=“Description of Artificial Sequence Synthetic oligonucleotide”
  modified_base (21)..(45): a, c, u, g, unknown or other
  misc_feature (21)..(45): /note=“This region may encompass 15-25 nucleotides”
uaauuucuacucuuguagaunnnnnnnnnnnnnnnnnnnnnnnnn

SEQ ID NO: 43 - RNA, 45 nt, Artificial Sequence
  source: /note=“Description of Artificial Sequence Synthetic oligonucleotide”
  modified_base (21)..(45): a, c, u, g, unknown or other
  misc_feature (21)..(45): /note=“This region may encompass 15-25 nucleotides”
uaauuucuacuauuguagaunnnnnnnnnnnnnnnnnnnnnnnnn

SEQ ID NO: 44 - RNA, 46 nt, Artificial Sequence
  source: /note=“Description of Artificial Sequence Synthetic oligonucleotide”
  modified_base (22)..(46): a, c, u, g, unknown or other
  misc_feature (22)..(46): /note=“This region may encompass 15-25 nucleotides”
uaauuucuacucuuuguagaunnnnnnnnnnnnnnnnnnnnnnnnn

SEQ ID NO: 45 - RNA, 44 nt, Artificial Sequence
  source: /note=“Description of Artificial Sequence Synthetic oligonucleotide”
  modified_base (20)..(44): a, c, u, g, unknown or other
  misc_feature (20)..(44): /note=“This region may encompass 15-25 nucleotides”
uaauuucuacuuuguagaunnnnnnnnnnnnnnnnnnnnnnnnn

SEQ ID NO: 46 - RNA, 46 nt, Artificial Sequence
  source: /note=“Description of Artificial Sequence Synthetic oligonucleotide”
  modified_base (22)..(46): a, c, u, g, unknown or other
  misc_feature (22)..(46): /note=“This region may encompass 15-25 nucleotides”
uaauuucuacuguuuguagaunnnnnnnnnnnnnnnnnnnnnnnnn

SEQ ID NO: 47 - RNA, 45 nt, Artificial Sequence
  source: /note=“Description of Artificial Sequence Synthetic oligonucleotide”
  modified_base (21)..(45): a, c, u, g, unknown or other
  misc_feature (21)..(45): /note=“This region may encompass 15-25 nucleotides”
uaauuucuacuucgguagaunnnnnnnnnnnnnnnnnnnnnnnnn

SEQ ID NO: 48 - RNA, 117 nt, Artificial Sequence
  source: /note=“Description of Artificial Sequence Synthetic polynucleotide”
  modified_base (93)..(117): a, c, u, g, unknown or other
  misc_feature (93)..(117): /note=“This region may encompass 15-25 nucleotides”
ggucuagaggacagaauuuuucaacgggugugccaauggccacuuuccagguggcaaagcccguugagcuucucaaaucugagaaguggcacnnnnnnnnnnnnnnnnnnnnnnnnn

SEQ ID NO: 49 - RNA, 55 nt, Artificial Sequence
  source: /note=“Description of Artificial Sequence Synthetic oligonucleotide”
  modified_base (31)..(55): a, c, u, g, unknown or other
  misc_feature (31)..(55): /note=“This region may encompass 15-25 nucleotides”
ggccaccccaauaucgaaggggacuaaaacnnnnnnnnnnnnnnnnnnnnnnnnn

SEQ ID NO: 50 - RNA, 43 nt, Artificial Sequence
  source: /note=“Description of Artificial Sequence Synthetic oligonucleotide”
  modified_base (19)..(43): a, c, u, g, unknown or other
  misc_feature (19)..(43): /note=“This region may encompass 15-25 nucleotides”
aauucuacucuuguagaunnnnnnnnnnnnnnnnnnnnnnnnn

SEQ ID NO: 51 - RNA, 61 nt, Artificial Sequence
  source: /note=“Description of Artificial Sequence Synthetic oligonucleotide”
  modified_base (1)..(25): a, c, u, g, unknown or other
  misc_feature (1)..(25): /note=“This region may encompass 15-25 nucleotides”
nnnnnnnnnnnnnnnnnnnnnnnnnguuguggaagguccaguuuugggggcuauuacaaca

SEQ ID NO: 52 - RNA, 76 nt, Artificial Sequence
  source: /note=“Description of Artificial Sequence Synthetic oligonucleotide”
  source: /note=“See specification as filed for detailed description of substitutions and preferred embodiments”
guuuuagagcuagaaauagcaaguuaaaauaaggcuaguccguuaucaacuugaaaaaguggcaccgagucggugc

SEQ ID NO: 53 - RNA, 76 nt, Artificial Sequence
  source: /note=“Description of Artificial Sequence Synthetic oligonucleotide”
  source: /note=“See specification as filed for detailed description of substitutions and preferred embodiments”
guuuaagagcuagaaauagcaaguuuaaauaaggcuaguccguuaucaacuugaaaaaguggcaccgagucggugc

SEQ ID NO: 54 - RNA, 86 nt, Artificial Sequence
  source: /note=“Description of Artificial Sequence Synthetic oligonucleotide”
  source: /note=“See specification as filed for detailed description of substitutions and preferred embodiments”
guuuuagagcuaugcuggaaacagcauagcaaguuaaaauaaggcuaguccguuaucaacuugaaaaaguggcaccgagucggugc

SEQ ID NO: 55 - RNA, 86 nt, Artificial Sequence
  source: /note=“Description of Artificial Sequence Synthetic oligonucleotide”
  source: /note=“See specification as filed for detailed description of substitutions and preferred embodiments”
guuuaagagcuaugcuggaaacagcauagcaaguuuaaauaaggcuaguccguuaucaacuugaaaaaguggcaccgagucggugc

SEQ ID NO: 56 - RNA, 80 nt, Artificial Sequence
  source: /note=“Description of Artificial Sequence Synthetic oligonucleotide”
guuuuagagcuagaaauagcaaguuaaaauaaggcuaguccguuaucaacuugaaaaaguggcaccgagucggugcuuuu

SEQ ID NO: 57 - RNA, 90 nt, Artificial Sequence
  source: /note=“Description of Artificial Sequence Synthetic oligonucleotide”
guuuaagagcuaugcuggaaacagcauagcaaguuuaaauaaggcuaguccguuaucaacuugaaaaaguggcaccgagucggugcuuuu

SEQ ID NO: 58 - RNA, 100 nt, Artificial Sequence
  source: /note=“Description of Artificial Sequence Synthetic polynucleotide”
  modified_base (1)..(20): a, c, u, g, unknown or other
nnnnnnnnnnnnnnnnnnnnguuuuagagcuagaaauagcaaguuaaaauaaggcuaguccguuaucaacuugaaaaaguggcaccgagucggugcuuuu

SEQ ID NO: 59 - RNA, 12 nt, Artificial Sequence
  source: /note=“Description of Artificial Sequence Synthetic oligonucleotide”
guuuuagagcua

SEQ ID NO: 60 - RNA, 12 nt, Artificial Sequence
  source: /note=“Description of Artificial Sequence Synthetic oligonucleotide”
guuuaagagcua

SEQ ID NO: 61 - RNA, 17 nt, Artificial Sequence
  source: /note=“Description of Artificial Sequence Synthetic oligonucleotide”
guuuuagagcuaugcug

SEQ ID NO: 62 - RNA, 17 nt, Artificial Sequence
  source: /note=“Description of Artificial Sequence Synthetic oligonucleotide”
guuuaagagcuaugcug

SEQ ID NO: 63 - RNA, 22 nt, Artificial Sequence
  source: /note=“Description of Artificial Sequence Synthetic oligonucleotide”
guuuuagagcuaugcuguuuug

SEQ ID NO: 64 - RNA, 22 nt, Artificial Sequence
  source: /note=“Description of Artificial Sequence Synthetic oligonucleotide”
guuuaagagcuaugcuguuuug

SEQ ID NO: 65 - RNA, 16 nt, Artificial Sequence
  source: /note=“Description of Artificial Sequence Synthetic oligonucleotide”
guuuuagagcuaugcu

SEQ ID NO: 66 - RNA, 60 nt, Artificial Sequence
  source: /note=“Description of Artificial Sequence Synthetic oligonucleotide”
  source: /note=“See specification as filed for detailed description of substitutions and preferred embodiments”
uagcaaguuaaaauaaggcuaguccguuaucaacuugaaaaaguggcaccgagucggugc

SEQ ID NO: 67 - RNA, 60 nt, Artificial Sequence
  source: /note=“Description of Artificial Sequence Synthetic oligonucleotide”
  source: /note=“See specification as filed for detailed description of substitutions and preferred embodiments”
uagcaaguuuaaauaaggcuaguccguuaucaacuugaaaaaguggcaccgagucggugc

SEQ ID NO: 68 - RNA, 65 nt, Artificial Sequence
  source: /note=“Description of Artificial Sequence Synthetic oligonucleotide”
  source: /note=“See specification as filed for detailed description of substitutions and preferred embodiments”
cagcauagcaaguuaaaauaaggcuaguccguuaucaacuugaaaaaguggcaccgagucggugc

SEQ ID NO: 69 - RNA, 65 nt, Artificial Sequence
  source: /note=“Description of Artificial Sequence Synthetic oligonucleotide”
  source: /note=“See specification as filed for detailed description of substitutions and preferred embodiments”
cagcauagcaaguuuaaauaaggcuaguccguuaucaacuugaaaaaguggcaccgagucggugc

SEQ ID NO: 70 - RNA, 74 nt, Artificial Sequence
  source: /note=“Description of Artificial Sequence Synthetic oligonucleotide”
  source: /note=“See specification as filed for detailed description of substitutions and preferred embodiments”
aacagcauagcaaguuaaaauaaggcuaguccguuaucaacuugaaaaaguggcaccgagucggugcuuuuuuu

SEQ ID NO: 71 - RNA, 74 nt, Artificial Sequence
  source: /note=“Description of Artificial Sequence Synthetic oligonucleotide”
  source: /note=“See specification as filed for detailed description of substitutions and preferred embodiments”
aacagcauagcaaguuuaaauaaggcuaguccguuaucaacuugaaaaaguggcaccgagucggugcuuuuuuu

SEQ ID NO: 72 - RNA, 67 nt, Artificial Sequence
  source: /note=“Description of Artificial Sequence Synthetic oligonucleotide”
  source: /note=“See specification as filed for detailed description of substitutions and preferred embodiments”
aacagcauagcaaguuaaaauaaggcuaguccguuaucaacuugaaaaaguggcaccgagucggugc

SEQ ID NO: 73 - RNA, 90 nt, Artificial Sequence; identical to SEQ ID NO: 57
  source: /note=“Description of Artificial Sequence Synthetic oligonucleotide”
  source: /note=“See specification as filed for detailed description of substitutions and preferred embodiments”

SEQ ID NO: 74 - RNA, 67 nt, Artificial Sequence
  source: /note=“Description of Artificial Sequence Synthetic oligonucleotide”
  source: /note=“See specification as filed for detailed description of substitutions and preferred embodiments”
agcauagcaaguuaaaauaaggcuaguccguuaucaacuugaaaaaguggcaccgagucggugcuuu

SEQ ID NO: 75 - RNA, 85 nt, Artificial Sequence
  source: /note=“Description of Artificial Sequence Synthetic oligonucleotide”
  source: /note=“See specification as filed for detailed description of substitutions and preferred embodiments”
guuggaaccauucaaaacagcauagcaaguuaaaauaaggcuaguccguuaucaacuugaaaaaguggcaccgagucggugcuuu

SEQ ID NO: 76 - RNA, 100 nt, Artificial Sequence
  source: /note=“Description of Artificial Sequence Synthetic polynucleotide”
ggccgagaugucucgcuccgguuuuagagcuagaaauagcaaguuaaaauaaggcuaguccguuaucaacuugaaaaaguggcaccgagucggugcuuuu

(19) United States
(12) Patent Application Publication
(10) Pub. No.: US 2021/0180053 A1
(43) Pub. Date: Jun. 17, 2021

(54) SYNTHETIC RNAS AND METHODS OF USE

(71) Applicant: Novartis AG, Basel (CH)

(72) Inventors: Michael BEVERLY, Lexington, MA (US); Caitlin Jeanette HAGEN, Cambridge, MA (US); Olga SLACK, Newton, MA (US); Jan WEILER, Newton, MA (US)

(21) Appl. No.: 16/760,897

(22) PCT Filed: Oct. 31, 2018
(86) PCT No.: PCT/IB2018/058562

§ 371 Date: Apr. 30, 2020

Related U.S. Application Data
(60) Provisional application No. 62/579,979, filed on Nov. 1, 2017.

(51) Int. Cl. C12N 15/11; C12P 19/34; C12N 9/22; C12N 15/90

(52) U.S. Cl. CPC C12N 15/11; C12P 19/34; C12N 9/22; C12N 15/907; C12N 2310/3341; C12N 2800/00; C12N 2310/317; C12N 2310/321; C12N 2310/20

(57) ABSTRACT

A manufacturing process of RNA having a length of about 20-200 bases with improved performance, by using in vitro transcription in combination with other methodologies that may increase yield and quality. A manufacturing process of RNA having a length of about 2-200 bases with improved performance

### CROSS-REFERENCE TO RELATED APPLICATION

This application claims the priority and the benefit of U.S. Patent Application No. 62/579,979, filed Nov. 1, 2017, the contents of which are incorporated herein by their entireties.

### REFERENCE TO A “SEQUENCE LISTING,” A TABLE, OR A COMPUTER PROGRAM LISTING APPENDIX SUBMITTED AS AN ASCII FILE

The Sequence Listing written in file PAT057679-WO-PCT_SL.TXT, created Oct. 29, 2018, 29,326 bytes in size, machine format IBM-PC, MS-Windows operating system, is hereby incorporated by reference.

### FIELD OF THE INVENTION

The invention relates generally a process of using an enzyme to synthesize nucleic acids, particularly to in vitro transcription, and, e.g., to the in vitro transcription of guide RNAs for use in Clustered Regularly Interspaced Short Palindromic Repeats (CRISPR) technologies.

### BACKGROUND OF THE INVENTION

A CRISPR (Clustered Regularly Interspaced Short Palindromic Repeats) system is a combination of protein and ribonucleic acid (“RNA”) that can alter the genetic sequence of an organism. In their natural environments, CRISPR systems protect bacteria against infection by viruses. CRISPR systems are now being developed as powerful tools to modify specific deoxyribonucleic acid (DNA) sequences in the genomes of other organisms, from plants to animals.

A Type II CRISPR-Cas system comprises three components: (1) a CRISPR RNA (crRNA) molecule, which is also called a “guide sequence” in PCT patent publication WO 2014/093661 (The Broad Institute, Inc., Massachusetts Institute of Technology) and a “targeter-RNA” in WO 2013/176772 A1 (The Regents of the University of California, University of Vienna, Jennifer A. Doudna); (2) a trans-activating crRNA (tracrRNA), which is called an “activator-RNA” in WO 2013/176772 A1, (3) and a nuclease or other effector protein, for example, protein called Cas9 (formerly CSN1). The crRNA and the tracrRNA can be joined as a single polynucleotide known as a single guide RNA (sgRNA). To alter a DNA molecule, a Type 11 CRISPR-Cas system achieves three interactions: (1) crRNA binding by specific base pairing to a specific sequence in the DNA of interest (target DNA); (2) crRNA binding by specific base pairing at another sequence to a tracrRNA; and (3) portions of the gRNA interacting with a Cas9 protein, which then cuts the target DNA at the specific site. These interactions are illustrated in FIG. 2 of JENNIFER A. DOUDNA, EMMANUELLE CHARPENTIER SCIENCE 28 Nov. 2014, which shows a double-stranded target DNA sequence that is bound to a crRNA (as indicated by the vertical black lines showing nucleic acid base pairing). A different part of the crRNA is bound to a tracrRNA. The tracrRNA interacts with a Cas9 protein that cuts the target DNA in a site-specific matter. By linking a DNA-cutting enzyme to a specific site on the target DNA, the CRISPR-Cas9 system achieves specific, targeted manipulation of DNA.

Because of the power of CRISPR systems as biotechnological methods, use of CRISPR systems is expected to grow. A problem with this growth is that there is currently not a satisfactory method for large-scale production of high-quality sgRNA. Current solid-phase chemical synthesis methods are not expected to meet the demand, for several reasons described in the specification below.

Thus, there is a need in the biotechnological art for a method for large-scale production of high-quality RNA molecules, for example, mRNA fragments, interfering RNAs, RNA aptamers, gRNAs, such as for example, sgRNA.

### SUMMARY OF THE INVENTION

Provided herein is a DNA template (an IVT cassette) for making a ribonucleic acid (RNA) transcript having a length of about 20-200 bases, where the DNA template includes (a) a first deoxyribonucleic acid (DNA) sequence comprising a RNA transcription initiation site; (b) a polymerase promoter upstream from the RNA transcription initiation site; (c) a second DNA sequence encoding the RNA transcript having a length of about 20-200 bases disposed downstream of the RNA transcription initiation site; and (d) a linearization site downstream from the RNA transcription initiation site.

In some embodiments, the DNA template is part of a DNA plasmid.

In some embodiments, the polymerase promoter is selected from the group consisting of T7 polymerase promoter, a T3 polymerase promoter, an SP6 polymerase promoter, a Syn5 polymerase promoter, and an E. coli RNase promoter.

In some embodiments, the linearization site is a restriction endonuclease site.

In some embodiments, the restriction endonuclease site is selected from the group consisting of DraI, BspQI, SapI and BbsI.

In some embodiments, the DNA template has been linearized.

In some embodiments, the DNA template further includes a ribozyme sequence, e.g., downstream from the RNA transcription initiation site and upstream of the linearization site.

In some embodiments, the ribozyme sequence is selected from the group consisting of hammerhead, hairpin, hepatitis delta virus and Varkud satellite ribozyme.

In some embodiments, the DNA template further includes a T7 terminator sequence, e.g., downstream from the RNA transcription initiation site and upstream of the linearization site.

In some embodiments, the DNA template further includes a promoter enhancing sequence upstream from the RNA transcription initiation site.

In some embodiments, RNA transcript having a length of about 20-200 bases comprises a single guide RNA (sgRNA) sequence.

In some embodiments, the sgRNA sequence is about 50 bases to 150 bases in length.

Also provided herein is a double stranded DNA (dsDNA) template for making a ribonucleic acid (RNA) transcript having a length of about 20-200 bases, where the dsDNA template includes (a) a first DNA sequence comprising an RNA transcription initiation site; (b) a polymerase promoter upstream from the RNA transcription initiation site, (c) a second DNA sequence encoding the RNA transcript having a length of about 20-200 bases disposed downstream of the RNA transcription initiation site; and (d) one or more modified nucleotides at the 5′ end of the antisense strand of the dsDNA template.

In some embodiments, the dsDNA template includes a transcriptional enhancer sequence upstream of the polymerase promoter.

In some embodiments, the modified nucleotide comprises 2′-O-alkyl modification.

In some embodiments, the modified nucleotide is 2′-O-methyl modified nucleotide or 2′-O-(2-methoxyethyl) modified nucleotide.

In some embodiments, the polymerase promoter is selected from the group consisting of T7 polymerase promoter, a T3 polymerase promoter, an SP6 polymerase promoter, a Syn5 polymerase promoter, and an E. coli RNase promoter.

In some embodiments, the linearization site is a restriction endonuclease site.

In some embodiments, the restriction endonuclease site is selected from the group consisting of DraI, BspQI, SapI and BbsI.

In some embodiments, the RNA transcript having a length of about 20-200 bases comprises a sgRNA sequence.

In some embodiments, the sgRNA sequence is about 50 bases to 150 bases in length.

Further provided herein is a partially single stranded DNA (ssDNA) template for making a ribonucleic acid (RNA) transcript having a length of about 20-200 bases, where the ssDNA template includes (a) a first DNA sequence comprising an RNA transcription initiation site; (b) a polymerase promoter upstream from the RNA transcription initiation site, (c) a second DNA sequence encoding the RNA transcript having a length of about 20-200 bases disposed downstream of the RNA transcription initiation site; and (d) one or more modified nucleotides at the 5′ end of the antisense strand of the dsDNA template.

In some embodiments, the partially ssDNA template includes a transcriptional enhancer sequence upstream of the polymerase promoter.

In some embodiments, the modified nucleotide comprises 2′-O-alkyl modification.

In some embodiments, the modified nucleotide is 2′-O-methyl modified nucleotide or 2′-O-(2-methoxyethyl) modified nucleotide.

In some embodiments, the single stranded DNA is complementary to all or a portion of the polymerase promoter.

In some embodiments, the polymerase promoter is selected from the group consisting of T7 polymerase promoter, a T3 polymerase promoter, an SP6 polymerase promoter, a Syn5 polymerase promoter, and an E. coli RNase promoter.

In some embodiments, the RNA transcript having a length of about 20-200 bases comprises a sgRNA sequence.

In some embodiments, the sgRNA sequence is about 50 bases to 150 bases in length.

Also provided herein is a method of making a ribonucleic acid (RNA) having a length of about 20-200 bases by in vitro transcription (IVT), including the steps of (a) obtaining a DNA template described herein, and (b) making the RNA transcript by in vitro transcription.

In some embodiments, the method includes the step of amplifying the DNA template using PCR.

In some embodiments, the method further includes the step of purifying the produced RNA transcript by reverse-phase chromatography.

In some embodiments, the method further includes the step of testing the purified produced RNA transcript for the presence of immune stimulating moieties by an immunogenicity assay.

In some embodiments, the produced RNA transcript is substantially free of any immune stimulating moieties.

In some embodiments, the produced RNA transcript is substantially free of n+x variants (e.g., where X=1).

In some embodiments, the produced RNA transcript is substantially free of n−x variants (e.g., where X=1).

In some embodiments, the RNA transcript comprises a sgRNA.

In some embodiments, the sgRNA is about 50 bases to 150 bases in length.

Also provided herein is a composition including a ribonucleic acid (RNA) transcript having a length of about 20-200 bases, made by the process described herein, where (a) the composition comprising the RNA transcript is substantially free of immune stimulating moieties, and/or (b) the composition is substantially free of RNA transcripts having n−1 variants and/or n+1 variants.

In some embodiments, the RNA comprises pseudouridine (ψ), or 5-methylcytidine (m⁵C), or both ψ and m⁵C.

In some embodiments, the RNA transcript in the composition is about 50 bases to 150 bases in length.

In some embodiments, the RNA transcript is dephosphorylated or capped at the 5′ end, at the 3′ end, or at the 5′ and 3′ ends.

In some embodiments, the RNA transcript comprises a sgRNA transcript.

Also provided herein is a pharmaceutical composition, including the composition described herein, and a pharmaceutically acceptable carrier.

Further provided herein is a composition including an IVT-made polynucleotide having a length of about 20-200 bases, where the composition is substantially free of immune stimulating moieties and/or is substantially free of n−1 or n+1 variants.

In some embodiments, the IVT-made polynucleotide comprises pseudouridine (ψ), or 5-methylcytidine (m⁵C), or both ψ and m⁵C.

In some embodiments, the IVT-made polynucleotide is about 50 bases to 150 bases in length.

In some embodiments, the IVT-made polynucleotide is dephosphorylated or capped at the 5′ end, at the 3′ end, or at the 5′ and 3′ ends.

In some embodiments, the IVT-made polynucleotide is a sgRNA sequence.

In some embodiments, the sgRNA sequence is about 50 bases to 150 bases in length.

Also included herein is a cell comprising a composition or a pharmaceutical composition described herein.

In some embodiments, the cell further includes an RNA-guided DNA endonuclease enzyme.

Also provided herein is a method of altering gene expression in a cell, the method includes introducing into the cell a composition or a pharmaceutical composition described herein.

In some embodiments, the method further includes introducing to the cell an RNA-guided DNA endonuclease enzyme.

In some embodiments, the RNA-guided DNA endonuclease enzyme is Cas9 or Cpf1 or a Class II CRISPR endonuclease or a variant thereof.

In some embodiments, the cell is an animal cell.

In some embodiments, the cell is a mammalian, primate, or human cell.

In some embodiments, the cell is a hematopoietic stem or progenitor cell (HSPC).

Also provided herein is a cell, altered by the method described herein.

Also provided herein is a cell, obtainable by the method described herein.

Also provided herein is the composition or the pharmaceutical composition described herein for use in altering gene expression in a cell.

### BRIEF DESCRIPTION OF THE DRAWINGS

FIG. 1 is a schematic representation of one design of a DNA template for IVT production of sgRNA. The sgRNA sequence is shown as comprising crRNA and optionally tracrRNA elements.

FIG. 2 is a schematic drawing of a plasmid-based template for making a sgRNA.

FIG. 3 is an image of an agarose gel showing electrophoresis of linearized plasmid DNA template and circular plasmid DNA template. The left lane is a molecular weight ladder. The middle lane (1) shows linearized DNA. The right lane (2) shows circular DNA.

FIG. 4 shows a PCR approach to generate a dsDNA template with modified ends for IVT production of sgRNA.

FIG. 5 shows a PCR approach to generate a partially ssDNA template with modified ends for IVT production of sgRNA.

FIG. 6 shows comparison of in vitro transcribed RNA using either natural or chemically modified nucleotides in the sgRNA. Incorporation of pseudouridine (ψ), or combination of pseudouridine (V) and 5-methylcytidine (m⁵C) into the in vitro sgRNA transcript does not affect activity of sgRNA in an in vitro Cas9 assay.

FIG. 7 is a capillary electrophoresis of an in vitro RNA transcript. The left lane is a molecular weight ladder. The right lane (1) shows an in vitro transcript of sgRNA.

FIG. 8 is an image of a gel electrophoresis assay showing the homogeneity of sgRNAs produced by in vitro transcription and by solid-phase chemical synthesis by commercial vendors.

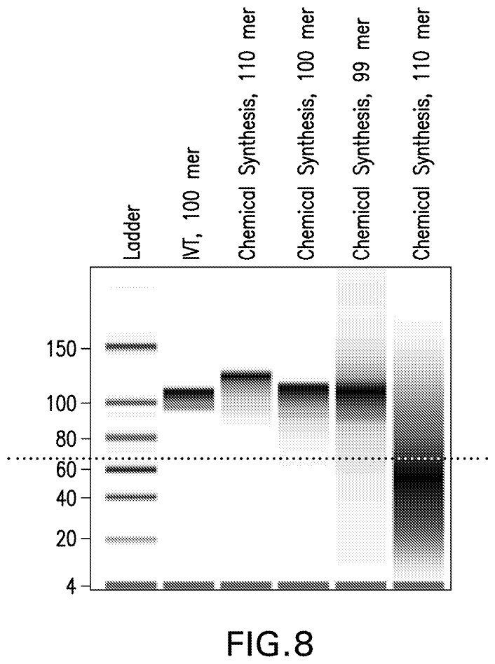

FIG. 9A shows a 100 mer sgRNA produced by in vitro transcription (IVT) from PCR template and measured by LC-MS. The figure shows no n+x entities.

FIG. 9B shows a 100 mer sgRNA produced by in vitro transcription (IVT) from PCR template and measured by LC-MS. The figure shows minor n−x (“N minus”) and n+x (“N plus”) entities.

FIG. 10 shows a 100 mer sgRNA produced by solid-phase chemical synthesis performed by a commercial vendor and measured by LC-MS. The figure shows both n+x entities and n−1 entities, as well as side-products resulting from incomplete deprotection of the chemically synthesized sgRNA product.

FIG. 11 is a gel electrophoresis showing the results of an in vitro Cas9 assay. The figure shows that sgRNA produced by in vitro transcription has comparable activity to sgRNA produced by solid-state chemical synthesis.

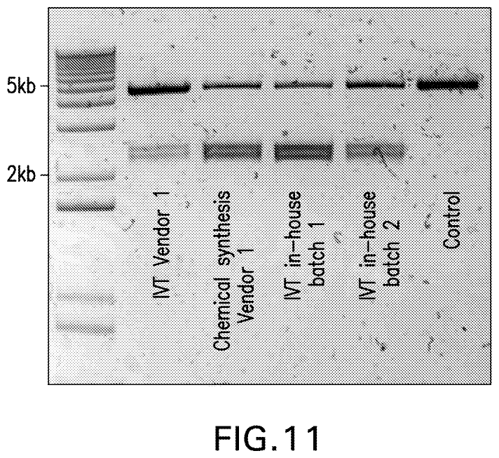

FIG. 12 is a gel-electrophoresis analysis of sgRNA1 and sgRNA2 PCR templates.

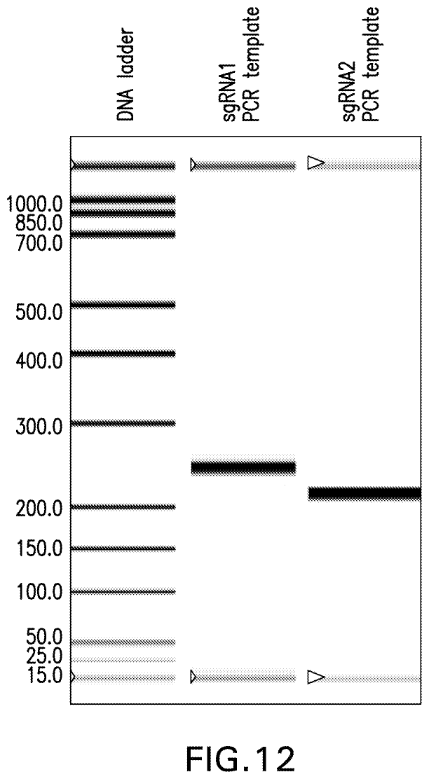

FIG. 13A is an overlapped comparison of chromatograms UV260 nm of IVT product and chemical synthesis product.

FIG. 13B is a chromatograms UV260 nm of IVT product.

FIG. 13C is a chromatograms UV260 nm of chemical synthesis product.

FIG. 14 is a FACS result of a series of transfected cells. MB-CD34 and HSC cells were electroporated with respective sgRNA and cas9 ribonucleoprotein (RNP) and were later harvested and stained with B2M-FITC antibody. FACS analysis was then conducted. Comparison of the Cas9 activity complexed with either chemically synthesized sgRNA3, or IVT-derived sgRNA3 shown. IVT-derived sgRNA3 was also compared as 5′ triphosphate, or 5′ hydroxyl. The results indicated that all sgRNAs prepared via IVT worked either equally well or better than the one that was chemically synthesized.

### DETAILED DESCRIPTION OF THE INVENTION

Each of the patents, patent publications, and patent applications, and all documents cited herein are hereby incorporated herein by reference, and can be used in the practice of the invention.

The recitation of a listing of elements in any definition of a variable herein includes definitions of that variable as any single element, combination or sub-combination of listed elements. The recitation of an embodiment herein includes that embodiment as any single embodiment or in combination with any other embodiments described herein.

### Definitions

Provided below are definitions of some of the terms. Additional definitions are set forth throughout the specification. Unless otherwise defined herein, all technical and scientific terms used herein have the same meaning as commonly understood by those of ordinary skill in the art.

“5-methylcytidine” (m⁵C) is a modified nucleoside derived from 5-methylcytosine. 5-Methylcytosine is a methylated form of the DNA base cytosine that may be involved in the regulation of gene transcription. See, e.g., WO 2013/052523.

“About” means, approximately the value stated. The term “about” “reflects the inherent uncertainty in any scientific measurement—i.e., repeated measurements of the same property will not yield exactly the same result due to the limitations of accuracy and precision associated with measurement and testing techniques.

“Analogs” include polynucleotide variants which differ by one or more modifications, e.g., substitutions, additions or deletions of nucleotide residues that still maintain one or more of the properties of the parent or starting polynucleotide.

The term “alter,” “altering,” “alteration of” or “altered” gene expression used herein refers to any action or process that is capable of modulating (interchangeably used with “altering,” “regulating,” “modifying,” “controlling” and“changing”) transcription and/or translation of a sequence of interest (e.g. a gene). Therefore, in one example, the alteration of gene expression includes any transcriptional regulation such as transcriptional activation (interchangeably used with “promotion,” “enhancement,” “increase” or “upregulation” of transcription) and transcriptional repression (interchangeably used with “reduction,” “decrease,” “inhibition” or “suppression” of transcription). In another example, the alteration of gene expression includes translational activation (interchangeably used with “promotion,” “enhancement,” “increase” or “upregulation” of transcription) and translational repression (interchangeably used with “reduction,” “decrease,” “inhibition” or “suppression” of transcription). In embodiments, the alteration of gene expression includes edition of nucleic acid sequence in genomic DNA. Thus, in embodiments the edition of nucleic acid sequence includes genome edition. In embodiments, the edition of nucleic acid sequence includes editing the sequence of non-genomic DNA or RNA (e.g. mRNA). In embodiments, the edition of nucleic acid sequence is done by mutating and/or deleting one or more nucleic acids from the sequence of interest (e.g. a genomic DNA sequence, non-genomic DNA sequence or RNA sequence), or inserting additional nucleic acid(s) into the sequence of interest.

The term “genome edition” or “editing genome” used herein refers to alteration of DNA sequence in a genome. The alternation of genome can be done by deletion of part of genomic DNA sequence, insertion of an additional DNA sequence into the genome and/or replacement of part of genome with a different DNA sequence. In embodiments, the edition of genome is permanent such that a daughter cell dived from the original cell that has the edited genome will have the same, altered (or modified) genome.

“Cas” refers to “CRISPR-associated” genes and proteins. CRISPR-Cas systems can be divided into two classes, Class 1 and Class 2, according to the configuration of their effector modules. CRISPR systems that may be used vary greatly. These systems will generally have the functional activities of a being able to form complex having a protein and a gRNA sequence where the complex recognizes a second nucleic acid. CRISPR systems can be a type I, a type II, or a type III system. Non-limiting examples of suitable CRISPR proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

“Cas9” molecule refers to a protein that can interact with a sgRNA molecule (e.g., sequence of a domain of a tracr) and, in concert with the sgRNA molecule, localize (“target” or “home”) to a site that comprises a target sequence and PAM sequence. Cas9 molecules of, derived from, or based on the Cas9 proteins of a variety of species can be used in the methods and compositions described in this specification. A “CRISPR associated protein 9,” “Cas9,” “Csn1” or “Cas9 protein” as referred to herein includes any of the recombinant or naturally-occurring forms of the Cas9 endonuclease or variants or homologs thereof that maintain Cas9 endonuclease enzyme activity (e.g. within at least 50%, 80%, 90%, 95%, 96%, 97%, 98%, 99% or 100% activity compared to Cas9). In some embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas9 protein. In embodiments, the Cas9 protein is substantially identical to the protein identified by the UniProt reference number Q99ZW2 or a variant or homolog having substantial identity thereto. Cas9 refers to the protein also known in the art as “nickase”. In embodiments, Cas9 is an RNA-guided DNA endonuclease enzyme that binds a CRISPR (clustered regularly interspaced short palindromic repeats) nucleic acid sequence. In embodiments, the CRISPR nucleic acid sequence is a prokaryotic nucleic acid sequence. In embodiments, the Cas9 nuclease from Streptococcus pyogenes is targeted to genomic DNA by a synthetic guide RNA consisting of a 20-nt guide sequence and a scaffold. The guide sequence base-pairs with the DNA target, directly upstream of a requisite 5′-NGG protospacer adjacent motif (PAM), and Cas9 mediates a double-stranded break (DSB) about 3-base pair upstream of the PAM. In embodiments, the CRISPR nuclease from Streptococcus aureus is targeted to genomic DNA by a synthetic guide RNA consisting of a 21-23-nt guide sequence and a scaffold. The guide sequence base-pairs with the DNA target, directly upstream of a requisite 5′-NNGRRT protospacer adjacent motif (PAM), and Cas9 mediates a double-stranded break (DSB) about 3-base pair upstream of the PAM.

The term “Cas9 variant” refers to proteins that have at least 80%, 85%, 90%, 95%, 96%, 97%, 98%, 99% or 100% amino acid sequence identity across the whole sequence or a functional portion of the sequence (e.g. a 50, 100, 150 or 200 continuous amino acid portion) compared to wild-type Cas9 protein and have one or more mutations that increase its binding specificity to PAM compared to wild-type Cas9 protein.

“Class 2” CRISPR systems use a large single-component Cas protein in conjunction with crRNAs to mediate interference. A class 2 CRISPR-Cas system can use Cas9. A class 2 CRISPR-Cas system can alternatively use Cpf1. See, e.g., Zetsche et al. (2015) Cell 163: 759-771. The term “Class II CRISPR endonuclease” refers to endonucleases that have similar endonuclease activity as Cas9 and participate in a Class II CRISPR system. An example Class II CRISPR system is the type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each).

“Cpf1” is an RNA-guided endonuclease of a class II CRISPR/Cas system found in Prevotella and Francisella bacteria. “CRISPR/Cpf1” is a DNA-editing technology analogous to the CRISPR/Cas9 system. Cpf1 is a smaller and simpler endonuclease than Cas9, overcoming some of the CRISPR/Cas9 system limitations. The term Cpf1 includes all orthologs, and variants that can be used in a CRISPR system. A “Cpf1” or “Cpf1 protein” as referred to herein includes any of the recombinant or naturally-occurring forms of the Cpf1 (Clustered Regularly Interspaced Short Palindromic Repeats from Prevotella and Francisella 1 or CRISPR/Cpf1) endonuclease or variants or homologs thereof that maintain Cpf1endonuclease enzyme activity (e.g. within at least 50%, 80%, 90%, 95%, 96%, 97%, 98%, 99% or 100% activity compared to Cpf1). In some embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cpf1 protein.

“CRISPR system” or “CRISPR-Cas system” comprises the transcripts and other elements involved in the activity of CRISPR-associated (Cas) genes, including sequences encoding a Cas gene or the Cas protein itself or both, a tracrRNA, a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system); RNAs (e.g., RNAs to guide Cas9, e.g. crRNA and tracrRNA or a single guide RNA (sgRNA) (chimeric RNA)); or other sequences and transcripts from a CRISPR locus. See, WO 2014/093622 A2 (The Broad Institute, Inc., Massachusetts Institute Of Technology). In general, a CRISPR system is characterized by elements that promote the formation of a CRISPR complex at the site of a target sequence (also referred to as a protospacer in the context of an endogenous CRISPR system). One of skill in the biotechnological art can identify direct repeats in silico by searching for repetitive motifs that fulfill any or all of the following criteria: (1) found in a 2 kb window of genomic sequence flanking the type II CRISPR locus; (2) span from 20 to 50 bp; and (3) interspaced by 20 to 50 bp. Two of these criteria can be used, e.g., 1 and 2, 2 and 3, or 1 and 3. Alternatively, all three criteria can be used. It might be preferred in a CRISPR complex that the tracr sequence has one or more hairpins and is 30 or more nucleotides in length, 40 or more nucleotides in length, or 50 or more nucleotides in length; the guide sequence is between 10 to 30 nucleotides in length, the CRISPR/Cas enzyme is a Type II Cas9 enzyme.

“CRISPR” refers to a set of Clustered Regularly Interspaced Short Palindromic repeats, or a system comprising such a set of repeats. Naturally occurring CRISPR systems confer resistance to foreign genetic elements, e.g., plasmids and phages. Naturally occurring CRISPR systems provide a form of acquired immunity. The CRISPR system is used in gene editing (silencing, enhancing or changing specific genes) in eukaryotes, e.g., mice, primates and humans, by, e.g., introducing into the eukaryotic cell one or more vectors encoding a specifically engineered guide RNA and one or more appropriate RNA-guided nucleases, e.g., Cas proteins. See, Wiedenheft et al. (2012) Nature 482: 331-8. In some prokaryotes, Cse (Cas subtype, Escherichia coli) proteins (e.g., CasA) form a functional complex, Cascade, which processes CRISPR RNA transcripts into spacer-repeat units that Cascade retains. Brouns et al. (2008) Science 321: 960-964. In other prokaryotes, Cas6 processes the CRISPR transcript. In Escherichia coli, CRISPR-based phage inactivation requires Cascade and Cas3, but not Cas1 or Cas2. In Pyrococcus furiosus and other prokaryotes, Cmr (Cas RAMP module) proteins form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA has been used in a system for gene editing. Pennisi (2013) Science 341: 833-836.

“Downstream” refers to the 5′ to 3′ direction in which RNA transcription takes place, so downstream is toward the 3′ end of an RNA molecule.

“E. coli RNA polymerase” is an RNA polymerase. The core enzyme consists of 5 subunits designated α, α, β′, β, and ω. The core enzyme is free of sigma factor and does not recognize any specific bacterial or phage DNA promoters, and so retains the ability to transcribe RNA from nonspecific initiation sequences. The holoenzyme is the core enzyme saturated with the addition of a sigma factor, which allows the enzyme to initiate RNA synthesis from specific bacterial and phage promoters.

“HDV ribozyme” is a self-cleaving RNA sequence derived from the hepatitis delta virus, having at least 90%, 95%, 98%, 99% or 100% sequence identity to SEQ ID NO. 5.

“In vitro transcription (IVT) cassette” includes a RNA polymerase promoter upstream from a transcription initiation nucleotide of an RNA sequence having a length of about 20-200 bases. The IVT cassette can include one or more of a linearization sequence, a ribozyme sequence, an RNA polymerase termination sequence, and one or more modified nucleotides.

“In vitro transcription” (IVT) is RNA transcription in vitro. Many kits for in vitro transcription are commercially available. New England Biolabs (Beverly, Mass., USA) sells the HiScribe™ T7 High Yield RNA Synthesis Kit.

“Initiation site” is the initiation site for RNA transcription. The initiation nucleotide can be selected to provide transcription with a selected RNA polymerase. For example, T7 polymerase promoter best transcribes when the initiating nucleotide is guanosine. Transcription from a modified T7 polymerase promoter can also begin with adenosine.

“Immune stimulating moiety” is a substance that potentiates and/or modulates the immune responses to an antigen to improve them.

“Linearization site” or “linearization sequence” can be recognition sites for restriction endonucleases (e.g. BspQI, DraI, SapI, BbsI, etc.).

“n+x product” (or “n+x mutation,” “n+x variant,” “n+x fragment”), when referring to an RNA transcript sample, describes the difference between the expected and the actual number of ribonucleotides in an RNA transcript. The “n” is the number of nucleotides in the transcript as expected from the DNA-coding region, while “x” is the additional number of non-template nucleotides in the actual, measured RNA transcript.

“n−x product” (or “n−x mutation,” “n−x variant,” “n−x fragment”), when referring to an RNA transcript sample, describes the difference between the expected and the actual number of ribonucleotides in an RNA transcript. The “n” is the number of nucleotides in the transcript as expected from the DNA-coding region, while “x” is the reduced number of non-template nucleotides in the actual, measured RNA transcript.

“Nucleic acid” refers to deoxyribonucleotides or ribonucleotides and polymers thereof in either single-, double- or multiple-stranded form, or complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like. The terms also encompass nucleic acids containing known nucleotide analogues or modified backbone residues or linkages, which are synthetic, naturally occurring, and non-naturally occurring, which have similar binding properties as the reference nucleic acid, and which are metabolized in a manner similar to the reference nucleotides. Examples of such analogues include, without limitation, phosphodiester derivatives including, e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press); and peptide nucleic acid backbones and linkages. Other analogue nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogues can be made; alternatively, mixtures of different nucleic acid analogues, and mixtures of naturally occurring nucleic acids and analogues may be made. In embodiments, the internucleotide linkages in DNA are phosphodiester, phosphodiester derivatives, or a combination of both. In some embodiments, modified nucleotides or nucleosides include chemical modifications such as a chemical substitution at a sugar position, a phosphate position, and/or a base position of the nucleic acid including, for example, incorporation of a modified nucleotide, incorporation of a capping moiety (e.g. 3′ capping), conjugation to a high molecular weight, non-immunogenic compound (e.g. polyethylene glycol (PEG)), conjugation to a lipophilic compound, substitutions in the phosphate backbone. Base modifications may include 5-position pyrimidine modifications, modifications at exocyclic amines, substitution of 4-thiouridine, substitution of 5-bromo- or 5-iodo-uracil, backbone modifications. Sugar modifications may include 2′-amine nucleotides (2′-NH₂), 2′-fluoro nucleotides (2′-F), and 2′-O-alkyl nucleotides (e.g., 2′-O-methyl (2′-OMe) nucleotides or 2′-O-(2-methoxyethyl) nucleotides). 2′-substituted nucleosides include 2′-fluoro, 2-deoxy, 2′-O-methyl, 2′-O-β-methoxyethyl, 2′-O-allylriboribonucleosides, 2′-amino, locked nucleic acid (LNA) monomers and the like. A wide range of nucleotide, nucleoside, base and phosphate modifications are known to those or ordinary skill in the art, e.g. as described in Eaton et al., Bioorganic & Medicinal Chemistry, Vol. 5, No. 6, pp 1087-1096, 1997

The term “nucleotide” typically refers to a compound containing a nucleoside or a nucleoside analogue and at least one phosphate group or a modified phosphate group linked to it by a covalent bond. Exemplary covalent bonds include, without limitation, an ester bond between the 3′, 2′ or 5′ hydroxyl group of a nucleoside and a phosphate group.

The term “nucleoside” refers to a compound containing a sugar part and a nucleobase, e.g. pyrimidine or purine base. Exemplary sugars include, without limitation, ribose, 2-deoxyribose, arabinose and the like. Exemplary nucleobases include, without limitation, thymine, uracil, cytosine, adenine, guanine.

“Partially ssDNA oligo template” includes dsDNA portion and single stranded portion. The double stranded portion can encode all of a portion of the sgRNA. The single stranded portion can be complimentary to the sequence encoding all or a portion of an RNA polymerase promoter enhancing sequence and/or an RNA polymerase promoter.

“Plasmid based template” consists of IVT cassette inserted into appropriate vector for amplification of plasmid DNA

“Polynucleotide variant” refers to molecules that differ in their nucleotide sequence from a native or reference sequence, which can possess substitutions, deletions, or insertions at certain positions within the encoded amino acid sequence, as shown in WO 2015/006747 A2.

“Polynucleotide” includes any compound or substance that comprises a polymer of nucleotides, as shown in WO 2015/006747 A2.

“Pseudouridine” (P) is an isomer of the nucleoside uridine in which the uracil is attached via a carbon-carbon instead of a nitrogen-carbon glycosidic bond. See, WO WO2013/052523 A1.

“Purity” or “purified” refers to the level of contaminates (undesired product, e.g., residual DNA, n+x product, n−x product) in the final product/composition prepared according to the methods or processes described herein as being less than 5% by weight, less than 4% by weight, less than 3% by weight, less than 2% by weight, less than 1% by weight, less than 0.5% by weight, less than 0.1% by weight, less than 0.05% by weight or less than 0.01% by weight. Purity can be measured by any methods appropriately known in the art. In some embodiments, the purity is determined by chromatograms UV260 nm.

“Ribozyme” and “ribozyme sequence” is a self-cleaving RNA sequences that is inserted after the end of the RNA sequence. Upon transcription, the ribozyme sequence cleaves off, leaving a precise end to the RNA. This method is particularly useful if no unique restriction sites are available for linearization. One example of a ribozyme is a hepatitis delta (HDV) ribozyme of SEQ ID NO: 5.

“RNA polymerase promoter” can be, but is not limited to, a T7 promoter, a T3 promoter, a SP6 promoter, a promoter recognized by cyanophage Syn5 RNA polymerase, or a promoter recognized by E. coli RNA polymerase, as described in WO 2015/024017 A2. Those of skill in the biotechnological arts will know the nucleotide sequences of other RNA polymerase promoters

The terms “guide RNA,” “guide RNA molecule,” “gRNA molecule” or “gRNA” are used interchangeably, and refer to a set of nucleic acid molecules that promote the specific directing of a RNA-guided nuclease or other effector molecule (typically in complex with the gRNA molecule) to a target sequence. In some embodiments, said directing is accomplished through hybridization of a portion of the gRNA to DNA (e.g., through the gRNA targeting domain), and by binding of a portion of the gRNA molecule to the RNA-guided nuclease or other effector molecule (e.g., through at least the gRNA tracr). In embodiments, a gRNA molecule consists of a single contiguous polynucleotide molecule, referred to herein as a “single guide RNA” or “sgRNA” and the like. In embodiments, sgRNA includes the crRNA sequence and optionally the tracrRNA sequence. In embodiments, sgRNA includes the crRNA sequence. In embodiments, sgRNA includes the crRNA sequence and the tracrRNA sequence. The term “targeting domain” as the term is used in connection with a gRNA, is the portion of the gRNA molecule that recognizes, e.g., is complementary to, a target sequence, e.g., a target sequence within the nucleic acid of a cell, e.g., within a gene. The term “crRNA” as the term is used in connection with a gRNA molecule, is a portion of the gRNA molecule that comprises a targeting domain and a region that interacts with a tracr to form a flagpole region. The term “flagpole” as used herein in connection with a gRNA molecule, refers to the portion of the gRNA where the crRNA and the tracr bind to, or hybridize to, one another.

In some embodiments, the degree of complementarity between a targeting domain and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). The term “complementary” as used in connection with nucleic acid, refers to the pairing of bases, A with T or U, and G with C. The term complementary refers to nucleic acid molecules that are completely complementary, that is, form A to T or U pairs and G to C pairs across the entire reference sequence, as well as molecules that are at least 80%, 85%, 90%, 95%, 99% complementary.

In embodiments, the length of sgRNA sequence is 50-150 bases (e.g., 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 bases).

In embodiments, the length of sgRNA sequence is 50-120 bases (e.g., 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 bases).

In embodiments, the length of sgRNA sequence is 60-120 bases (e.g., 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 bases).

In one embodiment, the sgRNA sequence comprises a tracrRNA sequence having at least 90%, 95%, 98%, 99% or 100% sequence identity to SEQ ID NO: 5. In another embodiment, the sgRNA sequence comprises a sequence having at least 90%, 95%, 98%, 99% or 100% sequence identity to SEQ ID NO: 6. In another embodiment, the sgRNA sequence comprises a sequence having at least 90%, 95%, 98%, 99% or 100% sequence identity to SEQ ID NO: 7. In one embodiment, the sgRNA sequence comprises a sequence having at least 90%, 95%, 98%, 99% or 100% sequence identity to SEQ ID NO: 33. In one embodiment, the sgRNA sequence comprises a sequence having at least 90%, 95%, 98%, 99% or 100% sequence identity to SEQ ID NO: 34. In one embodiment, the sgRNA sequence comprises a sequence having at least 90%, 95%, 98%, 99% or 100% sequence identity to SEQ ID NO: 35. In one embodiment, the sgRNA sequence comprises a sequence having at least 90%, 95%, 98%, 99% or 100% sequence identity to SEQ ID NO: 36. In one embodiment, the sgRNA sequence comprises a sequence having at least 90%, 95%, 98%, 99% or 100% sequence identity to SEQ ID NO: 37. In one embodiment, the sgRNA sequence comprises a sequence having at least 90%, 95%, 98%, 99% or 100% sequence identity to SEQ ID NO: 38. In one embodiment, the sgRNA sequence comprises a sequence having at least 90%, 95%, 98%, 99% or 100% sequence identity to SEQ ID NO: 39. In one embodiment, the sgRNA sequence comprises a sequence having at least 90%, 95%, 98%, 99% or 100% sequence identity to SEQ ID NO: 40. In one embodiment, the sgRNA sequence comprises a sequence having at least 90%, 95%, 98%, 99% or 100% sequence identity to SEQ ID NO: 41. In one embodiment, the sgRNA sequence comprises a sequence having at least 90%, 95%, 98%, 99% or 100% sequence identity to SEQ ID NO: 42. In one embodiment, the sgRNA sequence comprises a sequence having at least 90%, 95%, 98%, 99% or 100% sequence identity to SEQ ID NO: 43. In one embodiment, the sgRNA sequence comprises a sequence having at least 90%, 95%, 98%, 99% or 100% sequence identity to SEQ ID NO: 44. In one embodiment, the sgRNA sequence comprises a sequence having at least 90%, 95%, 98%, 99% or 100% sequence identity to SEQ ID NO: 45. In one embodiment, the sgRNA sequence comprises a sequence having at least 90%, 95%, 98%, 99% or 100% sequence identity to SEQ ID NO: 46. In one embodiment, the sgRNA sequence comprises a sequence having at least 90%, 95%, 98%, 99% or 100% sequence identity to SEQ ID NO: 47. In one embodiment, the sgRNA sequence comprises a sequence having at least 90%, 95%, 98%, 99% or 100% sequence identity to SEQ ID NO: 48. In one embodiment, the sgRNA sequence comprises a sequence having at least 90%, 95%, 98%, 99% or 100% sequence identity to SEQ ID NO: 49. In one embodiment, the sgRNA sequence comprises a sequence having at least 90%, 95%, 98%, 99% or 100% sequence identity to SEQ ID NO: 50. In one embodiment, the sgRNA sequence comprises a sequence having at least 90%, 95%, 98%, 99% or 100% sequence identity to SEQ ID NO: 51.

In some embodiments, the sgRNA may comprise, from 5′ to 3′, disposed 3′ to the targeting domain:

a)
(SEQ ID NO: 52)
GUUUUAGAGCUAGAAAUAGCAAGUUAAAAUAAGGCUAGUCCGUUAUCAAC
UUGAAAAAGUGGCACCGAGUCGGUGC;
b)
(SEQ ID NO: 53)
GUUUAAGAGCUAGAAAUAGCAAGUUUAAAUAAGGCUAGUCCGUUAUCAAC
UUGAAAAAGUGGCACCGAGUCGGUGC;
c)
(SEQ ID NO: 54)
GUUUUAGAGCUAUGCUGGAAACAGCAUAGCAAGUUAAAAUAAGGCUAGUC
CGUUAUCAACUUGAAAAAGUGGCACCGAGUCGGUGC;
d)
(SEQ ID NO: 55)
GUUUAAGAGCUAUGCUGGAAACAGCAUAGCAAGUUUAAAUAAGGCUAGUC
CGUUAUCAACUUGAAAAAGUGGCACCGAGUCGGUGC;

e) any of a) to d), above, further comprising, at the 3′ end, at least 1, 2, 3, 4, 5, 6 or 7 uracil (U) nucleotides, e.g., 1, 2, 3, 4, 5, 6, or 7 uracil (U) nucleotides;
f) any of a) to d), above, further comprising, at the 3′ end, at least 1, 2, 3, 4, 5, 6 or 7 adenine (A) nucleotides, e.g., 1, 2, 3, 4, 5, 6, or 7 adenine (A) nucleotides; or
g) any of a) to 0, above, further comprising, at the 5′ end (e.g., at the 5′ terminus, e.g., 5′ to the targeting domain), at least 1, 2, 3, 4, 5, 6 or 7 adenine (A) nucleotides, e.g., 1, 2, 3, 4, 5, 6, or 7 adenine (A) nucleotides. In embodiments, any of a) to g) above is disposed directly 3′ to the targeting domain.

In an embodiment, a sgRNA comprises, e.g., consists of, from 5′ to 3′: [targeting domain]—

(SEQ ID NO: 56)
GUUUUAGAGCUAGAAAUAGCAAGUUAAAAUAAGGCUAGUCCGUUAUCAAC
UUGAAAAAGUGGCACCGAGUCGGUGCUUUU.

In an embodiment, a sgRNA described herein comprises, e.g., consists of, from 5′ to 3′: [targeting domain]—

(SEQ ID NO: 57)
GUUUAAGAGCUAUGCUGGAAACAGCAUAGCAAGUUUAAAUAAGGCUAGUC
CGUUAUCAACUUGAAAAAGUGGCACCGAGUCGGUGCUUUU.

In embodiments, a sgRNA described herein comprises, e.g., consists of, a ribonucleic acid having the sequence:

(SEQ ID NO: 7)
NNNNNNNNNNNNNNNNNNNGUUUUAGAGCUAGAAAUAGCAAGUUAAAAUA
AGGCUAGUCCGUUAUCAACUUGAAAAAGUGGCACCGAGUCGGUGC,

where the n's refer to the residues of the targeting domain.

In an embodiment, a sgRNA described herein comprises, e.g., consists of:

(SEQ ID NO: 58)
NNNNNNNNNNNNNNNNNNNNGUUUUAGAGCUAGAAAUAGCAAGUUAAAAU
AAGGCUAGUCCGUUAUCAACUUGAAAAAGUGGCACCGAGUCGGUGCUUU
U,

where m indicates a base with 2′O-Methyl modification, * indicates a phosphorothioate bond, and N's indicate the residues of the targeting domain, e.g., as described herein, (optionally with an inverted abasic residue at the 5′ and/or 3′ terminus).

Other exemplary sgRNA molecules and their sequences can be found in WO2017115268 and WO2018142364, the contents of which are incorporated herein.

In some embodiments, a crRNA comprises, from 5′ to 3′, preferably disposed directly 3′ to the targeting domain:

(SEQ ID NO: 59)
a) GUUUUAGAGCUA;
(SEQ ID NO: 60)
b) GUUUAAGAGCUA;
(SEQ ID NO: 61)
c) GUUUUAGAGCUAUGCUG;
(SEQ ID NO: 62)
d) GUUUAAGAGCUAUGCUG;
(SEQ ID NO: 63)
e) GUUUUAGAGCUAUGCUGUUUUG;
(SEQ ID NO: 64)
f) GUUUAAGAGCUAUGCUGUUUUG;
or
(SEQ ID NO: 65)
g) GUUUUAGAGCUAUGCU.

In some embodiments, a tracr comprises, from 5′ to 3′:

a)
(SEQ ID NO: 66)
UAGCAAGUUAAAAUAAGGCUAGUCCGUUAUCAACUUGAAAAAGUGGCACC
GAGUCGGUGC;
b)
(SEQ ID NO: 67)
UAGCAAGUUUAAAUAAGGCUAGUCCGUUAUCAACUUGAAAAAGUGGCACC
GAGUCGGUGC;
c)
(SEQ ID NO: 68)
CAGCAUAGCAAGUUAAAAUAAGGCUAGUCCGUUAUCAACUUGAAAAAGUG
GCACCGAGUCGGUGC;
d)
(SEQ ID NO: 69)
CAGCAUAGCAAGUUUAAAUAAGGCUAGUCCGUUAUCAACUUGAAAAAGUG
GCACCGAGUCGGUGC;
e)
(SEQ ID NO: 70)
AACAGCAUAGCAAGUUAAAAUAAGGCUAGUCCGUUAUCAACUUGAAAAAG
UGGCACCGAGUCGGUGCUUUUUUU;
f)
(SEQ ID NO: 71)
AACAGCAUAGCAAGUUUAAAUAAGGCUAGUCCGUUAUCAACUUGAAAAAG
UGGCACCGAGUCGGUGCUUUUUUU;
g)
(SEQ ID NO: 72)
AACAGCAUAGCAAGUUAAAAUAAGGCUAGUCCGUUAUCAACUUGAAAAAG
UGGCACCGAGUCGGUGC
h)
(SEQ ID NO: 73)
GUUUAAGAGCUAUGCUGGAAACAGCAUAGCAAGUUUAAAUAAGGCUAGUC
CGUUAUCAACUUGAAAAAGUGGCACCGAGUCGGUGCUUUU;
i)
(SEQ ID NO: 74)
AGCAUAGCAAGUUAAAAUAAGGCUAGUCCGUUAUCAACUUGAAAAAGUGG
CACCGAGUCGGUGCUUU;
j)
(SEQ ID NO: 75)
GUUGGAACCAUUCAAAACAGCAUAGCAAGUUAAAAUAAGGCUAGUCCGUU
AUCAACUUGAAAAAGUGGCACCGAGUCGGUGCUUU;

k) any of a) to j), above, further comprising, at the 3′ end, at least 1, 2, 3, 4, 5, 6 or 7 uracil (U) nucleotides, e.g., 1, 2, 3, 4, 5, 6, or 7 uracil (U) nucleotides;
l) any of a) to j), above, further comprising, at the 3′ end, at least 1, 2, 3, 4, 5, 6 or 7 adenine (A) nucleotides, e.g., 1, 2, 3, 4, 5, 6, or 7 adenine (A) nucleotides; or
m) any of a) to l), above, further comprising, at the 5′ end (e.g., at the 5′ terminus), at least 1, 2, 3, 4, 5, 6 or 7 adenine (A) nucleotides, e.g., 1, 2, 3, 4, 5, 6, or 7 adenine (A) nucleotides.

In an embodiment, the sequence of k), above comprises the 3′ sequence UUUUUU, e.g., if a U6 promoter is used for transcription. In an embodiment, the sequence of k), above, comprises the 3 sequence UUUU, e.g., if an HI promoter is used for transcription. In an embodiment, sequence of k), above, comprises variable numbers of 3′ U's depending, e.g., on the termination signal of the pol-III promoter used. In an embodiment, the sequence of k), above, comprises variable 3′ sequence derived from the DNA template if a T7 promoter is used. In an embodiment, the sequence of k), above, comprises variable 3 sequence derived from the DNA template, e.g., if in vitro transcription is used to generate the RNA molecule. In an embodiment, the sequence of k), above, comprises variable 3′ sequence derived from the DNA template, e.g., if a pol-II promoter is used to drive transcription.

Other exemplary gRNA (crRNA and/or tracrRNA), sgRNA molecules and their sequences can be found in WO2017115268 and WO2018142364, the contents of which are incorporated herein.

“Sequence identity”. Percent identity of two amino acid sequences, or of two nucleic acid sequences is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux et al., Nucleic Acids Research 12(1): 387, 1984), BLASTP, BLASTN, and FASTA (Altschul et al. J. Mol. Biol. 215: 403-410, 1990). The BLAST X program is publicly available from NCBI and other sources (BLAST Manual, Altschul et al. NCBI NLM NIH Bethesda, Md. 20894; Altschul et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

“SP6 promoter” is a polynucleotide sequence for a SP6 RNA polymerase to begin transcription, preferably with at least 90%, 95%, 98%, 99% or 100% sequence identity to SEQ ID NO: 12. Transcription initiates on the first nucleotide following the promoter sequence (typically guanosine).

A “surface coated” substrate is a substrate that is coated with a reagent that binds to a nonradiolabeled tagged probe. The substrate of the surface coated substrate can be magnetic beads. For example, Oligo dT magnetic beads are commercially available.

“Syn5 promoter” is a polynucleotide sequence for the marine cyanophage Syn5 RNA polymerase to begin transcription, preferably with at least 90%, 95%, 98%, 99% or 100% sequence identity to SEQ ID NO: 13. See, US 2016/0369248 A1 (President and Fellows of Harvard College). See also, Zhu et al. (1 Feb. 2013) J. Biol. Chem. 288(5): 3545-3552.

“Solid-phase chemical synthesis” is method in which molecules are bound, attached or adhered on a solid support, e.g., a bead, and synthesized step-by-step in a reactant solution; compared with normal synthesis in a liquid state, it is easier to remove excess reactant or byproduct from the product. In this method, building blocks are protected at all reactive functional groups. The two functional groups that are able to participate in the desired reaction between building blocks in the solution and on the bead can be controlled by the order of deprotection. Solid-phase chemical synthesis of relatively short fragments of nucleic acids with defined chemical structure (sequence) is useful in current laboratory practice because it provides a rapid and inexpensive access to custom-made oligonucleotides of the desired sequence. See, Sanghvi (2011) Curr. Protoc. Nucleic Acid Chem. 46 (16): 4.1.1-4.1.22. Some companies providing commercial include Axolabs (Kulmbach, Germany), Integrated DNA Technologies (IDT) (Coralville, Iowa, USA) and Biospring (Frankfurt, Germany).

As used herein, the term “substantially free” as used herein means that the undesired component (e.g., residual DNA, n+x product or n−x product, or immune stimulating moieties) is present in the composition described herein in an amount less than 5% by weight, less than 4% by weight, less than 3% by weight, less than 2% by weight, less than 1% by weight, less than 0.5% by weight, less than 0.1% by weight, less than 0.05% by weight, or less than 0.01% by weight.

“T3 RNA polymerase promoter” is a polynucleotide sequence for a T7 RNA polymerase to begin transcription, preferably with at least 90%, 95%, 98%, 99% or 100% sequence identity to SEQ ID NO. 11. Transcription initiates on the first nucleotide following the promoter sequence (usually guanosine).

“T7 RNA polymerase promoter upstream enhancer sequence” is an enhancer polynucleotide sequence upstream from the T7 RNA polymerase promoter, which helps to increase the yield of RNA in an IVT reaction, preferably with at least 90%, 95%, 98%, 99% or 100% sequence identity to SEQ ID NO: 6.

“T7 RNA polymerase promoter” is a polynucleotide sequence for a T7 RNA polymerase to begin transcription, preferably with at least 90%, 95%, 98%, 99% or 100% sequence identity to SEQ ID NO. 1. Transcription initiates on the first nucleotide following the promoter sequence (typically guanosine).

“Target DNA” is the DNA of interest that comprises a nucleotide sequence (the target sequence) to which the crRNA binds by Watson-Crick base pairing.

“Target sequence” refers to a sequence to which a guide sequence (e.g., a gRNA targeting domain) is designed to have complementarity, where hybridization between a target sequence and a guide sequence promotes the formation of a CRISPR complex. A target sequence can comprise any polynucleotide, such as DNA or RNA polynucleotides. A target sequence can be located in the nucleus or cytoplasm of a cell.

“tracrRNA” (trans-activating CRISPR) is the portion of sgRNA that binds to Cas9. tracrRNA is called an “activator-RNA” in in WO 2013/176772 A1. The portion of sgRNA that binds to Cas9 is constant.

“Transcription initiation nucleotide” is the first nucleotide from which transcription begins. A transcription initiation nucleotide could be A, T, C or G, depending on promoter and RNA polymerase chosen for specific transcript.

“Transcript” used herein refers to a polynucleotide of ribonucleotides having a length of about 20-200 bases (e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 bases), which is transcribed from a DNA template described herein through the process/method (e.g., IVT) described herein. In an embodiment, “transcript” is also referred as IVT-made transcript or IVT-made polynucleotide or IVT-made RNA. In an embodiment, transcript described herein is an IVT-made gRNA (crRNA or tracrRNA). In an embodiment, transcript described herein is an IVT-made sgRNA.

“Upstream” refers to the 5′ to 3′ direction in which RNA transcription takes place, so downstream is toward the 5′ end of an RNA molecule.

### IVT Cassettes, Compositions and Methods

The disclosure is directed to polynucleotides and methods of generating, characterizing and analyzing polynucleotides (e.g., RNAs having a length of about 20-200 bases, for example, guide RNAs (gRNAs) and single guide RNAs (sgRNAs)). The polynucleotides, e.g., RNAs having a length of about 20-200 bases, for example, gRNA and/or sgRNA, can be used to modulate transcription, e.g., in clinical or research settings. The disclosure provides an improvement in manufacturing RNAs having a length of about 20-200 bases and quality. By practicing the methods described herein, the variety of contaminants in a composition of full-length product (FLP) RNA transcript produced by in vitro transcription (IVT) is less than the corresponding composition of transcript produced by solid-phase chemical synthesis.

In solid-phase chemical synthesis of long ˜100 mer RNA oligonucleotides, as shown in FIG. 25 of FLUOROUS CHEMISTRY, EDITORS: HORVÁTH, ISTVÁN T. (ED.), the variety of oligonucleotide impurities than can occur is much greater than from IVT synthesis of RNA. Impurities can originate from incomplete addition of nucleotides, forming so-called “n−x truncated” fragments (also referred to herein as “n−x variants”), whose synthesis has been prematurely terminated. Also, an inefficient capping of sequences that have failed to incorporate a nucleotide results in the formation of oligonucleotides with internal deletions, which are also n−x fragments. Moreover, inefficient detritylation can result in other n−x fragments. Additional side-reactions in solid-phase chemical synthesis can occur because of the repeated exposure of the growing oligonucleotide chain to chemicals. Premature detritylation during coupling results in n+x fragments (also referred to herein as “n+x variants”) that have duplicated nucleotides in the sequence. Depurination during the detritylation step results in the formation of oligonucleotide products with abasic sites, which are later cleaved by ammonia during the deprotection stage. Minimizing undesired side reactions during chemical oligonucleotide synthesis requires protecting groups attached to the nucleosides during the chain elongation. Upon the completion of the oligonucleotide chain assembly, the protecting groups are removed to yield the desired oligonucleotides. Thus, other side products such as oligomers carrying residual protecting groups arising from incomplete deprotection, acrylamide adducts, bicyclic products, etc. can occur. These side products have previously been problematic to remove from the composition of the desired RNA transcript. In general, the longer the RNA chain, the more challenging the solid-phase synthesis is getting. In fact, even in cases of high coupling efficiencies (>99%) the percentage of side-products, generated with every nucleotide addition, accumulates drastically when oligomer the oligomer length is growing beyond >50 mer. The general relationship between full-length product (FLP) yield, oligonucleotide length, and various coupling efficiencies is that small decreases in coupling efficiency (51%) result in large decreases in full-length product (FLP) yield, most notably for long oligonucleotides. Because these various side-products are difficult (if not impossible) to remove, there is a risk that corresponding RNA compositions trigger unwanted off-targeting effects caused by the impurities contained in RNA sequence in compositions generated by chemical synthesis. The biggest risks are mutations in the crRNA region.

Also, because the chemical synthesis of long oligonucleotides has a very low yield, the overall cost of chemical synthesis will be higher than that of IVT.

In addition, it had been described in the art that IVT is not recommended for generating gRNA, allegedly due to three main reasons: low purity, variable efficiency and high cost (see, e.g., www.synthego.com/resources/3-Reasons-to-Stop-Using-IVT).

The compositions and methods described herein, therefore, provide unexpected solutions to some of the problems of chemical synthesis and other problems known in the art.

The present disclosure overcomes some of the deficiencies of chemical synthesis by allowing production of a composition of polynucleotides (e.g., RNAs having a length of about 20-200 bases, such as gRNA, sgRNA) having less than 6%, 5%, 4%, 3%, 2%, 1% or no detectable n−x fragments, preferably less than 4%, 3%, 2%, 1% or no detectable n−x fragments. n−x fragments can be detected by any methods known in the art, for example, by LC-MS or Next generation sequencing (NGS), ion exchange chromatography, reversed phase chromatography, or electrophoresis.

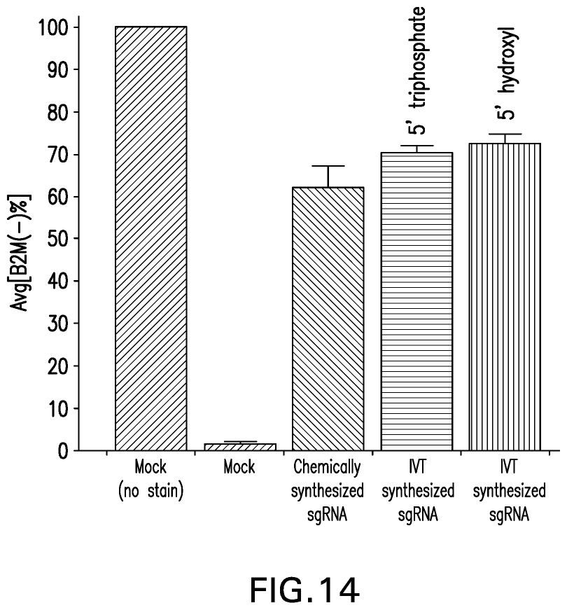

In embodiments, the percentage of desired product (e.g., RNA molecules having a length of about 20-200 bases, for example, gRNAs, sgRNAs, RNA aptamers, RNAi molecules, etc.) among IVT product is at least 50%, 55%, 60%, 65%, 70%, 75%, 80%, 85%, 90%, 95%, 100%, 150%, 200% or higher than the percentage of desired product among the chemically synthesized product. In other words, in embodiments, the purity of IVT product described herein is at least 50%, 55%, 60%, 65%, 70%, 75%, 80%, 85%, 90%, 95%, 100%, 150%, 200% or higher than the purity of the chemically synthesized product (see, e.g., FIG. 14).

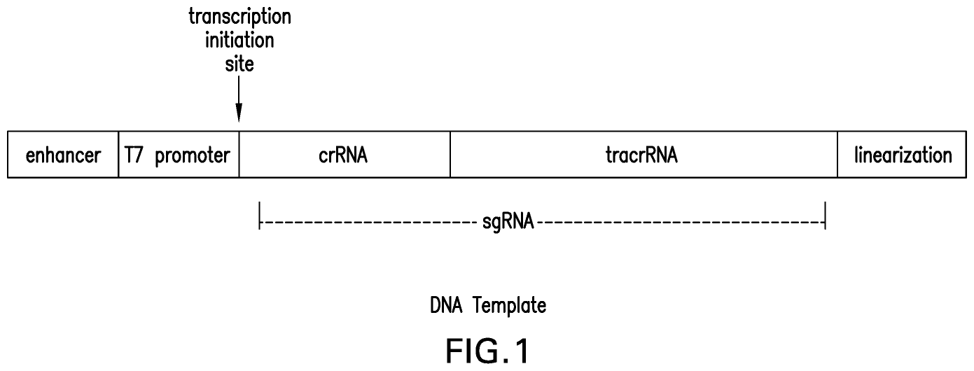

In one aspect, the disclosure features a DNA template for making a ribonucleic acid (RNA) transcript having a length of about 20-200 bases by in vitro transcription (IVT). The DNA template comprises an IVT cassette, which comprises a first DNA sequence including an RNA transcription initiation site, a polymerase promoter upstream from the RNA transcription initiation site, a second DNA sequence encoding the RNA transcript having a length of about 20-200 bases disposed downstream of the RNA transcription initiation site, and a linearization site downstream from the transcription initiation site (e.g., the downstream from the second DNA sequence). In some embodiments, the RNA transcript having a length of about 20-200 bases comprises a gRNA. In some embodiments, the gRNA is about 20-150 bases in length. In some embodiments, the RNA transcript having a length of about 20-200 bases comprises a sgRNA. In some embodiments, the sgRNA is about 50-150 bases in length. In some embodiments, the sgRNA sequence encodes a fusion transcript, which comprises crRNA and optionally tracrRNA. In some embodiments, the sgRNA sequence starts with a transcription initiation nucleotide. FIG. 1 shows a drawing of an exemplary IVT cassette, comprising a DNA sequence encoding the two sgRNA elements, crRNA and optionally tracrRNA. In some embodiments, the linearization site is immediately downstream of the second DNA sequence encoding the RNA transcript having a length of about 20-200 bases (e.g., the sgRNA sequence), near or at the end of the second DNA sequence, to keep the resulting RNA transcript at a desired length.

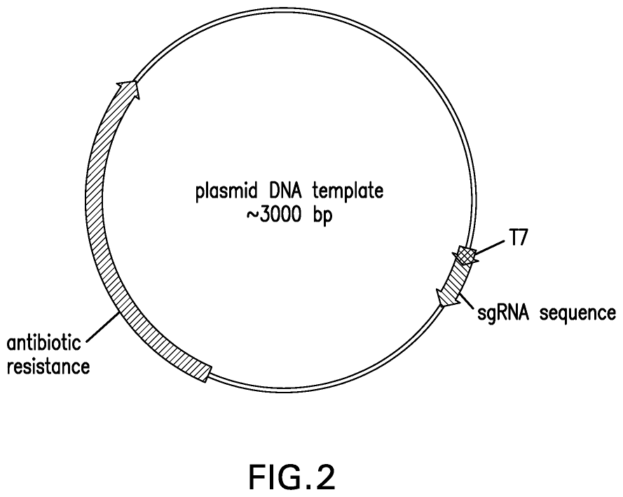

In one embodiment, the DNA template is part of a DNA plasmid, which comprises the IVT cassette and an appropriate vector for amplification of DNA, e.g., so that the plasmid can be amplified by growing in bacteria, e.g., Escherichia coli. See, FIG. 2.

In one embodiment, the promoter is an RNA polymerase promoter, e.g., selected from a T7 promoter, a T3 promoter, a SP6 promoter, a Syn5 promoter, a phi 2.5 overlapping promoter, an AC15/C26 mutA promoter, an A6/B1 mutA promoter, and a phi 9 (A-15C) promoter. In one embodiment, the promoter is a T7 promoter, e.g., having a sequence with at least 90%, 95%, 98%, 99% or 100% sequence identity to SEQ ID NO: 1. In another embodiment, the promoter is a T3 promoter, e.g., having a sequence with at least 90%, 95%, 98%, 99% or 100% sequence identity to SEQ ID NO: 2. In another embodiment, the promoter is a SP6 promoter, e.g., having a sequence with at least 90%, 95%, 98%, 99% or 100% sequence identity to SEQ ID NO: 3. In yet another embodiment, the promoter is a Syn5 promoter, e.g., having a sequence with at least 90%, 95%, 98%, 99% or 100% sequence identity to SEQ ID NO: 4. In yet another embodiment, the promoter is a phi 2.5 overlapping promoter, e.g., having a sequence with at least 90%, 95%, 98%, 99% or 100% sequence identity to SEQ ID NO: 27. In yet another embodiment, the promoter is an AC15/C26 mutA promoter, e.g., having a sequence with at least 90%, 95%, 98%, 99% or 100% sequence identity to SEQ ID NO: 28. In yet another embodiment, the promoter is an A6/B1 mutA promoter, e.g., having a sequence with at least 90%, 95%, 98%, 99% or 100% sequence identity to SEQ ID NO: 29. In yet another embodiment, the promoter is a phi 9 (A-15C) promoter, e.g., having a sequence with at least 90%, 95%, 98%, 99% or 100% sequence identity to SEQ ID NO: 30. The nucleotide sequences of other RNA polymerase promoters (e.g., promoters for E. coli RNA polymerase) are known in the art.

In one embodiment, the RNA transcription initiation site has adenosine as the initiating nucleotide. In one embodiment, where the RNA polymerase promoter is a T7 promoter, the initiation site has adenosine as the initiating nucleotide. In another embodiment, the RNA transcription initiation site has guanosine as the initiating nucleotide. In one embodiment, where the RNA polymerase promoter is a T7 promoter, the initiation site has guanosine as the initiating nucleotide.

In one embodiment, the sgRNA sequence comprises a tracrRNA sequence having at least 90%, 95%, 98%, 99% or 100% sequence identity to SEQ ID NO: 5. In another embodiment, the sgRNA sequence comprises a sequence having at least 90%, 95%, 98%, 99% or 100% sequence identity to SEQ ID NO: 6. In another embodiment, the sgRNA sequence comprises a sequence having at least 90%, 95%, 98%, 99% or 100% sequence identity to SEQ ID NO: 7. In one embodiment, the sgRNA sequence comprises a sequence having at least 90%, 95%, 98%, 99% or 100% sequence identity to SEQ ID NO: 33. In one embodiment, the sgRNA sequence comprises a sequence having at least 90%, 95%, 98%, 99% or 100% sequence identity to SEQ ID NO: 34. In one embodiment, the sgRNA sequence comprises a sequence having at least 90%, 95%, 98%, 99% or 100% sequence identity to SEQ ID NO: 35. In one embodiment, the sgRNA sequence comprises a sequence having at least 90%, 95%, 98%, 99% or 100% sequence identity to SEQ ID NO: 36. In one embodiment, the sgRNA sequence comprises a sequence having at least 90%, 95%, 98%, 99% or 100% sequence identity to SEQ ID NO: 37. In one embodiment, the sgRNA sequence comprises a sequence having at least 90%, 95%, 98%, 99% or 100% sequence identity to SEQ ID NO: 38. In one embodiment, the sgRNA sequence comprises a sequence having at least 90%, 95%, 98%, 99% or 100% sequence identity to SEQ ID NO: 39. In one embodiment, the sgRNA sequence comprises a sequence having at least 90%, 95%, 98%, 99% or 100% sequence identity to SEQ ID NO: 40. In one embodiment, the sgRNA sequence comprises a sequence having at least 90%, 95%, 98%, 99% or 100% sequence identity to SEQ ID NO: 41. In one embodiment, the sgRNA sequence comprises a sequence having at least 90%, 95%, 98%, 99% or 100% sequence identity to SEQ ID NO: 42. In one embodiment, the sgRNA sequence comprises a sequence having at least 90%, 95%, 98%, 99% or 100% sequence identity to SEQ ID NO: 43. In one embodiment, the sgRNA sequence comprises a sequence having at least 90%, 95%, 98%, 99% or 100% sequence identity to SEQ ID NO: 44. In one embodiment, the sgRNA sequence comprises a sequence having at least 90%, 95%, 98%, 99% or 100% sequence identity to SEQ ID NO: 45. In one embodiment, the sgRNA sequence comprises a sequence having at least 90%, 95%, 98%, 99% or 100% sequence identity to SEQ ID NO: 46. In one embodiment, the sgRNA sequence comprises a sequence having at least 90%, 95%, 98%, 99% or 100% sequence identity to SEQ ID NO: 47. In one embodiment, the sgRNA sequence comprises a sequence having at least 90%, 95%, 98%, 99% or 100% sequence identity to SEQ ID NO: 48. In one embodiment, the sgRNA sequence comprises a sequence having at least 90%, 95%, 98%, 99% or 100% sequence identity to SEQ ID NO: 49. In one embodiment, the sgRNA sequence comprises a sequence having at least 90%, 95%, 98%, 99% or 100% sequence identity to SEQ ID NO: 51. In one embodiment, the sgRNA sequence comprises a sequence having at least 90%, 95%, 98%, 99% or 100% sequence identity to SEQ ID NO: 51.

In some embodiments, the sgRNA may comprise, from 5′ to 3′, disposed 3′ to the targeting domain:

a)
(SEQ ID NO: 52)
GUUUUAGAGCUAGAAAUAGCAAGUUAAAAUAAGGCUAGUCCGUUAUCAAC
UUGAAAAAGUGGCACCGAGUCGGUGC;
b)
(SEQ ID NO: 53)
GUUUAAGAGCUAGAAAUAGCAAGUUUAAAUAAGGCUAGUCCGUUAUCAAC
UUGAAAAAGUGGCACCGAGUCGGUGC;
c)
(SEQ ID NO: 54)
GUUUUAGAGCUAUGCUGGAAACAGCAUAGCAAGUUAAAAUAAGGCUAGUC
CGUUAUCAACUUGAAAAAGUGGCACCGAGUCGGUGC;
d)
(SEQ ID NO: 55)
GUUUAAGAGCUAUGCUGGAAACAGCAUAGCAAGUUUAAAUAAGGCUAGUC
CGUUAUCAACUUGAAAAAGUGGCACCGAGUCGGUGC;

e) any of a) to d), above, further comprising, at the 3′ end, at least 1, 2, 3, 4, 5, 6 or 7 uracil (U) nucleotides, e.g., 1, 2, 3, 4, 5, 6, or 7 uracil (U) nucleotides;
f) any of a) to d), above, further comprising, at the 3′ end, at least 1, 2, 3, 4, 5, 6 or 7 adenine (A) nucleotides, e.g., 1, 2, 3, 4, 5, 6, or 7 adenine (A) nucleotides; or
g) any of a) to f), above, further comprising, at the 5′ end (e.g., at the 5′ terminus, e.g., 5′ to the targeting domain), at least 1, 2, 3, 4, 5, 6 or 7 adenine (A) nucleotides, e.g., 1, 2, 3, 4, 5, 6, or 7 adenine (A) nucleotides. In embodiments, any of a) to g) above is disposed directly 3′ to the targeting domain.

In an embodiment, a sgRNA comprises, e.g., consists of, from 5′ to 3′: [targeting domain]—

(SEQ ID NO: 56)
GUUUUAGAGCUAGAAAUAGCAAGUUAAAAUAAGGCUAGUCCGUUAUCAAC
UUGAAAAAGUGGCACCGAGUCGGUGCUUUU.

In an embodiment, a sgRNA described herein comprises, e.g., consists of, from 5′ to 3′: [targeting domain]—

(SEQ ID NO: 57)
GUUUAAGAGCUAUGCUGGAAACAGCAUAGCAAGUUUAAAUAAGGCUAGUC
CGUUAUCAACUUGAAAAAGUGGCACCGAGUCGGUGCUUUU.

In embodiments, a sgRNA described herein comprises, e.g., consists of, a ribonucleic acid having the sequence:

(SEQ ID NO: 7)
NNNNNNNNNNNNNNNNNNNGUUUUAGAGCUAGAAAUAGCAAGUUAAAAUA
AGGCUAGUCCGUUAUCAACUUGAAAAAGUGGCACCGAGUCGGUGC,

where the n's refer to the residues of the targeting domain.

In an embodiment, a sgRNA described herein comprises, e.g., consists of:

(SEQ ID NO: 58)
NNNNNNNNNNNNNNNNNNNNGUUUUAGAGCUAGAAAUAGCAAGUUAAAA
UAAGGCUAGUCCGUUAUCAACUUGAAAAAGUGGCACCGAGUCGGUGCUU
UU,

where N's indicate the residues of the targeting domain, e.g., as described herein, (optionally with an inverted abasic residue at the 5′ and/or 3′ terminus).

Other exemplary sgRNA molecules and their sequences can be found in WO2017115268 and WO2018142364, the contents of which are incorporated herein.

In some embodiments, a crRNA comprises, from 5′ to 3′, preferably disposed directly 3′ to the targeting domain:

a)
(SEQ ID NO: 59)
GUUUUAGAGCUA;
b)
(SEQ ID NO: 60)
GUUUAAGAGCUA;
c)
(SEQ ID NO: 61)
GUUUUAGAGCUAUGCUG;
d)
(SEQ ID NO: 62)
GUUUAAGAGCUAUGCUG;
e)
(SEQ ID NO: 63)
GUUUUAGAGCUAUGCUGUUUUG;
f)
(SEQ ID NO: 64)
GUUUAAGAGCUAUGCUGUUUUG;
or
g)
(SEQ ID NO: 65)
GUUUUAGAGCUAUGCU.

In some embodiments, a tracr comprises, from 5′ to 3′:

a)
(SEQ ID NO: 66)
UAGCAAGUUAAAAUAAGGCUAGUCCGUUAUCAACUUGAAAAAGUGGCACC
GAGUCGGUGC;
b)
(SEQ ID NO: 67)
UAGCAAGUUUAAAUAAGGCUAGUCCGUUAUCAACUUGAAAAAGUGGCACC
GAGUCGGUGC;
c)
(SEQ ID NO: 68)
CAGCAUAGCAAGUUAAAAUAAGGCUAGUCCGUUAUCAACUUGAAAAAGUG
GCACCGAGUCGGUGC;
d)
(SEQ ID NO: 69)
CAGCAUAGCAAGUUUAAAUAAGGCUAGUCCGUUAUCAACUUGAAAAAGUG
GCACCGAGUCGGUGC;
e)
(SEQ ID NO: 70)
AACAGCAUAGCAAGUUAAAAUAAGGCUAGUCCGUUAUCAACUUGAAAAAG
UGGCACCGAGUCGGUGCUUUUUUU;
f)
(SEQ ID NO: 71)
AACAGCAUAGCAAGUUUAAAUAAGGCUAGUCCGUUAUCAACUUGAAAAAG
UGGCACCGAGUCGGUGCUUUUUUU;
g)
(SEQ ID NO: 72)
AACAGCAUAGCAAGUUAAAAUAAGGCUAGUCCGUUAUCAACUUGAAAAAG
UGGCACCGAGUCGGUGC
h)
(SEQ ID NO: 73)
GUUUAAGAGCUAUGCUGGAAACAGCAUAGCAAGUUUAAAUAAGGCUAGUC
CGUUAUCAACUUGAAAAAGUGGCACCGAGUCGGUGCUUUU;
i)
(SEQ ID NO: 74)
AGCAUAGCAAGUUAAAAUAAGGCUAGUCCGUUAUCAACUUGAAAAAGUGG
CACCGAGUCGGUGCUUU;
j)
(SEQ ID NO: 75)
GUUGGAACCAUUCAAAACAGCAUAGCAAGUUAAAAUAAGGCUAGUCCGUU
AUCAACUUGAAAAAGUGGCACCGAGUCGGUGCUUU;

k) any of a) to j), above, further comprising, at the 3′ end, at least 1, 2, 3, 4, 5, 6 or 7 uracil (U) nucleotides, e.g., 1, 2, 3, 4, 5, 6, or 7 uracil (U) nucleotides;
l) any of a) to j), above, further comprising, at the 3′ end, at least 1, 2, 3, 4, 5, 6 or 7 adenine (A) nucleotides, e.g., 1, 2, 3, 4, 5, 6, or 7 adenine (A) nucleotides; or
m) any of a) to l), above, further comprising, at the 5′ end (e.g., at the 5′ terminus), at least 1, 2, 3, 4, 5, 6 or 7 adenine (A) nucleotides, e.g., 1, 2, 3, 4, 5, 6, or 7 adenine (A) nucleotides.

In an embodiment, the sequence of k), above comprises the 3′ sequence UUUUUU, e.g., if a U6 promoter is used for transcription. In an embodiment, the sequence of k), above, comprises the 3 sequence UUUU, e.g., if an HI promoter is used for transcription. In an embodiment, sequence of k), above, comprises variable numbers of 3′ U's depending, e.g., on the termination signal of the pol-III promoter used. In an embodiment, the sequence of k), above, comprises variable 3′ sequence derived from the DNA template if a T7 promoter is used. In an embodiment, the sequence of k), above, comprises variable 3 sequence derived from the DNA template, e.g., if in vitro transcription is used to generate the RNA molecule. In an embodiment, the sequence of k), above, comprises variable 3′ sequence derived from the DNA template, e.g., if a pol-II promoter is used to drive transcription.

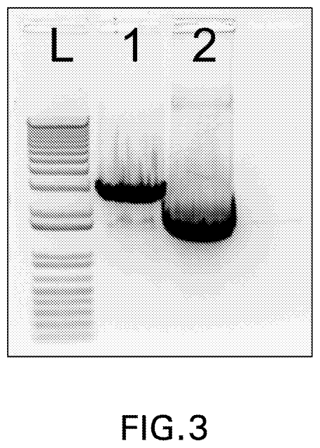

In one embodiment, the DNA template has a linearization site located after the second DNA sequence. Precise linearization at the end of second DNA sequence ensures a proper 3′ end of RNA. In one embodiment, the DNA template is a linearized DNA plasmid. See, FIG. 3. In one embodiment, the linearization site is a restriction endonuclease site, e.g., a DraI, BspQI, Sap or BbsI restriction site.

In another embodiment, the DNA template further comprises an RNA polymerase termination sequence located after the second DNA sequence and upstream from the RNA linearization site. The termination sequence is where the RNA transcript ends, but this sequence does not lead to linearization of DNA. In one embodiment, the RNA polymerase termination sequence comprises a T7 terminator sequence having a sequence with at least 90%, 95%, 98%, 99% or 100% sequence identity to SEQ ID NO: 8.

In one embodiment, the DNA template further comprises a ribozyme sequence after the second DNA sequence and upstream from the linearization sequence to ensure proper cleavage of the RNA transcript at the 3′ end. In one embodiment, the ribosome is selected from known ribozymes, such as hammerhead, hairpin, hepatitis delta virus (HDV), Varkud satellite ribozymes, etc. In one embodiment, the ribozyme is HDV and the ribozyme sequence has a sequence with at least 90%, 95%, 98%, 99% or 100% sequence identity to SEQ ID NO: 9.

In one embodiment, the DNA template further comprises an RNA polymerase termination sequence and a ribozyme sequence. In one embodiment, the ribozyme sequence is to the 3′ end of the RNA polymerase termination sequence.

In one embodiment, the DNA template further comprises an RNA polymerase promoter enhancing sequence upstream from the RNA transcription initiation site, e.g., upstream of the RNA polymerase promoter. In one embodiment, the RNA polymerase promoter enhancing sequence is a T7 RNA polymerase enhancer. In one embodiment, the T7 RNA polymerase enhancer has a sequence with at least 90%, 95%, 98%, 99% or 100% sequence identity to SEQ ID NO: 10.

In one embodiment, the linearized DNA plasmid is bound, attached or adhered to a solid support, e.g., a bead, e.g., a surface coated magnetic bead.

In another aspect, the disclosure features a DNA template for making a RNA having a length of about 20-200 bases, wherein the template is produced by a method described herein. The inventors have found that a high quality DNA template is important for generating a composition of IVT RNA transcript. In one embodiment, the composition of DNA template is a composition of linearized DNA plasmids that is substantially free from non-linear DNA plasmid template, e.g., less than 5%, 4%, 3%, 2%, 1% or no non-linear template is present in the composition. In one embodiment, the presence of non-linear DNA plasmid template is determined by any known method in the art, e.g., as determined by qPCR. In one embodiment, the presence of non-linear DNA plasmid template is determined by qPCR. In one embodiment, the composition of DNA template contains less than 3%, 2%, 1% (by weight) or no non-linear DNA plasmid template. In one embodiment, the composition of DNA template contains less than 3%, 2%, 1% (by weight) or no non-linear DNA plasmid template, e.g., as determined by qPCR. In one embodiment, the composition of DNA template contains less than 3%, 2%, 1% or no non-linear DNA plasmid template as determined by qPCR. In some embodiments, when the composition of DNA template contains more than 5% of non-linear DNA plasmid template, the composition of DNA template is linearized again until the non-linear DNA plasmid template is less than 3%, 2%, 1% or not detectable by qPCR. In one embodiment, the composition of DNA template is produced by PCR.

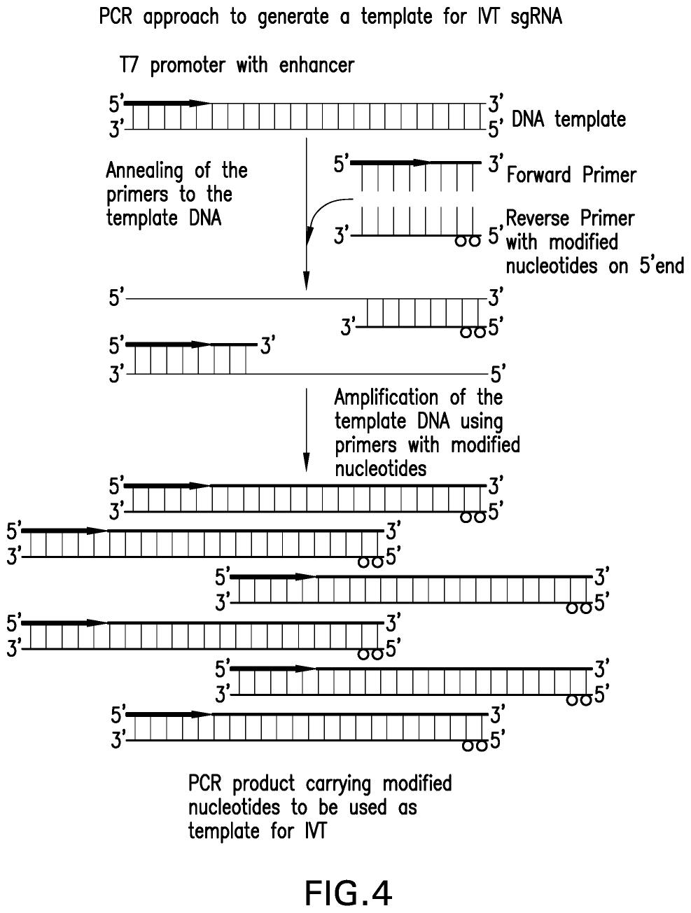

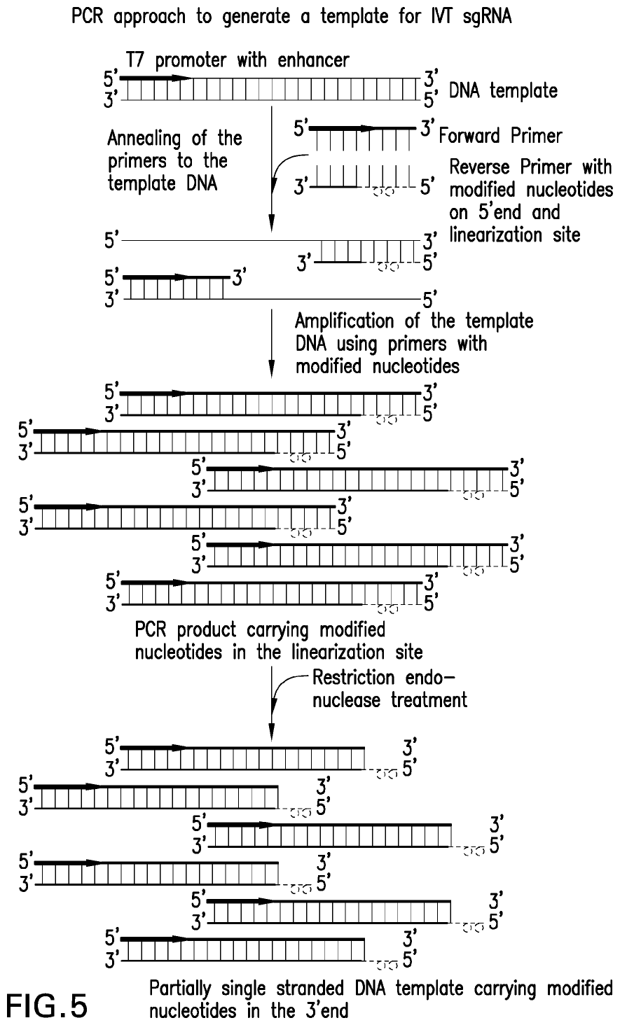

Some polymerases such as T7 polymerase are known to add non-template nucleotides on 3′-end of RNA transcript. See, Triana-Alonso et al., J. Biol. Chem. 270: 6298-6307 (1995). One way to avoid the extra nucleotide is to use chemically modified bases at the 5′-end of the antisense strand of the DNA template, which is possible when template is chemically synthesized in the form of dsDNA oligo, or partially ssDNA oligo. See, FIG. 4. See also, FIG. 5. Use of chemically modified oligonucleotides efficiently reduces addition of non-template nucleotide, e.g., n+x contaminants.

Accordingly, in one aspect, the disclosure features a DNA template for making RNA having a length of about 20-200 bases by IVT, wherein the DNA template comprises a double stranded DNA (dsDNA) template, and where the dsDNA template comprises an IVT cassette, which comprises a first DNA sequence including an RNA transcription initiation site, a polymerase promoter (e.g., an RNA polymerase promoter) upstream from an RNA transcription initiation site, an RNA sequence, and one or more (e.g., 1, 2, 3, 4, 5) modified nucleotide(s) at the 5′ end of the antisense strand of the DNA template. See, FIG. 5. In some embodiments, the modified nucleotide comprises 2′-O-alkyl modification, inverted dT or biotin. In some embodiments, the modified nucleotide is 2′-O-methyl modified nucleotide or 2′-O-(2-methoxyethyl) modified nucleotide.

In some embodiments, the RNA having a length of about 20-200 bases comprises a gRNA or a sgRNA. In some embodiments, the gRNA is about 20-150 bases in length. In some embodiments, the sgRNA is about 50-150 bases in length. In some embodiments, the sgRNA sequence encodes a fusion transcript, which comprises crRNA and optionally tracrRNA. In some embodiments, the sgRNA sequence starts with a transcription initiation nucleotide.

In one embodiment, the DNA template is a synthetic DNA template.

In one embodiment, the promoter is selected from a T7 promoter, a T3 promoter, a SP6 promoter, a Syn5 promoter, a phi 2.5 overlapping promoter, an AC15/C26 mutA promoter, an A6/B1 mutA promoter, and a phi 9 (A-15C) promoter. In one embodiment, the promoter is a T7 promoter, e.g., having a sequence with at least 90%, 95%, 98%, 99% or 100% sequence identity to SEQ ID NO: 1. In another embodiment, the promoter is a T3 promoter, e.g., having a sequence with at least 90%, 95%, 98%, 99% or 100% sequence identity to SEQ ID NO: 2. In another embodiment, the promoter is a SP6 promoter, e.g., having a sequence with at least 90%, 95%, 98%, 99% or 100% sequence identity to SEQ ID NO: 3. In another embodiment, the promoter is a Syn5 promoter, e.g., having a sequence with at least 90%, 95%, 98%, 99% or 100% sequence identity to SEQ ID NO: 4. In yet another embodiment, the promoter is a phi 2.5 overlapping promoter, e.g., having a sequence with at least 90%, 95%, 98%, 99% or 100% sequence identity to SEQ ID NO: 27. In yet another embodiment, the promoter is an AC15/C26 mutA promoter, e.g., having a sequence with at least 90%, 95%, 98%, 99% or 100% sequence identity to SEQ ID NO: 28. In yet another embodiment, the promoter is an A6/B1 mutA promoter, e.g., having a sequence with at least 90%, 95%, 98%, 99% or 100% sequence identity to SEQ ID NO: 29. In yet another embodiment, the promoter is a phi 9 (A-15C) promoter, e.g., having a sequence with at least 90%, 95%, 98%, 99% or 100% sequence identity to SEQ ID NO: 30. The nucleotide sequences of other RNA polymerase promoters (e.g., promoters for E. coli RNA polymerase) are known in the art.

In one embodiment, the RNA transcription initiation site has adenosine as the initiating nucleotide. In one embodiment, where the RNA polymerase promoter is a T7 promoter, the initiation site has adenosine as the initiating nucleotide. In another embodiment, the RNA transcription initiation site has guanosine as the initiating nucleotide. In one embodiment, where the RNA polymerase promoter is a T7 promoter, the initiation site has guanosine as the initiating nucleotide.

In one embodiment, the sgRNA sequence comprises a tracrRNA sequence having at least 90%, 95%, 98%, 99% or 100% sequence identity to SEQ ID NO: 5. In a one embodiment, the sgRNA sequence comprises a sequence having at least 90%, 95%, 98%, 99% or 100% sequence identity to SEQ ID NO: 6. In another embodiment, the sgRNA sequence comprises a sequence having at least 90%, 95%, 98%, 99% or 100% sequence identity to SEQ ID NO: 7. In one embodiment, the sgRNA sequence comprises a sequence having at least 90%, 95%, 98%, 99% or 100% sequence identity to SEQ ID NO: 33. In one embodiment, the sgRNA sequence comprises a sequence having at least 90%, 95%, 98%, 99% or 100% sequence identity to SEQ ID NO: 34. In one embodiment, the sgRNA sequence comprises a sequence having at least 90%, 95%, 98%, 99% or 100% sequence identity to SEQ ID NO: 35. In one embodiment, the sgRNA sequence comprises a sequence having at least 90%, 95%, 98%, 99% or 100% sequence identity to SEQ ID NO: 36. In one embodiment, the sgRNA sequence comprises a sequence having at least 90%, 95%, 98%, 99% or 100% sequence identity to SEQ ID NO: 37. In one embodiment, the sgRNA sequence comprises a sequence having at least 90%, 95%, 98%, 99% or 100% sequence identity to SEQ ID NO: 38. In one embodiment, the sgRNA sequence comprises a sequence having at least 90%, 95%, 98%, 99% or 100% sequence identity to SEQ ID NO: 39. In one embodiment, the sgRNA sequence comprises a sequence having at least 90%, 95%, 98%, 99% or 100% sequence identity to SEQ ID NO: 40. In one embodiment, the sgRNA sequence comprises a sequence having at least 90%, 95%, 98%, 99% or 100% sequence identity to SEQ ID NO: 41. In one embodiment, the sgRNA sequence comprises a sequence having at least 90%, 95%, 98%, 99% or 100% sequence identity to SEQ ID NO: 42. In one embodiment, the sgRNA sequence comprises a sequence having at least 90%, 95%, 98%, 99% or 100% sequence identity to SEQ ID NO: 43. In one embodiment, the sgRNA sequence comprises a sequence having at least 90%, 95%, 98%, 99% or 100% sequence identity to SEQ ID NO: 44. In one embodiment, the sgRNA sequence comprises a sequence having at least 90%, 95%, 98%, 99% or 100% sequence identity to SEQ ID NO: 45. In one embodiment, the sgRNA sequence comprises a sequence having at least 90%, 95%, 98%, 99% or 100% sequence identity to SEQ ID NO: 46. In one embodiment, the sgRNA sequence comprises a sequence having at least 90%, 95%, 98%, 99% or 100% sequence identity to SEQ ID NO: 47. In one embodiment, the sgRNA sequence comprises a sequence having at least 90%, 95%, 98%, 99% or 100% sequence identity to SEQ ID NO: 48. In one embodiment, the sgRNA sequence comprises a sequence having at least 90%, 95%, 98%, 99% or 100% sequence identity to SEQ ID NO: 49. In one embodiment, the sgRNA sequence comprises a sequence having at least 90%, 95%, 98%, 99% or 100% sequence identity to SEQ ID NO: 50. In one embodiment, the sgRNA sequence comprises a sequence having at least 90%, 95%, 98%, 99% or 100% sequence identity to SEQ ID NO: 51.

In some embodiments, the sgRNA may comprise, from 5′ to 3′, disposed 3′ to the targeting domain:

a)
(SEQ ID NO: 52)
GUUUUAGAGCUAGAAAUAGCAAGUUAAAAUAAGGCUAGUCCGUUAUCAAC
UUGAAAAAGUGGCACCGAGUCGGUGC;
b)
(SEQ ID NO: 53)
GUUUAAGAGCUAGAAAUAGCAAGUUUAAAUAAGGCUAGUCCGUUAUCAAC
UUGAAAAAGUGGCACCGAGUCGGUGC;
c)
(SEQ ID NO: 54)
GUUUUAGAGCUAUGCUGGAAACAGCAUAGCAAGUUAAAAUAAGGCUAGUC
CGUUAUCAACUUGAAAAAGUGGCACCGAGUCGGUGC;
d)
(SEQ ID NO: 55)
GUUUAAGAGCUAUGCUGGAAACAGCAUAGCAAGUUUAAAUAAGGCUAGUC
CGUUAUCAACUUGAAAAAGUGGCACCGAGUCGGUGC;

e) any of a) to d), above, further comprising, at the 3′ end, at least 1, 2, 3, 4, 5, 6 or 7 uracil (U) nucleotides, e.g., 1, 2, 3, 4, 5, 6, or 7 uracil (U) nucleotides;
f) any of a) to d), above, further comprising, at the 3′ end, at least 1, 2, 3, 4, 5, 6 or 7 adenine (A) nucleotides, e.g., 1, 2, 3, 4, 5, 6, or 7 adenine (A) nucleotides; or
g) any of a) to f), above, further comprising, at the 5′ end (e.g., at the 5′ terminus, e.g., 5′ to the targeting domain), at least 1, 2, 3, 4, 5, 6 or 7 adenine (A) nucleotides, e.g., 1, 2, 3, 4, 5, 6, or 7 adenine (A) nucleotides. In embodiments, any of a) to g) above is disposed directly 3′ to the targeting domain.

In an embodiment, a sgRNA of the invention comprises, e.g., consists of, from 5′ to 3′: [targeting domain]—

(SEQ ID NO: 56)
GUUUUAGAGCUAGAAAUAGCAAGUUAAAAUAAGGCUAGUCCGUUAUCAAC
UUGAAAAAGUGGCACCGAGUCGGUGCUUUU.

In an embodiment, a sgRNA described herein comprises, e.g., consists of, from 5′ to 3′: [targeting domain]—

(SEQ ID NO: 57)
GUUUAAGAGCUAUGCUGGAAACAGCAUAGCAAGUUUAAAUAAGGCUAGUC
CGUUAUCAACUUGAAAAAGUGGCACCGAGUCGGUGCUUUU.

In embodiments, a sgRNA described herein comprises, e.g., consists of, a ribonucleic acid having the sequence:

(SEQ ID NO: 7)
NNNNNNNNNNNNNNNNNNNGUUUUAGAGCUAGAAAUAGCAAGUUAAAAUA
AGGCUAGUCCGUUAUCAACUUGAAAAAGUGGCACCGAGUCGGUGC,

where the n's refer to the residues of the targeting domain.

In an embodiment, a sgRNA described herein comprises, e.g., consists of:

(SEQ ID NO: 58)
NNNNNNNNNNNNNNNNNNNNGUUUUAGAGCUAGAAAUAGCAAGUUAAAA
UAAGGCUAGUCCGUUAUCAACUUGAAAAAGUGGCACCGAGUCGGUGCUU
UU,

where N's indicate the residues of the targeting domain, e.g., as described herein, (optionally with an inverted abasic residue at the 5′ and/or 3′ terminus).

Other exemplary sgRNA molecules and their sequences can be found in WO2017115268 and WO2018142364, the contents of which are incorporated herein.

In some embodiments, a crRNA comprises, from 5′ to 3′, preferably disposed directly 3′ to the targeting domain:

a)
(SEQ ID NO: 59)
GUUUUAGAGCUA;
b)
(SEQ ID NO: 60)
GUUUAAGAGCUA;
c)
(SEQ ID NO: 6615)
GUUUUAGAGCUAUGCUG;
d)
(SEQ ID NO: 62)
GUUUAAGAGCUAUGCUG;
e)
(SEQ ID NO: 63)
GUUUUAGAGCUAUGCUGUUUUG;
f)
(SEQ ID NO: 64)
GUUUAAGAGCUAUGCUGUUUUG;
or
g)
(SEQ ID NO: 65)
GUUUUAGAGCUAUGCU.

In some embodiments, a tracr comprises, from 5′ to 3′:

a)
(SEQ ID NO: 66)
UAGCAAGUUAAAAUAAGGCUAGUCCGUUAUCAACUUGAAAAAGUGGCACC
GAGUCGGUGC;
b)
(SEQ ID NO: 67)
UAGCAAGUUUAAAUAAGGCUAGUCCGUUAUCAACUUGAAAAAGUGGCACC
GAGUCGGUGC;
c)
(SEQ ID NO: 68)
CAGCAUAGCAAGUUAAAAUAAGGCUAGUCCGUUAUCAACUUGAAAAAGUG
GCACCGAGUCGGUGC;
d)
(SEQ ID NO: 69)
CAGCAUAGCAAGUUUAAAUAAGGCUAGUCCGUUAUCAACUUGAAAAAGUG
GCACCGAGUCGGUGC;
e)
(SEQ ID NO: 70)
AACAGCAUAGCAAGUUAAAAUAAGGCUAGUCCGUUAUCAACUUGAAAAAG
UGGCACCGAGUCGGUGCUUUUUUU;
f)
(SEQ ID NO: 71)
AACAGCAUAGCAAGUUUAAAUAAGGCUAGUCCGUUAUCAACUUGAAAAAG
UGGCACCGAGUCGGUGCUUUUUUU;
g)
(SEQ ID NO: 72)
AACAGCAUAGCAAGUUAAAAUAAGGCUAGUCCGUUAUCAACUUGAAAAAG
UGGCACCGAGUCGGUGC
h)
(SEQ ID NO: 73)
GUUUAAGAGCUAUGCUGGAAACAGCAUAGCAAGUUUAAAUAAGGCUAGUC
CGUUAUCAACUUGAAAAAGUGGCACCGAGUCGGUGCUUUU;
i)
(SEQ ID NO: 74)
AGCAUAGCAAGUUAAAAUAAGGCUAGUCCGUUAUCAACUUGAAAAAGUGG
CACCGAGUCGGUGCUUU;
j)
(SEQ ID NO: 75)
GUUGGAACCAUUCAAAACAGCAUAGCAAGUUAAAAUAAGGCUAGUCCGUU
AUCAACUUGAAAAAGUGGCACCGAGUCGGUGCUUU;

k) any of a) to j), above, further comprising, at the 3′ end, at least 1, 2, 3, 4, 5, 6 or 7 uracil (U) nucleotides, e.g., 1, 2, 3, 4, 5, 6, or 7 uracil (U) nucleotides;
l) any of a) to j), above, further comprising, at the 3′ end, at least 1, 2, 3, 4, 5, 6 or 7 adenine (A) nucleotides, e.g., 1, 2, 3, 4, 5, 6, or 7 adenine (A) nucleotides; or
m) any of a) to l), above, further comprising, at the 5′ end (e.g., at the 5′ terminus), at least 1, 2, 3, 4, 5, 6 or 7 adenine (A) nucleotides, e.g., 1, 2, 3, 4, 5, 6, or 7 adenine (A) nucleotides.

In an embodiment, the sequence of k), above comprises the 3′ sequence UUUUUU, e.g., if a U6 promoter is used for transcription. In an embodiment, the sequence of k), above, comprises the 3 sequence UUUU, e.g., if an HI promoter is used for transcription. In an embodiment, sequence of k), above, comprises variable numbers of 3′ U's depending, e.g., on the termination signal of the pol-III promoter used. In an embodiment, the sequence of k), above, comprises variable 3′ sequence derived from the DNA template if a T7 promoter is used. In an embodiment, the sequence of k), above, comprises variable 3 sequence derived from the DNA template, e.g., if in vitro transcription is used to generate the RNA molecule. In an embodiment, the sequence of k), above, comprises variable 3′ sequence derived from the DNA template, e.g., if a pol-II promoter is used to drive transcription.

In one embodiment, the template further comprises an RNA polymerase promoter enhancing sequence upstream from the RNA transcription initiation site, e.g., upstream of the RNA polymerase promoter. In one embodiment, the RNA polymerase promoter enhancing sequence is a T7 RNA polymerase enhancer. In one embodiment, the T7 RNA polymerase enhancer has a sequence with at least 90%, 95%, 98%, 99% or 100% sequence identity to SEQ ID NO: 10.

In one embodiment, the dsDNA template is bound, attached or adhered on a solid support, e.g., a bead, e.g., a magnetic bead.

In one embodiment, the DNA template further comprises a linearization site, e.g., the modified nucleotides are part of the linearization site, e.g., a linearization site described herein, which can be used, e.g., to make a partially single stranded DNA (ssDNA) oligonucleotide, e.g., as described herein.

In another aspect, the disclosure features a DNA template for making an RNA by IVT, wherein the DNA template comprises a partially ssDNA oligonucleotide, wherein the single stranded portion of the DNA template is in the antisense strand of the DNA template and wherein the DNA template comprises an IVT cassette, which comprises a first DNA sequence including an RNA transcription initiation site, a polymerase promoter (e.g., an RNA polymerase promoter) upstream from the RNA transcription initiation site, a second DNA sequence encoding the RNA transcript having a length of about 20-200 bases disposed downstream of the RNA transcription initiation site, and one or more (e.g., 1, 2, 3, 4, 5) modified nucleotide(s) at the 5′ end of the antisense strand of the DNA template. See, e.g., FIG. 5. In some embodiments, the modified nucleotide comprises 2′-O-alkyl modification, inverted dT or biotin. In some embodiments, the modified nucleotide is 2′-O-methyl modified nucleotide or 2′-O-(2-methoxyethyl) modified nucleotide.

In some embodiments, the RNA transcript having a length of about 20-200 bases comprises a gRNA. In some embodiments, the gRNA is about 20-150 bases in length. In some embodiments, the RNA transcript having a length of about 20-200 bases comprises a sgRNA. In some embodiments, the sgRNA is about 50-150 bases in length. In some embodiments, the sgRNA sequence encodes a fusion transcript comprising crRNA and optionally tracrRNA. In some embodiments, the double stranded portion of the DNA template encodes at least a portion of the sgRNA sequence (e.g., all or a portion of the tracrRNA; a portion of the crRNA and the tracrRNA; all of the crRNA and tracrRNA). In some embodiments, the sgRNA sequence starts with a transcription initiation nucleotide that can be part of the single stranded or double stranded portion of the DNA template. In some embodiments, the RNA polymerase promoter can be part of the double stranded portion of the template. In some embodiments, all or a portion of the promoter can be part of the single stranded portion of the DNA template. The inventors have actually found that the optimal double stranded portion can be longer than previously published results. Accordingly, in some embodiments, the double stranded portion is at least 35, 40, 45, 50, 55, 60, 65, 70, 75, 80, 85, 90, 95, 100 nucleotides in length, e.g., 50, 55, 60, 65, 70, 75, 80, 85, 90 nucleotides in length.

In one embodiment, the promoter is selected from a T7 promoter, a T3 promoter, a SP6 promoter, a Syn5 promoter, a phi 2.5 overlapping promoter, an AC15/C26 mutA promoter, an A6/B1 mutA promoter, and a phi 9 (A-15C) promoter. In one embodiment, the promoter is a T7 promoter, e.g., having a sequence with at least 90%, 95%, 98%, 99% or 100% sequence identity to SEQ ID NO: 1. In one embodiment, the promoter is a T3 promoter, e.g., having a sequence with at least 90%, 95%, 98%, 99% or 100% sequence identity to SEQ ID NO: 2. In another embodiment, the promoter is a SP6 promoter, e.g., having a sequence with at least 90%, 95%, 98%, 99% or 100% sequence identity to SEQ ID NO: 3. In another embodiment, the promoter is a Syn5 promoter, e.g., having a sequence with at least 90%, 95%, 98%, 99% or 100% sequence identity to SEQ ID NO: 4. In yet another embodiment, the promoter is a phi 2.5 overlapping promoter, e.g., having a sequence with at least 90%, 95%, 98%, 99% or 100% sequence identity to SEQ ID NO: 27. In yet another embodiment, the promoter is an AC15/C26 mutA promoter, e.g., having a sequence with at least 90%, 95%, 98%, 99% or 100% sequence identity to SEQ ID NO: 28. In yet another embodiment, the promoter is an A6/B1 mutA promoter, e.g., having a sequence with at least 90%, 95%, 98%, 99% or 100% sequence identity to SEQ ID NO: 29. In yet another embodiment, the promoter is a phi 9 (A-15C) promoter, e.g., having a sequence with at least 90%, 95%, 98%, 99% or 100% sequence identity to SEQ ID NO: 30. The nucleotide sequences of other RNA polymerase promoters (e.g., promoters for E. coli RNA polymerase) are known in the art.

In one embodiment, the RNA transcription initiation site has adenosine as the initiating nucleotide. In one embodiment, where the RNA polymerase promoter is a T7 promoter, the initiation site has adenosine as the initiating nucleotide (e.g., SEQ ID NO: 20). In another embodiment, the RNA transcription initiation site has guanosine as the initiating nucleotide. In one embodiment, where the RNA polymerase promoter is a T7 promoter, the initiation site has guanosine as the initiating nucleotide (e.g., SEQ ID NO: 19).

In one embodiment, the sgRNA sequence comprises a tracrRNA sequence having at least 90%, 95%, 98%, 99% or 100% sequence identity to SEQ ID NO: 5. In one embodiment, the sgRNA sequence comprises a sequence having at least 90%, 95%, 98%, 99% or 100% sequence identity to SEQ ID NO: 6. In another embodiment, the sgRNA sequence comprises a sequence having at least 90%, 95%, 98%, 99% or 100% sequence identity to SEQ ID NO: 7. In one embodiment, the sgRNA sequence comprises a sequence having at least 90%, 95%, 98%, 99% or 100% sequence identity to SEQ ID NO: 33. In one embodiment, the sgRNA sequence comprises a sequence having at least 90%, 95%, 98%, 99% or 100% sequence identity to SEQ ID NO: 34. In one embodiment, the sgRNA sequence comprises a sequence having at least 90%, 95%, 98%, 99% or 100% sequence identity to SEQ ID NO: 35. In one embodiment, the sgRNA sequence comprises a sequence having at least 90%, 95%, 98%, 99% or 100% sequence identity to SEQ ID NO: 36. In one embodiment, the sgRNA sequence comprises a sequence having at least 90%, 95%, 98%, 99% or 100% sequence identity to SEQ ID NO: 37. In one embodiment, the sgRNA sequence comprises a sequence having at least 90%, 95%, 98%, 99% or 100% sequence identity to SEQ ID NO: 38. In one embodiment, the sgRNA sequence comprises a sequence having at least 90%, 95%, 98%, 99% or 100% sequence identity to SEQ ID NO: 39. In one embodiment, the sgRNA sequence comprises a sequence having at least 90%, 95%, 98%, 99% or 100% sequence identity to SEQ ID NO: 40. In one embodiment, the sgRNA sequence comprises a sequence having at least 90%, 95%, 98%, 99% or 100% sequence identity to SEQ ID NO: 41. In one embodiment, the sgRNA sequence comprises a sequence having at least 90%, 95%, 98%, 99% or 100% sequence identity to SEQ ID NO: 42. In one embodiment, the sgRNA sequence comprises a sequence having at least 90%, 95%, 98%, 99% or 100% sequence identity to SEQ ID NO: 43. In one embodiment, the sgRNA sequence comprises a sequence having at least 90%, 95%, 98%, 99% or 100% sequence identity to SEQ ID NO: 44. In one embodiment, the sgRNA sequence comprises a sequence having at least 90%, 95%, 98%, 99% or 100% sequence identity to SEQ ID NO: 45. In one embodiment, the sgRNA sequence comprises a sequence having at least 90%, 95%, 98%, 99% or 100% sequence identity to SEQ ID NO: 46. In one embodiment, the sgRNA sequence comprises a sequence having at least 90%, 95%, 98%, 99% or 100% sequence identity to SEQ ID NO: 47. In one embodiment, the sgRNA sequence comprises a sequence having at least 90%, 95%, 98%, 99% or 100% sequence identity to SEQ ID NO: 48. In one embodiment, the sgRNA sequence comprises a sequence having at least 90%, 95%, 98%, 99% or 100% sequence identity to SEQ ID NO: 49. In one embodiment, the sgRNA sequence comprises a sequence having at least 90%, 95%, 98%, 99% or 100% sequence identity to SEQ ID NO: 50. In one embodiment, the sgRNA sequence comprises a sequence having at least 90%, 95%, 98%, 99% or 100% sequence identity to SEQ ID NO: 51.

In some embodiments, the sgRNA may comprise, from 5′ to 3′, disposed 3′ to the targeting domain:

a)
(SEQ ID NO: 52)
GUUUUAGAGCUAGAAAUAGCAAGUUAAAAUAAGGCUAGUCCGUUAUCAAC
UUGAAAAAGUGGCACCGAGUCGGUGC;
b)
(SEQ ID NO: 53)
GUUUAAGAGCUAGAAAUAGCAAGUUUAAAUAAGGCUAGUCCGUUAUCAAC
UUGAAAAAGUGGCACCGAGUCGGUGC;
c)
(SEQ ID NO: 54)
GUUUUAGAGCUAUGCUGGAAACAGCAUAGCAAGUUAAAAUAAGGCUAGUC
CGUUAUCAACUUGAAAAAGUGGCACCGAGUCGGUGC;
d)
(SEQ ID NO: 55)
GUUUAAGAGCUAUGCUGGAAACAGCAUAGCAAGUUUAAAUAAGGCUAGUC
CGUUAUCAACUUGAAAAAGUGGCACCGAGUCGGUGC;

e) any of a) to d), above, further comprising, at the 3′ end, at least 1, 2, 3, 4, 5, 6 or 7 uracil (U) nucleotides, e.g., 1, 2, 3, 4, 5, 6, or 7 uracil (U) nucleotides;
f) any of a) to d), above, further comprising, at the 3′ end, at least 1, 2, 3, 4, 5, 6 or 7 adenine (A) nucleotides, e.g., 1, 2, 3, 4, 5, 6, or 7 adenine (A) nucleotides; or
g) any of a) to f), above, further comprising, at the 5′ end (e.g., at the 5′ terminus, e.g., 5′ to the targeting domain), at least 1, 2, 3, 4, 5, 6 or 7 adenine (A) nucleotides, e.g., 1, 2, 3, 4, 5, 6, or 7 adenine (A) nucleotides. In embodiments, any of a) to g) above is disposed directly 3′ to the targeting domain.

In an embodiment, a sgRNA of the invention comprises, e.g., consists of, from 5′ to 3′: [targeting domain]—

(SEQ ID NO: 56)
GUUUUAGAGCUAGAAAUAGCAAGUUAAAAUAAGGCUAGUCCGUUAUCAAC
UUGAAAAAGUGGCACCGAGUCGGUGCUUUU.

In an embodiment, a sgRNA described herein comprises, e.g., consists of, from 5′ to 3′: [targeting domain]—

(SEQ ID NO: 57)
GUUUAAGAGCUAUGCUGGAAACAGCAUAGCAAGUUUAAAUAAGGCUAGUC
CGUUAUCAACUUGAAAAAGUGGCACCGAGUCGGUGCUUUU.

In embodiments, a sgRNA described herein comprises, e.g., consists of, a ribonucleic acid having the sequence:

(SEQ ID NO: 7)
NNNNNNNNNNNNNNNNNNNGUUUUAGAGCUAGAAAUAGCAAGUUAAAAUA
AGGCUAGUCCGUUAUCAACUUGAAAAAGUGGCACCGAGUCGGUGC,

where the n's refer to the residues of the targeting domain.

In an embodiment, a sgRNA described herein comprises, e.g., consists of:

(SEQ ID NO: 58)
NNNNNNNNNNNNNNNNNNNNGUUUUAGAGCUAGAAAUAGCAAGUUAAAA
UAAGGCUAGUCCGUUAUCAACUUGAAAAAGUGGCACCGAGUCGGUGCUU
UU,

where m indicates a base with 2′O-Methyl modification, * indicates a phosphorothioate bond, and N's indicate the residues of the targeting domain, e.g., as described herein, (optionally with an inverted abasic residue at the 5′ and/or 3′ terminus).

Other exemplary sgRNA molecules and their sequences can be found in WO2017115268 and WO2018142364, the contents of which are incorporated herein.

In some embodiments, a crRNA comprises, from 5′ to 3′, preferably disposed directly 3′ to the targeting domain:

a)
(SEQ ID NO: 59)
GUUUUAGAGCUA;
b)
(SEQ ID NO: 60)
GUUUAAGAGCUA;
c)
(SEQ ID NO: 61)
GUUUUAGAGCUAUGCUG;
d)
(SEQ ID NO: 62)
GUUUAAGAGCUAUGCUG;
e)
(SEQ ID NO: 63)
GUUUUAGAGCUAUGCUGUUUUG;
f)
(SEQ ID NO: 64)
GUUUAAGAGCUAUGCUGUUUUG;
or
g)
(SEQ ID NO: 65)
GUUUUAGAGCUAUGCU.

In some embodiments, a tracr comprises, from 5′ to 3′:

a)
(SEQ ID NO: 66)
UAGCAAGUUAAAAUAAGGCUAGUCCGUUAUCAACUUGAAAAAGUGGCACC
GAGUCGGUGC;
b)
(SEQ ID NO: 67)
UAGCAAGUUUAAAUAAGGCUAGUCCGUUAUCAACUUGAAAAAGUGGCACC
GAGUCGGUGC;
c)
(SEQ ID NO: 68)
CAGCAUAGCAAGUUAAAAUAAGGCUAGUCCGUUAUCAACUUGAAAAAGUG
GCACCGAGUCGGUGC;
d)
(SEQ ID NO: 69)
CAGCAUAGCAAGUUUAAAUAAGGCUAGUCCGUUAUCAACUUGAAAAAGUG
GCACCGAGUCGGUGC;
e)
(SEQ ID NO: 70)
AACAGCAUAGCAAGUUAAAAUAAGGCUAGUCCGUUAUCAACUUGAAAAAG
UGGCACCGAGUCGGUGCUUUUUUU;
f)
(SEQ ID NO: 71)
AACAGCAUAGCAAGUUUAAAUAAGGCUAGUCCGUUAUCAACUUGAAAAAG
UGGCACCGAGUCGGUGCUUUUUUU;
g)
(SEQ ID NO: 72)
AACAGCAUAGCAAGUUAAAAUAAGGCUAGUCCGUUAUCAACUUGAAAAAG
UGGCACCGAGUCGGUGC
h)
(SEQ ID NO: 73)
GUUUAAGAGCUAUGCUGGAAACAGCAUAGCAAGUUUAAAUAAGGCUAGUC
CGUUAUCAACUUGAAAAAGUGGCACCGAGUCGGUGCUUUU;
i)
(SEQ ID NO: 74)
AGCAUAGCAAGUUAAAAUAAGGCUAGUCCGUUAUCAACUUGAAAAAGUGG
CACCGAGUCGGUGCUUU;
j)
(SEQ ID NO: 75)
GUUGGAACCAUUCAAAACAGCAUAGCAAGUUAAAAUAAGGCUAGUCCGUU
AUCAACUUGAAAAAGUGGCACCGAGUCGGUGCUUU;

k) any of a) to j), above, further comprising, at the 3′ end, at least 1, 2, 3, 4, 5, 6 or 7 uracil (U) nucleotides, e.g., 1, 2, 3, 4, 5, 6, or 7 uracil (U) nucleotides;
l) any of a) to j), above, further comprising, at the 3′ end, at least 1, 2, 3, 4, 5, 6 or 7 adenine (A) nucleotides, e.g., 1, 2, 3, 4, 5, 6, or 7 adenine (A) nucleotides; or
m) any of a) to l), above, further comprising, at the 5′ end (e.g., at the 5′ terminus), at least 1, 2, 3, 4, 5, 6 or 7 adenine (A) nucleotides, e.g., 1, 2, 3, 4, 5, 6, or 7 adenine (A) nucleotides.

In an embodiment, the sequence of k), above comprises the 3′ sequence UUUUUU, e.g., if a U6 promoter is used for transcription. In an embodiment, the sequence of k), above, comprises the 3 sequence UUUU, e.g., if an HI promoter is used for transcription. In an embodiment, sequence of k), above, comprises variable numbers of 3′ U's depending, e.g., on the termination signal of the pol-III promoter used. In an embodiment, the sequence of k), above, comprises variable 3′ sequence derived from the DNA template if a T7 promoter is used. In an embodiment, the sequence of k), above, comprises variable 3 sequence derived from the DNA template, e.g., if in vitro transcription is used to generate the RNA molecule. In an embodiment, the sequence of k), above, comprises variable 3′ sequence derived from the DNA template, e.g., if a pol-II promoter is used to drive transcription.

In one embodiment, the template further comprises an RNA polymerase promoter enhancing sequence upstream from the RNA transcription initiation site, e.g., upstream of the RNA polymerase promoter. In one embodiment, the RNA polymerase promoter enhancing sequence is a T7 RNA polymerase enhancer. In one embodiment, the T7 RNA polymerase enhancer has a sequence with at least 90%, 95%, 98%, 99% or 100% sequence identity to SEQ ID NO: 10. In one embodiment, all or part of the RNA polymerase enhancing sequence is part of the double stranded portion of the DNA template. In some embodiments, all or part of the RNA polymerase enhancing sequence is part of the single stranded portion of the DNA template.

In some embodiments, the modified nucleotide comprises 2′-O-alkyl modification, inverted dT or biotin. In some embodiments, the modified nucleotide is 2′-O-methyl modified nucleotide or 2′-O-(2-methoxyethyl) modified nucleotide.

In one embodiment, the partially ssDNA is bound, attached or adhered on a solid support, e.g., a bead, e.g., a magnetic bead.

In another aspect, the disclosure features a method of making a RNA having a length of about 20-200 bases by in vitro transcription (IVT), comprising the steps of obtaining a template for making a RNA selected from the group of DNA templates described herein, and then producing an RNA transcript by in vitro transcription of the DNA template. An advantage of the disclosed method is that the IVT-made RNA transcript described herein has improved integrity (i.e., sequence identity) (such as in the crRNA sequence (˜100%)), with no observable n−x variants or n+1 variant in the RNA transcripts (such as in the crRNA sequence). This reduces the off-target effects previously observed with CRISPR techniques, which can be due to errors on the synthesis of crRNA. In some embodiments, the IVT-made RNA transcript having a length of about 20-200 bases comprises a gRNA. In some embodiments, the gRNA is about 20-150 bases in length. In some embodiments, the IVT-made RNA transcript having a length of about 20-200 bases comprises a sgRNA. In some embodiments, the sgRNA is about 50-150 bases in length.

In one embodiment, the method advantageously provides a sgRNA product with no observable n−x or n+x (e.g., n+1) variants in the crRNA region, e.g., as determined by LC-MS.

In one embodiment, the composition of IVT-made RNA transcript having a length of about 20-200 bases is not treated with DNase, e.g., the method results in a composition of IVT-made RNA transcript having a length of about 20-200 bases that is free of DNase and/or DNase associated impurities, e.g., DNA pieces, e.g., pieces of DNA template that are 10 or less nucleotides in length, e.g., 4, 3, 2 or 1 nucleotides in length.

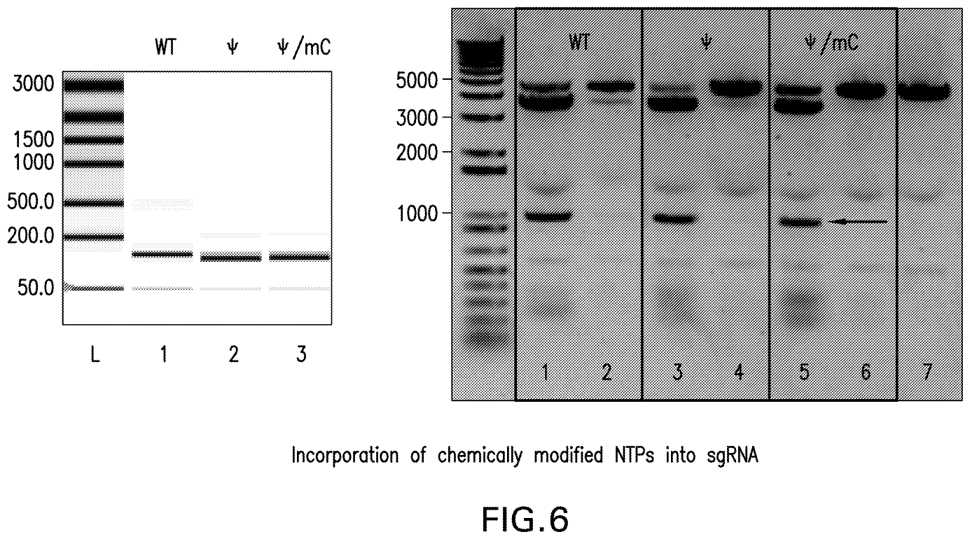

In one embodiment, the in vitro synthesized RNA can contain a modified nucleotide. As described herein, the in vitro synthesized RNA can contain a modified nucleotide selected from one or more of the nucleotides provided herein, including those described in U.S. Pat. No. 8,278,036 (Karikó et al.); U.S. Pat. Appl. No. 2013/0102034 (Schrum); U.S. Pat. Appl. No. 2013/0115272 (deFougerolles et al.) and U.S. Pat. Appl. No. 2013/0123481 (deFougerolles et al.). In one embodiment, the method advantageously minimizes the immunogenicity and enhances the stability of the final product, e.g., IVT-made RNA transcript having a length of about 20-200 bases, e.g., sgRNA, by incorporating chemical modifications into the RNA during in vitro transcription. In one embodiment, pseudouridine (ψ) is incorporated in vitro into the RNA transcript. In one embodiment, 5-methylcytidine (m⁵C) is incorporated in vitro into the RNA transcript. In one embodiment, both ψ and m⁵C are incorporated into the in vitro RNA transcript. In one embodiment, other modified nucleotides are incorporated into the RNA transcript. FIG. 6 shows a comparison of in vitro transcribed RNA using either natural or chemically modified sgRNAs. Incorporation of pseudouridine (ψ), or combination of pseudouridine (ψ) and 5-methylcytidine (m⁵C) into the in vitro sgRNA transcript does not affect activity of sgRNA in an in vitro Cas9 assay. In one embodiment, all “A” nucleotides of the IVT-made RNA (e.g., IVT-made sgRNA) are the same modified nucleotides. In one embodiment, all “U” nucleotides of the IVT-made RNA (e.g., IVT-made sgRNA) are the same modified nucleotides. In one embodiment, all “G” nucleotides of the IVT-made RNA (e.g., IVT-made sgRNA) are the same modified nucleotides. In one embodiment, all “C” nucleotides of the IVT-made RNA (e.g., IVT-made sgRNA) are the same modified nucleotides.

In one embodiment, the method provides a sgRNA transcript with a total length of from 50 mer-120 mer (e.g., 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119 or 120 mer).

In one embodiment, the IVT-made RNA transcript having a length of about 20-200 bases, e.g., sgRNA, is capped, thereby enhancing nuclease stability of the 5′ end of the RNA and at the same time reducing immunogenicity. The inventors have performed experiments that indicate that a 5′ cap is compatible with CRISPR activity. In one embodiment, the cap can be an ARCA, a thio-ARCA or a chemical cap, e.g., such as described in WO 2016/098028 A1. See, EXAMPLE 4.

In another aspect, the disclosure features a method of making RNA transcript having a length of about 20-200 bases by in vitro transcription (IVT) for industrial-scale production. In one embodiment, at least 0.5 to 1 g of RNA is made by the industrial-scale process. The RNA transcript produced by the steps of providing a composition of linearized DNA plasmid template, e.g., one of the DNA plasmid templates described herein, purifying the linearized DNA template on an industrial scale, and then producing a composition of RNA transcript by in vitro transcription of the linearized DNA template on an industrial scale. In some embodiments, the RNA transcript having a length of about 20-200 bases comprises a gRNA. In some embodiments, the gRNA is about 20-150 bases in length. In some embodiments, the RNA transcript having a length of about 20-200 bases comprises a sgRNA. In some embodiments, the sgRNA is about 50-150 bases in length.

In one embodiment, the method further includes a step of purifying a composition of RNA transcript (e.g., gRNA or sgRNA), where a DNase treatment step is not included the purification process. DNase produces 1-4 nucleotide-long stretches of free DNA that can remain in solution, even after lithium chloride precipitation. These small pieces of DNA can then hybridize to the full-length RNA and interfere with the CRISPR reactions. Because of this heterogeneity and the risk that it can cause or contribute to immunogenicity of the RNA preparation, the inventors recognized a better purification method. By omitting the DNase digestion step, the full-length DNA template remains in solution during purification and the presence of residual DNA contaminants is eliminated.

In one embodiment, the method further includes a step of amplifying (e.g., for quality control purpose) the DNA template by qPCR.

In one embodiment, the method further includes a step of purifying a RNA transcript (e.g., gRNA or sgRNA) by HPLC, e.g., reverse phase HPLC. Those of skill in the biotechnological arts can use the purification method to separate RNA transcript having a length of about 20-200 bases from full-length DNA, as well as separating RNA transcript having a length of about 20-200 bases from as other immune stimulating moieties, e.g., as shown in TABLE 3.

In one embodiment, the purified RNA transcript is tested for the presence of immune stimulating moieties, by an immunogenicity assay. In one embodiment, the immunogenicity assay is a THP-1 monocytic cell line-based immunogenicity assay.

In one embodiment, the produced RNA transcript is substantially free of any immune stimulating moieties. In one embodiment, the produced RNA transcript is substantially free of RNA transcripts having n+x variants. In one embodiment, the produced RNA transcript is substantially free of RNA transcripts having n−x variants.

As described above, the methods described herein provide solutions to some of the problems of chemical synthesis and other problems known in the art. In some embodiments, the methods described herein produce a composition of polynucleotides (e.g., gRNA, sgRNA) having less than 6%, 5%, 4%, 3%, 2%, 1% or no detectable n+x or n−x variants, preferably less than 4%, 3%, 2%, 1% or no detectable n+x or n−x variants. In some embodiments, the methods described herein produce a composition of polynucleotides (e.g., IVT-made RNA transcript having a length of about 20-200 bases, gRNA, sgRNA) having less than 6%, 5%, 4%, 3%, 2%, 1% or no detectable DNase and/or DNase associated impurities (e.g., DNA pieces, e.g., pieces of DNA template that are 10 or less nucleotides in length, e.g., 4, 3, 2 or 1 nucleotides in length). In some embodiments, the methods described herein produce a composition of polynucleotides (e.g., IVT-made RNA transcript having a length of about 20-200 bases, gRNA, sgRNA) having purity that is at least 50%, 55%, 60%, 65%, 70%, 75%, 80%, 85%, 90%, 95%, 100%, 150%, 200% or higher than the purity of the chemically synthesized product. In some embodiments, the methods described herein provide better batch-to-batch reproducibility compared to other synthesis methods, e.g., chemical synthesis, partially due to less impurities and/or more consistent impurities of the composition of polynucleotides (e.g., IVT-made RNA transcript having a length of about 20-200 bases, gRNA, sgRNA) generated by the methods described herein. In some embodiments, the methods described herein are more cost efficient than other synthesis methods, e.g., chemical synthesis. In some embodiments, the methods described herein have advantages of preparing longer gRNA and/or sgRNA sequences. Typically, chemically synthesis can handle polynucleotides having 60 nt or less. In some embodiments, the composition (e.g., IVT-made RNA transcript having a length of about 20-200 bases, gRNA, sgRNA) prepared according to the methods/processes described herein have higher biological activity compared that prepared by chemical synthesis (see, e.g., FIG. 15). In some embodiments, the methods described herein produce gRNA or sgRNA having modified nucleotides (see, Example 9).

In another aspect, the disclosure features a composition of RNA transcript that has been produced by a process described herein, where a DNase treatment step is not included the purification process and where the RNA transcript is about 20-200 bases in length. In some embodiments, the RNA transcript having a length of about 20-200 bases comprises a gRNA. In some embodiments, the gRNA is about 20-150 bases in length. In some embodiments, the RNA transcript having a length of about 20-200 bases comprises a sgRNA. In some embodiments, the sgRNA is about 50-150 bases in length. In one embodiment, the composition of RNA transcript has been purified by reverse-phase HPLC. Appropriate purification methods and analytical assays are used to monitor the purity of the generated RNA products, including qPCR to determine residual DNA plasmid and negative strand, J2 dot blot to monitor dsRNA products and other methods.

In one embodiment, the composition of RNA product produced by the methods described herein has a homogeneity that is higher than a corresponding composition of RNA produced by chemical synthesis. Compared to chemical synthesis, the composition of IVT RNA product has a higher purity and the production process allows for higher batch-to-batch reproducibility. In one embodiment, the disclosure features a more homogenous composition of in vitro transcribed RNA transcript compared to chemically synthesized compositions of in vitro transcribed RNA transcripts, with a reduced amount of n−x product (e.g., the composition of RNA that has less than 5%, 4%, 3%, 2% or 1% n−x RNA product). In one embodiment, the composition of in vitro transcribed RNA is substantially free of DNase and/or DNase associated impurities, e.g., less than 3%, 2%, 1% or no residual DNA pieces are in the composition.

In one embodiment, the composition of RNA transcript includes one or more modified nucleotides. In one embodiment, the composition of RNA transcript includes at least one pseudouridine (P), at least one 5-methylcytidine (m⁵C) or both.

In one embodiment, the composition of RNA transcript is dephosphorylated and/or capped at the 5′ end, at the 3′ end, or at both the 5′ end and 3′ end. In one embodiment, the composition of RNA transcript is dephosphorylated at the 5′ end, at the 3′ end, or at both the 5′ end and 3′ end. In one embodiment, the composition of RNA transcript is capped at the 5′ end, at the 3′ end, or at both the 5′ end and 3′ end.

In one embodiment, the IVT-made RNA transcript (e.g., sgRNA) in the composition is coupled to a Cas9 protein, e.g., a Cas9 protein described herein, or a Cpf1 protein, e.g., a Cpf1 protein described herein.

In another aspect, the disclosure features a pharmaceutical composition, comprising a RNA transcript product described herein, e.g., a RNA transcript that has been produced by a process described herein, and a pharmaceutically acceptable carrier.

In one aspect, described herein is a composition comprising an IVT-made polynucleotide having a length of about 20-200 bases, where the composition is substantially free of immune stimulating moieties and/or substantially free of n−1 and/or n+1 variants.

In one embodiment, the IVT-made polynucleotide has a length of about 50-150 bases. In one embodiment, the IVT-made polynucleotide has a length of about 60-150 bases. In one embodiment, the IVT-made polynucleotide has a length of about 50-120 bases. In one embodiment, the IVT-made polynucleotide has a length of about 60-120 bases. In one embodiment, the IVT-made polynucleotide has a length of about 75-120 bases.

In one embodiment, the IVT-made polynucleotide includes pseudouridine (ψ), or 5-methylcytidine (m⁵C), or both ψ and m⁵C.

In one embodiment, the IVT-made polynucleotide is about 50 bases to 150 bases in length. In one embodiment, the IVT-made polynucleotide is a sgRNA sequence. In one embodiment, the sgRNA sequence is about 50 bases to 120 bases in length.

In one embodiment, the IVT-made polynucleotide is dephosphorylated and/or capped at the 5′ end, at the 3′ end, or at both the 5′ end and 3′ end. In one embodiment, the IVT-made polynucleotide is dephosphorylated at the 5′ end, at the 3′ end, or at both the 5′ end and 3′ end. In one embodiment, the IVT-made polynucleotide is capped at the 5′ end, at the 3′ end, or at both the 5′ end and 3′ end.

In another aspect, the disclosure features a method of determining whether a sgRNA was produced by in vitro transcription. A determination that an sgRNA has a homogeneity (e.g., only n+x transcripts) that is higher than from a corresponding chemical synthesis of the sgRNA product (e.g., both n+x transcripts and n−x transcripts) will lead one of skill in the art to a conclusion that the sgRNA transcript was produced by IVT.

In another aspect, described herein is a cell comprising a composition of RNA transcript that has been produced by a process described herein. In some embodiments, the cell further comprises an RNA-guided DNA endonuclease enzyme (such as Cas9).

In another aspect, the disclosure features a method of altering gene expression in a cell, by introducing into the cell a composition described herein (e.g., a sgRNA or gRNA transcript described herein).

In one embodiment, the method further includes a step of introducing to the cell an RNA-guided DNA endonuclease enzyme. In one embodiment, the RNA-guided DNA endonuclease enzyme is Cas9, Cpf1 or a class II CRISPR endonuclease or a variant thereof.

In one embodiment, the cell is an animal cell. In one embodiment, the cell is a mammalian, primate or human cell. In one embodiment, the cell is a hematopoietic stem or progenitor cell (HSPC).

In one aspect, described herein is a cell that is altered by the method described herein.

In one aspect, described herein is a cell obtained by the method described herein.

In one aspect, provided herein is the IVT-made RNA transcript or the composition or the pharmaceutical composition described herein for use in altering gene expression in a cell.

### Modified RNA

“Modified” means a changed state or structure of a molecule. A “modified” mRNA contains ribonucleosides that encompass modifications relative to the standard guanine (G), adenine (A), cytidine (C), and uridine (U) nucleosides. The nonstandard nucleosides can be naturally occurring or non-naturally occurring. RNA can be modified in many ways including chemically, structurally, and functionally, by methods known to those of skill in the biotechnological arts. Such RNA modifications can include, e.g., modifications normally introduced post-transcriptionally to mammalian cell mRNA. Moreover, RNA molecules can be modified by the introduction during transcription of natural and non-natural nucleosides or nucleotides, as described in U.S. Pat. No. 8,278,036 (Karikó et al.); U.S. Pat. Appl. No. 2013/0102034 (Schrum); U.S. Pat. Appl. No. 2013/0115272 (deFougerolles et al.) and U.S. Pat. Appl. No. 2013/0123481 (deFougerolles et al.). For examples of incorporation of ψ (pseudouridine) or m⁵C (5-methylcytidine) into mRNA, see, U.S. Pat. No. 8,278,036 (Karikó et al.); WO 2015/095351 (Novartis AG); Karikó K et al. Curr. Opin. Drug Disc. Devel. 10(5): 523-532 (2007); Karikó K et al. Mol. Therap. 16(11): 1833-1840 (2008) and Anderson B R et al., Nucleic Acids Res. 38(17): 5884-5892 (2010).

The in vitro synthesized RNA can contain modified nucleotides selected from the following: ψ (pseudouridine); m⁵C (5-methylcytidine); m⁵U (5-methyluridine); m⁶A (N⁶-methyladenosine); s²U (2-thiouridine); Um (2′-O-methyl-U; 2′-O-methyluridine); m¹A (1-methyladenosine); m²A (2-methyladenosine); Am (2′-O-methyladenosine); ms²m⁶A (2-methylthio-N⁶-methyladenosine); i⁶A (N⁶-isopentenyladenosine); ms²i⁶A (2-methylthio-N⁶isopentenyladenosine); io⁶A (N⁶-(cis-hydroxyisopentenyl)adenosine); ms²i⁶A (2-methylthio-N⁶-(cis-hydroxyisopentenyl)adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A(N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C(N⁴-acetylcytidine); f⁵C (5-formylcytidine); m⁵ Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m² 2G (N²,N²-dimethylguanosine); m²Gm (N²,2′-O-dimethylguanosine); m²²Gm (N²,N²,2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQo (7-cyano-7-deazaguanosine); preQ₁ (7-aminomethyl-7-deazaguanosine); G* (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵s²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶ 2A (N⁶,N⁶-dimethyladenosine); Im (2′-O-methylinosine); m⁴C(N⁴-methylcytidine); m⁴ Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶ 2Am (N⁶,N⁶,O-2′-trimethyladenosine); m²,7G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); Tm⁵U (5-taurinomethyluridine); Tm⁵s²U (5-taurinomethyl-2-thiouridine)); imG-14 (4-demethylwyosine); imG2 (isowyosine); andac⁶A (N⁶-acetyladenosine). The sgRNA can include any combination of modified nucleotides, e.g., the modified nucleotides described herein.

In an embodiment, modified nucleotides, e.g., nucleotides having modifications as described herein, can be incorporated into a nucleic acid, e.g., a “modified nucleic acid.” In some embodiments, the modified nucleic acids comprise one, two, three or more modified nucleotides. In some embodiments, at least 5% (e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%) of the positions in a modified nucleic acid are modified nucleotides.

### Cas9 Molecules

In one embodiment, the sgRNA described herein is associated with a Cas9 molecule, e.g., a Cas9 molecule described herein. Cas9 molecules can be from, e.g., Streptococcus pyogenes, Streptococcus thermophilus, Staphylococcus aureus or Neisseria meningitides. See, e.g., Horvath et al. (2010) Science 327(5962): 167-170, and Deveau et al. (2008) J. Bacteriol. 190(4): 1390-1400. An active Cas9 molecule of Staphylococcus aureus is described by Ran et al. (2015) Nature 520: 186-191. An active Cas9 molecule of Neisseria meningitides is described by Hou et al. (2013) PNAS Early Edition 1-6. The ability of a Cas9 molecule to recognize a PAM sequence can be determined, e.g., using a transformation assay described in Jinek et al. (2012) Science 337: 816.

A Cas9 molecule can also be a protein having an amino acid sequence with homology to any Cas9 molecule sequence described herein or to a naturally occurring Cas9 molecule sequence, e.g., from a species listed herein or described in Chylinski et al. (2013) RNA Biology 10: 5,‘I2’I-T; Hou et al. (2013) PNAS Early Edition 1-6. A Cas9 molecule can also be a Streptococcus pyogenes Cas9 variant, such as a variant described in Slaymaker et al. (2015) Science Express, at Science DOI: 10.1126/science.aad5227; Kleinstiver et al. (2016) Nature 529, 490-495, at doi: 10.1038/nature16526; or US 2016/0102324. The Cas9 molecule can be a chimeric Cas9 molecule, described in, e.g., U.S. Pat. Nos. 8,889,356, 8,889,418, 8,932,814, 9,322,037, 9,388,430 and 9,267,135; U.S. Patent Publications US 2015/0118216, US 2014/0295556 and US 2016/153003; and PCT Patent Publications WO 2014/152432, WO 2015/089406, WO 2015/006294, WO 2016/022363, WO 2016/057961, WO 2016/106244, and WO 2016/131009. The Cas9 molecule, e.g., a Cas9 of Streptoccocus pyogenes, can additionally comprise one or more amino acid sequences that confer additional activity. See, e.g., Sorokin (2007) Biochemistry (Moscow) 72: 13, 1439-1457; Lange (2007) J. Biol. Chem. 282: 8, 5101-5).

Functional Analysis of sgRNA

sgRNA and Cas9/sgRNA complexes can be evaluated by methods known to those of skill in the art. Exemplary methods for evaluating the endonuclease activity of Cas9 molecule have been described previously, e.g., by Jinek et al. (2012) Science 337: 816-821.

Binding and Cleavage Assay: Testing the endonuclease activity of Cas9 molecule: The ability of a Cas9 molecule/gRNA molecule complex to bind to and cleave a target nucleic acid can be evaluated in a plasmid cleavage assay. In this assay, synthetic or in vitro-transcribed gRNA molecule is pre-annealed prior to the reaction by heating to 95° C. and slowly cooling down to room temperature. Native or restriction digest-linearized plasmid DNA (300 ng (˜8 nM)) is incubated for 60 min at 37° C. with purified Cas9 protein molecule (50-500 nM) and gRNA (50-500 nM, 1:1) in a Cas9 plasmid cleavage buffer (20 mM HEPES pH 7.5, 150 mM KCl, 0.5 mM DTT, 0.1 mM EDTA) with or without 10 mM MgCl₂. The reactions are stopped with 5×DNA loading buffer (30% glycerol, 1.2% SDS, 250 mM EDTA), resolved by a 0.8 or 1% agarose gel electrophoresis and visualized by ethidium bromide staining. The resulting cleavage products indicate whether the Cas9 molecule cleaves both DNA strands, or only one of the two strands. Linear DNA products indicate the cleavage of both DNA strands. Nicked open circular products indicate that only one of the two strands is cleaved.

Alternatively, the ability of a Cas9 molecule/gRNA molecule complex to bind to and cleave a target nucleic acid can be evaluated in an oligonucleotide DNA cleavage assay. In this assay, DNA oligonucleotides (10 pmol) are radiolabeled by incubating with 5 units T4 polynucleotide kinase and −3-6 pmol (˜20-40 mCi) [γ-32P]-ATP in IX T4 polynucleotide kinase reaction buffer at 37° C. for 30 m, in a 50 μL reaction. After heat inactivation (65° C. for 20 min), reactions are purified through a column to remove unincorporated label. Duplex substrates (100 nM) are generated by annealing labeled oligonucleotides with equimolar amounts of unlabeled complementary oligonucleotide at 95° C. for 3 min, followed by slow cooling to room temperature. For cleavage assays, gRNA molecules are annealed by heating to 95° C. for 30 s, followed by slow cooling to room temperature. Cas9 (500 nM final concentration) is pre-incubated with the annealed gRNA molecules (500 nM) in cleavage assay buffer (20 mM HEPES pH 7.5, 100 mM KCl, 5 mM MgC12, 1 mM DTT, 5% glycerol) in a total volume of 9 μl. Reactions are initiated by the addition of 1 μl target DNA (10 nM) and incubated for 1 hr at 37° C. Reactions are quenched by the addition of 20 μl of loading dye (5 mM EDTA, 0.025% SDS, 5% glycerol in formamide) and heated to 95° C. for 5 min. Cleavage products are resolved on 12% denaturing polyacrylamide gels containing 7 M urea and visualized by phosphorimaging. The resulting cleavage products indicate that whether the complementary strand, the non-complementary strand, or both, are cleaved.

One or both of these assays can be used to evaluate the suitability of a candidate gRNA molecule or candidate Cas9 molecule.

Surveyor assay. The components of a CRISPR system sufficient to form a CRISPR complex, including the guide sequence to be tested, can be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence can be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will be understood by those of skill in the biotechnological art. A guide sequence can be selected to target any target sequence. The target sequence can be a sequence within a genome of a cell. Exemplary target sequences include those that are unique in the target genome. One of skill in the biotechnological arts can select a guide sequence to reduce the degree secondary structure within the guide sequence, e.g., about or less than about 75%, 50%, 40%, 30%, 25%, 20%, 15%, 10%, 5%, 1%, or fewer of the nucleotides of the guide sequence participate in self-complementary base pairing when optimally folded. Optimal folding can be determined by any suitable polynucleotide folding algorithm. Some programs are based on calculating the minimal Gibbs free energy. An example of one such algorithm is mFold, as described by Zuker & Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm. See e.g. Gruber et al. (2008) Cell 106(1): 23-24; and Carr & Church (2009) Nature Biotechnol. 27(12): 1 151-62.

### Pharmaceutical Compositions

Pharmaceutical compositions described herein may comprise a IVT-made RNA molecule described herein, e.g., a plurality of sgRNA or gRNA molecules as described herein, or a cell (e.g., a population of cells, e.g., a population of hematopoietic stem cells) comprising one or more cells modified with one or more sgRNA or gRNA molecules described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

In one embodiment, the pharmaceutical composition is substantially free of, e.g., there are no detectable levels of a contaminant, e.g., selected from the group consisting of endotoxin, mycoplasma, mouse antibodies, pooled human serum, bovine serum albumin, bovine serum, culture media components, unwanted CRISPR system components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

### ADDITIONAL EMBODIMENTS

Embodiment 1. A DNA template (an IVT cassette) for making a single guide ribonucleic acid (sgRNA) transcript, comprising

(a) an sgRNA sequence comprising an sgRNA transcription initiation site;
(b) a polymerase promoter upstream from the sgRNA transcription initiation site; and
(c) a linearization site downstream from the sgRNA sequence.

Embodiment 2. The DNA template of embodiment 1, wherein the template is part of a DNA plasmid.

Embodiment 3. The DNA template of embodiment 1, wherein the polymerase promoter is selected from the group consisting of T7 polymerase promoter, a T3 polymerase promoter, an SP6 polymerase promoter, a Syn5 polymerase promoter, and an E. coli RNase promoter.

Embodiment 4. The DNA template of embodiment 1, wherein the linearization site is a restriction endonuclease site.

Embodiment 5. The DNA template of embodiment 4, wherein the restriction endonuclease site is selected from the group consisting of DraI, BspQI, SapI and BbsI.

Embodiment 6. The DNA template of embodiment 1, wherein the DNA template has been linearized.

Embodiment 7. The DNA template of embodiment 1, further comprising a ribozyme sequence, e.g., downstream from the sgRNA sequence and upstream of the linearization site.

Embodiment 8. The DNA template of embodiment 7, wherein the ribozyme sequence is selected from the group consisting of hammerhead, hairpin, hepatitis delta virus and Varkud satellite ribozyme.

Embodiment 9. The DNA template of embodiment 1, further comprising a T7 terminator sequence, e.g., downstream from the sgRNA sequence and upstream of the linearization site.

Embodiment 10. The DNA template of embodiment 1, further comprising a promoter enhancing sequence upstream from the sgRNA transcription initiation site.

Embodiment 11. A double stranded DNA (dsDNA) template for making a single guide ribonucleic acid (sgRNA) transcript, comprising

(a) a sgRNA sequence comprising a sgRNA transcription initiation site;
(b) a polymerase promoter upstream from the sgRNA transcription initiation site, and
(c) one or more modified nucleotides at the 5′ end of the antisense strand of the dsDNA template.

Embodiment 12. The dsDNA template of embodiment 11, comprising a transcriptional enhancer sequence upstream of the polymerase promoter.

Embodiment 13. The dsDNA template of embodiment 11, wherein the one or more modified nucleotide is 2′-O-methyl modified nucleotide.

Embodiment 14. The dsDNA template of embodiment 11, wherein the polymerase promoter is selected from the group consisting of T7 polymerase promoter, a T3 polymerase promoter, an SP6 polymerase promoter, a Syn5 polymerase promoter, and an E. coli RNase promoter.

Embodiment 15. The dsDNA template of embodiment 11, wherein the linearization site is a restriction endonuclease site.

Embodiment 16. The dsDNA template of embodiment 11, wherein the restriction endonuclease site is selected from the group consisting of DraI, BspQI, SapI and BbsI.

Embodiment 17. A partially single stranded DNA (ssDNA) template for making a single guide ribonucleic acid (sgRNA) transcript, comprising

(a) a sgRNA sequence comprising a sgRNA transcription initiation site;
(b) a polymerase promoter upstream from the sgRNA transcription initiation site, and
(c) one or more modified nucleotides at the 5′ end of the antisense strand of the dsDNA template.

Embodiment 18. The partially ssDNA template of embodiment 17, comprising a transcriptional enhancer sequence upstream of the polymerase promoter.

Embodiment 19. The partially ssDNA template of embodiment 17, wherein one or more modified nucleotide is 2′-O-methyl modified nucleotide.

Embodiment 20. The partially ssDNA template of embodiment 17, wherein single stranded DNA is complementary to all or a portion of the polymerase promoter.

Embodiment 21. The partially ssDNA template of embodiment 17, wherein the polymerase promoter is selected from the group consisting of T7 polymerase promoter, a T3 polymerase promoter, an SP6 polymerase promoter, a Syn5 polymerase promoter, and an E. coli RNase promoter.

Embodiment 22. A method of making a single guide ribonucleic acid (sgRNA) by in vitro transcription (IVT), comprising the steps of:

(a) obtaining a DNA template of any of embodiments 1-21, and
(b) making a sgRNA transcript by in vitro transcription.

Embodiment 23. The method of making sgRNA of embodiment 22, further comprising the step of:

(c) purifying the produced sgRNA transcript using qPCR.

Embodiment 24. The method of making sgRNA of embodiment 22, further comprising the step of:

(c) purifying the produced sgRNA transcript by reverse-phase chromatography.

Embodiment 25. The method of making sgRNA of any of embodiments 22-24, further comprising the step of:

(d) testing the purified produced sgRNA transcript for the presence of immune stimulating moieties by an immunogenicity assay.

Embodiment 26. A composition of single guide ribonucleic acid (sgRNA) transcripts, made by the process of any of embodiments 22-25, wherein:

(a) the composition of the sgRNA transcript is substantially free of immune stimulating moieties, and
(b) the composition is substantially free of sgRNA transcripts having n−1 mutations or n+1 mutations in the crRNA section of the sgRNA transcripts.

Embodiment 27. The composition of sgRNA transcripts of embodiment 26, wherein the sgRNA comprises pseudouridine (ψ), or 5-methylcytidine (m⁵C), or both and m⁵C.

Embodiment 28. The composition of sgRNA transcripts of embodiment 26, wherein the sgRNA transcripts in the composition are about 50 bases to 150 bases in length.

Embodiment 29. The composition of sgRNA transcripts of embodiment 26, wherein the sgRNA transcripts are dephosphorylated or capped at the 5′ end, at the 3′ end, or at the 5′ and 3′ ends.

Embodiment 30. A pharmaceutical composition, comprising the sgRNA transcripts of any of embodiments 26-29, in a pharmaceutically acceptable carrier.

### EXAMPLES

### Example 1

### Design of RNA-Encoding Polynucleotide Constructs, Including CRISPR Guide RNA Constructs

The process of design and synthesis of sgRNA can include design of an in vitro transcription (IVT) template, synthesis of designed sequence, insertion into appropriate vector to generate plasmid based template DNA, amplification of the plasmid, purification, linearization, purification of linearized template, IVT reaction to synthesize sgRNA and purification of sgRNA. Purified sgRNA may undergo additional enzymatic steps, such as phosphatase treatment, or capping, etc.

Design is an important first step that can originate with generating a DNA plasmid encoding several important features to generate RNA by in vitro transcription. See, FIG. 1. A T7 polymerase promoter, from which RNA is transcribed by the T7 RNA polymerase, can be placed upstream of the initiation site for the RNA. The RNA polymerase promoter can also be T7, T3, SP6, Syn5, E. coli or some other RNA polymerase known to those of skill in the biotechnological arts. Promoters can be supplemented by enhancer sequences upstream of RNA polymerase recognition site. The choice of RNA polymerase promoter used in the IVT cassette design mainly determines the transcription initiation nucleotide. If T7 RNA polymerase is used, sgRNA IVT synthesis will initiate either from G or A. One design of sgRNA sequences has been previously described by Jinek et al. (2012) Science 337:816-821. See also, Larson et al. (2013) Nature Protocols 8:2180-2196.

Another feature of some of the DNA templates described herein is a linearization site. One can include a linearization sequence in the design to ensure a specific 3′-end to the sgRNA. The linearization sequence can be a restriction endonuclease site precisely at the 3′ end of sgRNA sequences, e.g., a restriction endonuclease site with either blunt ends or a 5′ overhang. The linearization site can consists of a unique restriction enzyme site that, when cut, leaves a precise end for transcription to run off. After the sgRNA sequence, a restriction site can be included for linearization (e.g. DraI, BspQI, SapI, BbsI, etc.). The template can be screened for the presence of selected enzyme recognition sites, to ensure that site is uniquely locating at 3′-end of sgRNA sequences.

An alternative to using linearization site is a ribozyme sequence for the formation of precise 3′ or 5′ ends of sgRNA during the IVT reaction. Ribozymes are self-cleaving RNA sequences that are inserted after the end of the RNA sequence. Upon transcription, the ribozyme sequence will cleave off, leaving a precise end to the RNA. In some embodiments, the DNA template can include a linearization site downstream of a ribozyme sequence to allow for linearization of a DNA plasmid for IVT. Ribozymes are self-cleaving RNA sequences that allow for the formation of precise 3′ or 5′ end of sgRNA after completion of IVT reaction. RNA polymerase termination sequences can also be used to provide precise 3′ end to the sgRNA transcript. In some embodiments, when the DNA template includes an RNA polymerase termination sequence, the DNA template can also include a linearization sequence, e.g., downstream of the termination sequence to allow for linearization of a DNA plasmid for IVT.

The design of a template for in vitro transcription can be plasmid-based for amplification in Escherichia coli, or a dsDNA oligonucleotide, or a partially ssDNA oligonucleotide. The dsDNA portion of a partially ssDNA oligonucleotide structure can include, e.g., all or a portion of the sgRNA sequence.

The process of design and synthesis of sgRNA can include the design of the template, synthesis of designed sequence, insertion into appropriate vector to generate plasmid based template DNA, amplification of it, purification, linearization, purification of linearized template, IVT reaction to synthesize sgRNA, purification of sgRNA. Purified sgRNA may undergo additional enzymatic manipulations, such as phosphatase treatment, or capping.

The DNA template can be inserted into an appropriate vector plasmid DNA capable to amplify in Escherichia coli or another host, using techniques such as ligation, TA cloning, In-Fusion, etc. See, Molecular cloning: A laboratory manual. Second edition. Volumes 1, 2, and 3. Current protocols in molecular biology. Volumes 1 and 2. (Cold Spring Harbor Press); Green & Sambrook Molecular Cloning: A Laboratory Manual. (Cold Spring Harbor Laboratory Press, 2012).

Alternatively, a DNA template synthesized by chemical methods can be used. Moreover, a DNA template can be generated by PCR amplification of the template. See, Molecular cloning: A laboratory manual. Second edition. Volumes 1, 2, and 3. Current protocols in molecular biology. Volumes 1 and 2. (Cold Spring Harbor Press); Green & Sambrook Molecular Cloning: A Laboratory Manual. (Cold Spring Harbor Laboratory Press, 2012). Methods of PCR generation of DNA templates are shown in FIG. 4 and FIG. 5.

The DNA template can include chemically modified DNA template sequences produced by chemical solid-phase synthesis. A general production procedure is provided by Beaucage et al. (1981) Tetrahedron Lett. 22, 1859-62, and by McBride & Caruthers (1983) Tetrahedron Lett. 24, 245-8.

### Example 2

LC-MS Analyses of sgRNAs Produced by In Vitro Transcription

LC-MS analyses of 100 mer and 110 mer CRISPR sgRNAs produced by IVT using T7 RNA polymerase showed that one or two additional non-template nucleotides were being added to the 3′ end of the sequence during IVT. Non-templated addition of nucleotides by T7 polymerase has previously been reported by Nacheva & Berzal-Herranz (2003) Eur. J. Biochem. 270, 1458-1465. Non-template addition of nucleotides prevents the IVT production of RNA with precisely defined 3′ ends,

T7 polymerase and other RNA polymerases can transcribe RNA using single stranded DNA templates as well as RNA and RNA:DNA chimera templates. See, Milligan et al. (1987) Nucleic Acids Res. 15, 8783-8798 and Arnaud-Barbe et al. (1998) Nucleic Acids Res. 26, 3550-3554.

Synthetic single and/or double stranded DNA or RNA that have steric or un-natural tags on the end of the sequence can help “kick-off” the RNA polymerase and prevent unwanted non-template extension. Kao et al. (1999), RNA, 5: 1268-1272 has described using modified DNA templates to eliminate n+1 additions to the 3′ end of in vitro transcribed RNA. No such approach has been applied to generate an IVT-made sgRNA or gRNA prior to the instant study.

To test this hypothesis, template DNA with the same modifications made by Kao et al., as well as a biotin modification, were ordered from Integrated DNA Technologies (IDT) (Coralville, Iowa, USA). All DNA templates were polyacrylamide gel electrophoresis (PAGE) purified.

TABLE 1

| Oligo | Oligo | |
|---|---|---|
| # | nickname | DNA sequence 5′-3′ |
| 11 | template | AAAAGCACCGACTCGGTGCCACTTTTTCAAGTTGAT |
| | | AACGGACTAGCCTTATTTTAACTTGCTATTTCTAGC |
| | | TCTAAAACtgaagaagatggtgcgctccTATAGTGA |
| | | GTCGTATTAcaattctccggcctccggatcc |
| | | (SEQ ID NO: 11) |
| 12 | template | /5-bio/ |
| | biotin | AAAAGCACCGACTCGGTGCCACTTTTTCAAGTTGAT |
| | | AACGGACTAGCCTTATTTTAACTTGCTATTTCTAGC |
| | | TCTAAAACtgaagaagatggtgcgctccTATAGTGA |
| | | GTCGTATTAcaattctccggcctccggatcc |
| | | (SEQ ID NO: 12) |
| 13 | non- | GGATCCGGAGGCCGGAGAATTGTAATACGACTCACT |
| | template | ATAGGAGCGCACCATCTTCTTCAGTTTTAGAGCTAG |
| | | AAATAGCAAGTTAAAATAAGGCTAGTCCGTTATCAA |
| | | CTTGAAAAAGTGGCACCGAGTCGGTGCTTTT |
| | | (SEQ ID NO: 13) |
| 14 | non- | GGATCCGGAGGCCGGAGAATTGTAATACGACTCACT |
| | template | ATAGGAGCGCACCATCTTCTTCAGTTTTAGAGCTAG |
| | minus 4T | AAATAGCAAGTTAAAATAAGGCTAGTCCGTTATCAA |
| | | CTTGAAAAAGTGGCACCGAGTCGGTGC |
| | | (SEQ ID NO: 14) |
| 15 | template | mAmAAAGCACCGACTCGGTGCCACTTTTTCAAGTTG |
| | 2′Ome | ATAACGGACTAGCCTTATTTTAACTTGCTATTTCTA |
| | | GCTCTAAAACtgaagaagatggtgcgctccTATAGT |
| | | GAGTCGTATTAcaattctccggcctccggatcc |
| | | (SEQ ID NO: 15) |

The DNA template was brought up in deionized water, annealed at 95° C. for 5 min and cooled on a laboratory bench top to room temperature. The IVT product was LiCl-purified before LC-MS analysis.

In-vitro transcription requires a linear DNA template containing a promoter, ribonucleotide triphosphates, a buffer system that includes DTT and magnesium ions, and a T7 RNA polymerase. In some embodiments, the linear DNA template is purified.

For LC-MS analysis, samples were analyzed on a Thermo Q-EXactive instrument with a Waters Acuity BEH 2.1×100 mm HPLC column held at 75° C. The mobile phase A was 200 mM hexafluoroisopropanol/8.15 mM triethylamine/0.75 μM EDTA, pH=8. The mobile phase B was methanol. The initial flow rate was 300 μL/min. The gradient conditions were started at 5% mobile phase B, then 13% mobile B at 0.6 min, followed by linear ramping to 21% at 14 min, 90% at 18 min and then returning to 5% at 18.5 min.

The MS was operated in negative ion mode scanning from 700-2800 m/z.

An 87-mer RNA standard was run to check LC-MS performance. Samples were at 10 μg/mL and 10 μL was injected onto the column.

The results of the deconvoluted mass spectra showed that a CRISPR RNA of exact length can be generated using IVT conditions. The biotin addition did not produce as great a reduction in n+1 as did using two 2-O-methyl modifications on the end of the template, which when combined with a slightly shorter non-template strand completely eliminated the 3′ n+1.

TABLE 2
SYNTHETIC DNA TEMPLATES

| | |
|---|---|
| Oligos 11 + 13 of Table 1 | template + nontemplate |
| Oligos 12 + 13 of Table 1 | template biotin + nontemplate |
| Oligos 11 + 14 of Table 1 | template + nontemplate − 4T |
| Oligos 12 + 14 of Table 1 | template biotin + nontemplate − 4 T |
| Oligos 15 + 13 of Table 1 | 2′O-methyl template + nontemplate |
| Oligos 15 + 14 of Table 1 | 2′O-methyltemplate + nontemplate − 4T |

### Results

Synthetic templates allow IVT RNA synthesis, but result in n+1 IVT products.

Biotin addition reduces n+1. A shorter non-template also helps reduce n+1.

2′ O-methyl template greatly reduces n+1. Addition of shorter non-template eliminates n+1.

Normal IVT from PCR template shows significant n+1 product.

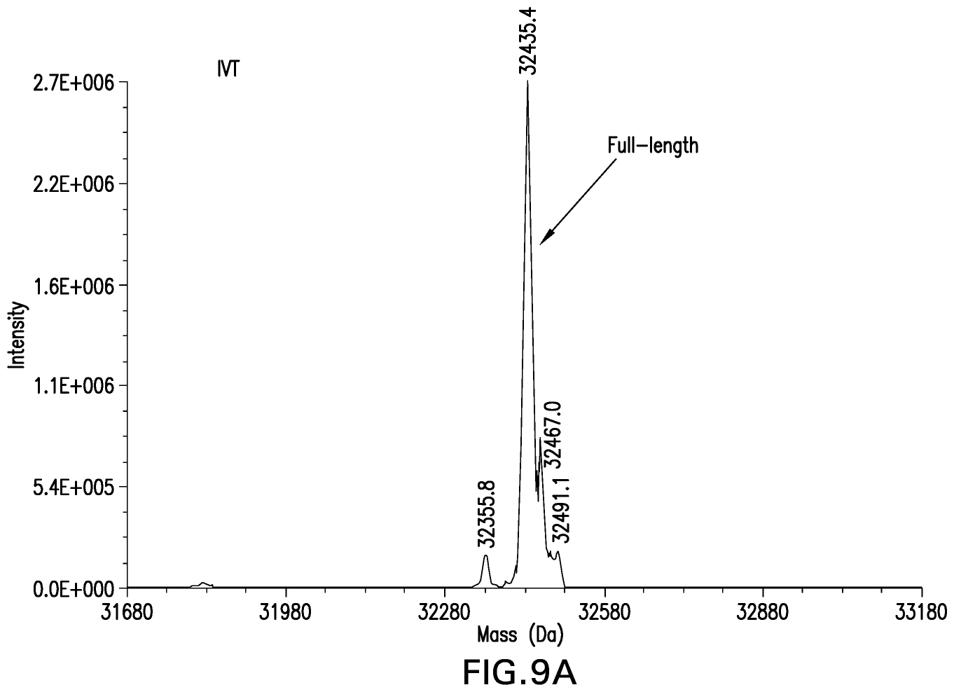

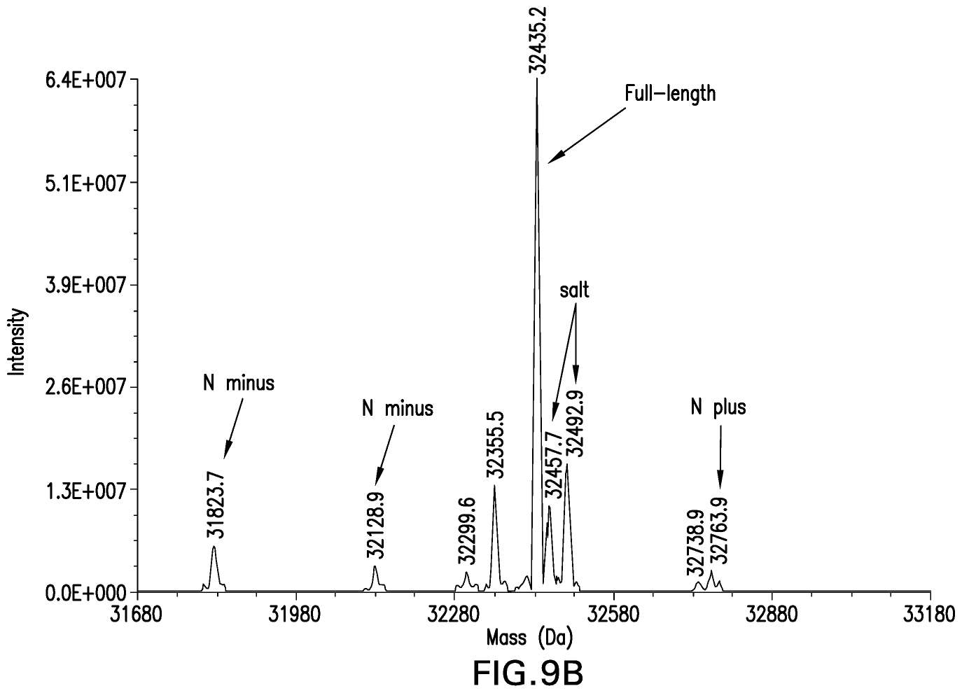

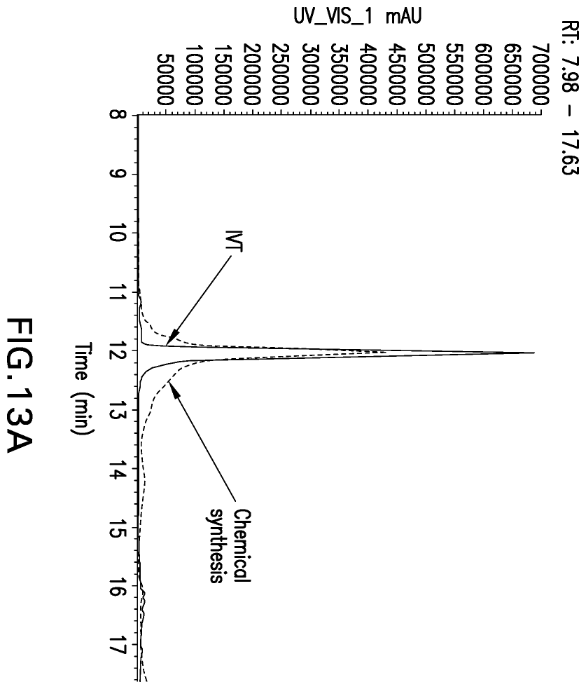

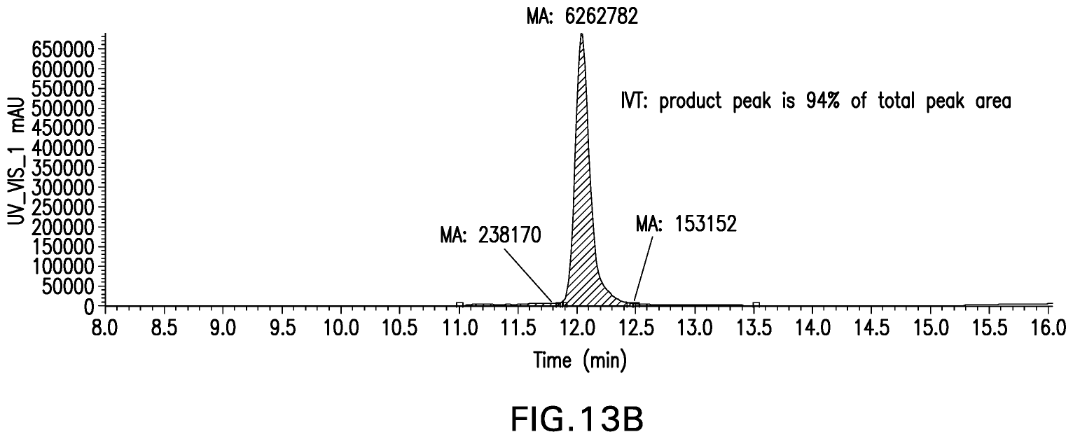

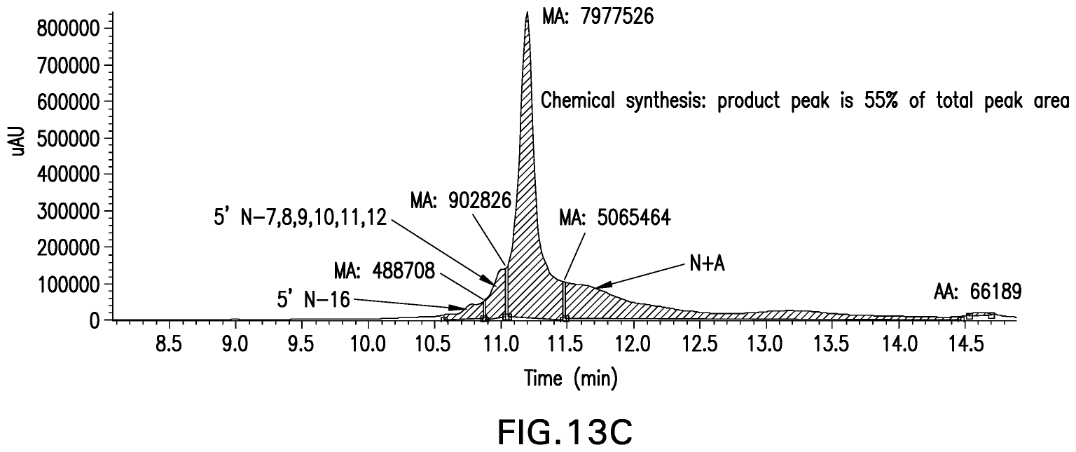

LC-MS was used in this study to show the specific product species in the final product (e.g., the expected full length product, the n+x variants, the n−x variants, the salts, etc.) (see, e.g., FIGS. 9A and 9B). Chromatograms UV260 nm, on the other hand, was used in this study to show the purity of the final product (e.g., FIGS. 13A-13C).

As shown in FIG. 9A, it is possible to eliminate the n+1 products although some sequences can still produce minor amounts of n+1 and n−1 as shown in FIG. 9B.

FIG. 9B is the mass spectra of the entire chromatographic peak for the IVT produced mRNA shown in FIG. 13A. The relative impurities still result in a purer final product compared to the chemically synthesized material as shown in TABLE 3 below and by the narrower chromatographic peak in FIG. 13A and FIG. 13B. Importantly, due to the action of the enzymes involved in IVT, the site of the x additions are known to be located at the 3′ end. The 3′ end of the sgRNA, however, is less critical than its 5′ end in CRISPR-editing.

TABLE 3

| % Total | Identity |
|---|---|
| 84 | Full-length product |
| 5 | −UU |
| 3 | −U |
| 3 | +G |
| 3 | +C |

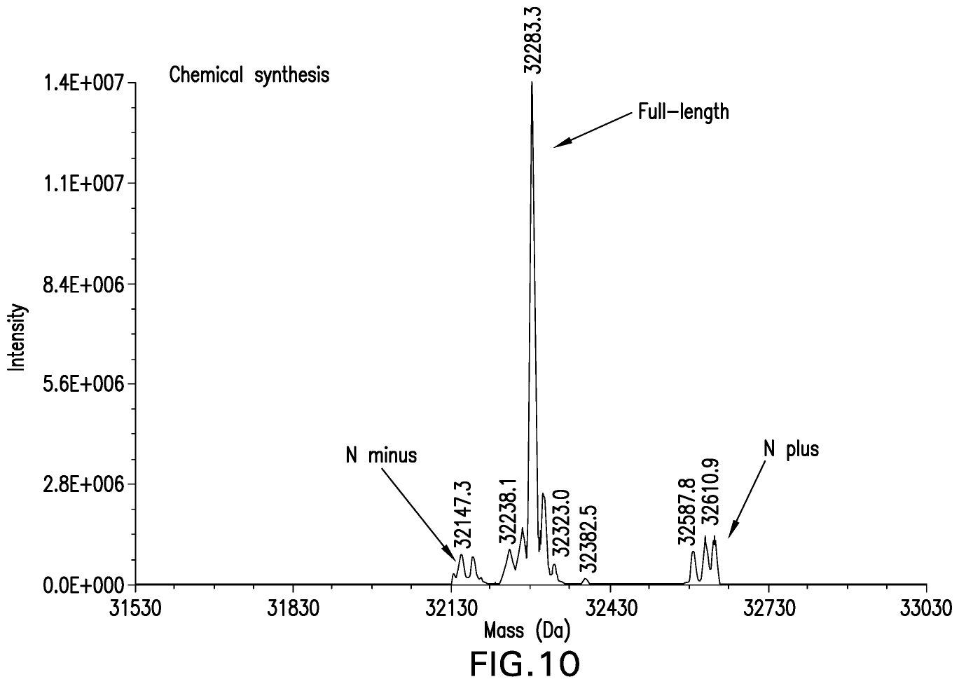

By contrast, as shown in FIG. 10, a mass spectrum of a heart cut or center of the chemical synthesis chromatographic peak in FIG. 13A shows similar n+x are also formed during chemical synthesis of sgRNA. See TABLE 4 below. The broad chromatographic peaks in FIGS. 13A and 13C contain many n+ and n− species in the leading and tailing regions of the peak not present in the heart cut. Due to the nature of chemical synthesis, the insertions (leading to n+x variants) and/or the deletions (leading to n−x variants) are located randomly throughout the sequence.

TABLE 4

| % Total | Identity |
|---|---|
| 75 | Full-length product |
| 6 | +G |
| 5 | +A |
| 4 | +C/U |
| 3 | depurination |
| 3 | depyrimidation |
| 2 | acetyl protecting group |

Therefore, the IVT-made RNA (e.g., sgRNA) had more predicable n+x or n−x variants than those of chemically synthesized RNA. More importantly, the IVT-made RNA (e.g., sgRNA) had much higher purity than the purity of the chemically synthesized RNA, see, FIGS. 13A-13C.

### Example 3

### Plasmid Amplification, Isolation and Linearization and Purification of the DNA Template as the Basis for In Vitro Transcription

### Materials

Competent E. coli cells (New England Biolabs, part #C3019H)

Eppendorf tubes

Nuclease free water (Ambion, Cat No. AM9937).

Heat block and water bath.

Oven at 37° C.

SOC media (Life Technologies, part #15544-034; 2% tryptone, 0.5% yeast extract, 10 mM NaCl, 2.5 mM KCl, 10 mM MgCl₂, 10 mM MgSO₄, and 20 mM glucose).

Agar plate.

Qiagen Plasmid Maxi Kit, Qiagen part #12163.

Sigma-Aldrich ethyl alcohol, Pure Cat No. 459844.

Sigma-Aldrich isopropanol (Cat no. 19516-500 mL) molecular biology grade.

ThermoFisher Nanodrop 8000 spectrophotometer.

NEB restriction enzyme, BSPQ1, Cat no. R0712L, 2,500 units, 10,000 units/mL.

NEB 10× NEBuffer 3.1.

Lonza agarose, Cat No. 50004.

BioRad ethidium bromide solution, Cat No. 161-0433.

Invitrogen high molecular weight ladder.

10×TAE buffer.

Qiagen 2500 tip, Cat No. 10083.

### Plasmid Amplification

Competent E. coli cells (New England Biolabs, part #C3019H) are thawed on ice for 10 min. These are pre-aliquoted as 50 μL per tube.

A volume of 0.1-10 ng (dissolved in 1-5 μL of water) of plasmid DNA is added to each aliquot of cells.

Flick the tubes 4-5 times to mix cells and DNA. Do NOT vortex the cells.

Incubate on ice for 5 min.

The tubes are heat-shocked in the 42° C. water bath for exactly 30 sec followed by incubation on ice for 5 min.

Add 900 μL of preheated (42° C.) SOC media (Life Technologies, part #15544-034; 2% tryptone, 0.5% yeast extract, 10 mM NaCl, 2.5 mM KCl, 10 mM MgCl2, 10 mM MgSO4, and 20 mM glucose).

Incubate at 37° C. for 1 hr with shaking at 225-250 rpm.

Add 50 μL of SOC media to the center of a 37° C. agar plate.

20 μl of the transformation mixture is pipetted on the center of each agar plate.

Spread the transformation mixture on the plates using plating beads or cell spreader.

Incubate plates at 37° C. overnight.

### Isolation of DNA Plasmid (Using Qiacen Plasmid Maxi Kit, Qiacen Part #12163)

Pick a single colony of cells and inoculate in 100 ml LB medium broth (Life Technologies, part #10855-001) for high-copy plasmids or 500 ml for low-copy plasmids. Add 1 mL of 100 mg/mL of antibiotic to 1 L of LB broth.

Grow the culture in flask with a volume of at least 4 times the volume of the culture. Incubate the culture flask in an incubator overnight at 37° C. with shaking (˜200 rpm).

The cells are harvested by filling conical centrifugation bottles and centrifuged at 6000×g for 30 min at 4 C. Pour off the supernatant.

Follow Qiagen Plasmid Maxi Kit directions to isolate plasmid DNA:

A volume of 10 ml of Qiagen Buffer P1 (from Qiagen Maxi Kit, with RNase added) is added to the pellet of cells for resuspension.

The pellet may be vortex mixed in the P1 buffer in order to completely break up the pellet.

Add 10 mL Buffer P2 (from Qiagen Maxi Kit) is added and mixed by inverting 4-6 times. This mixture is incubated at room temperature for 5 min.

Add 10 mL of chilled Buffer P3 (from Qiagen Maxi Kit) is added and mixed by inverting 4-6 times then incubated on ice for 20 min. The contents of each bottle are poured into 50 ml centrifugation tubes suitable for centrifugation speeds >20,000×g. The tubes are centrifuged at >20,000×g for 30 min at 4 C.

The supernatant containing plasmid DNA is transferred into a separate containers and kept on ice. A QIAGEN-tip 500 (from Qiagen Maxi Kit) is equilibrated by applying 10 ml Buffer QBT (from Qiagen Maxi Kit). The column is emptied by gravity flow. The supernatant containing the DNA is poured onto the QIAGEN-tip and enters the resin by gravity flow. The QIAGEN-tip is washed with two volumes (2×30 ml) of Buffer QC (from Qiagen Maxi Kit).

Elute DNA with 15 ml Buffer QF (from Qiagen Maxi Kit) and the eluate is collected in a 30 ml tube suitable for centrifugation speeds >20,000×g.

Precipitate DNA by adding 10.5 ml (0.7 volumes) of room-temperature isopropanol to the eluted DNA. The pellet is mixed and centrifuged at >15,000×g for 30 min at 4 C. The supernatant is discarded.

Wash plasmid DNA with 5 ml of room-temperature 70% ethanol, and centrifuged at >15,000×g for 10 min. The supernatant is discarded without disturbing the pellet. The pellet of DNA is air dried for 5-10 min then dissolved in nuclease free water.

Obtain concentration on ThermoFisher Nanodrop 8000 spectrophotometer (ng/μL)

### Linearization

Set up digestion as follows in a small flask. Add in the order listed.

TABLE 5
LINEARIZATION REACTION SOLUTION

| Total volume: 40,000 μL = 40 mL | volume (μL) |
|---|---|
| sterile filtered milli-Q-water | 1332 |
| 10× NEBuffer 3.1 (1×) | 400 |
| Circular DNA (967 ng/μL) = 9670 μg | 2068 |
| BspQ1 (1 unit/1 μg DNA) | 200 |
| Total 40 mL reaction | |

The digest is incubated for 2 hrs at 50° C. Digestion in small flask, twirling in a water bath at 50° C. @ 80 rpm.

Check for complete linearization by running 0.8% agarose gel to separate forms of DNA. For comparison, see, FIG. 3.

### Preparation for 0.8% Agarose Gel:

1×TAE buffer: 20 mL 50×TAE buffer+980 mL milli-Q-water.

0.8% agarose gel: (SeaKem LE agarose Lonza cat #50004, lot Ag501L).

2.4 g agarose in 300 mL of 1×TAE buffer+10 μL Bio-Rad ethidium bromide 10 mg/mL, cat #161-0433).

Linearized plasmid migrates between the nicked and the supercoiled forms of DNA. Note: The gel is overloaded to be able to detect any circular or nicked form of DNA that is present.

After complete linearization the digest is cleaned by using Qiagen 2500 tip.

Equilibrate Qiagen-tip 2500 with 30 mL QBT buffer.

Mix 25 mL QBT buffer with digest. Apply to tip. Allow to gravity flow.

Wash 3×30 mL QC buffer.

Elute with 30 mL QF buffer warm the buffer at 37° C. for higher recovery.

Precipitate DNA using 22 mL 2-propanol, spin 15,000×g for 30 min.

Wash pellet with 3×70% ethanol.

Reconstitute using nuclease-free water.

Read OD by Nanodrop spectophotometer.

Store linearized DNA at −20° C. until use.

### Example 4

In Vitro Synthesis of sgRNA

### Materials for In Vitro Transcription Reaction:

Linear plasmid DNA (1 μg/μL).

1M Tris-HCl pH 8.0 (Sigma).

1M magnesium chloride (Sigma, Cat No. M1028-100 mL).

ATP, 100 mM (New England Biolabs, Cat No. N0451B).

5′-methyl CTP, 100 mM (Trilink, Cat No. N-1014).

GTP 100 mM (New England Biolabs, Cat No. N0452B).

Pseudo UTP 100 mM (Trilink, Cat No. N-1019).

DTT 1M (Sigma, Cat No. 43816).

Spermidine 100 mM (Sigma, S0266-16).

Pyrophosphatase 0.1 U/μl (New England Biolabs, Cat No. M2403B).

RNase Inhibitor 40 U/μl (New England Biolabs, Cat No. M0307B).

T7 RNA polymerase 50 U/μl (New England Biolabs, Cat No. M0251B).

Nuclease free water (Ambion, Cat No. AM9937).

LiCl 7.5 M (Ambion, Cat No. AM9480).

Ethyl alcohol, pure (Sigma-Aldrich, Cat No. 459844).

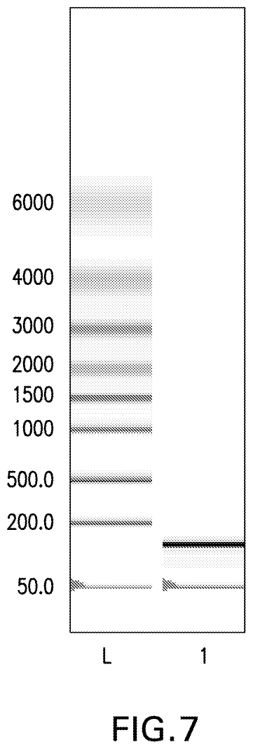

See, FIG. 7

TABLE 6
IN VITRO TRANSCRIPTION PROTOCOL

| Step | |
|---|---|
| 1 | Set up 100 μL reaction volume (1×) by adding the following |
| | components in this order: |
| | 4 μL of Tris-HCl pH 8 (1M Sigma), |
| | 2.4 μL MgCl₂ (1M Sigma), |
| | 6 μL of each NTP (100 mM adenosine-5′-triphosphate, cytosine- |
| | 5′-triphosphate, guanosine-5′-triphosphate, uridine-5′- |
| | triphosphate), |
| | 1 μL of DL-dithiothreitol (DTT), |
| | 2 μL spermidine (100 mM, Sigma), |
| | 20 μg of linearized DNA, |
| | 2 μL Pyrophosphatase 0.1 U/μL, New England Biolabs), |
| | 2.5 μL RNase inhibitor (40 U/μL, New England Biolabs), |
| | 10 μL T7 RNA Polymerase (50 U/μL, New England Biolabs). |
| 2 | Incubate reaction at 37° C. for 5 hours. |
| 3 | Precipitate sgRNA is by adding 75 μL of 7.5M LiCl (Ambion) |
| | (2.81M final concentration), store at −20° C. |
| 4 | Centrifuge sample at 15,000 g for 30 min, remove supernatant |
| | completely, avoiding to dislodging the pellet. |
| 5 | Add 500 μL of 70% ethanol. Gently mix, repeat step 4. |
| 6 | Repeat step 5. |
| 7 | Remove all of the liquid and air-dry pellet for 15 min. |
| 8 | Reconstitute using nuclease free water so the final concentration |
| | is 2000 ng/μL or below. |

### Modification of 5′End of the Transcript

RNA that is produced by IVT contains a triphosphate moiety at its 5′ end. In order to reduce a potential undesired interferon response triggered by 5′triphosphate RNA, the RNA should ideally be dephosphorylated according to protocol below. The amounts can be scaled up depending on the amounts of sgRNA needed to be dephosphorylated.

TABLE 7
PROTOCOL FOR DEPHOSPHORYLATION OF 5′-ENDS OF sgRNA
SYNTHESIZED BY IN VITRO TRANSCRIPTION

| | | | |
|---|---|---|---|
| 1 | Prepare reaction as follows: | | |
| | IVT synthesized sgRNA | 1 | mg |
| | 10× CutSmart Buffer | 7.5 | ml |
| | CIP (10 U/μl) NEB; M0290L | 6500 | units |
| | RNAse inhibitor (40 U/ul) | 1875 | ul |

| | |
|---|---|
| RNAse free H2O | Up to 75 mL |

| | |
|---|---|
| 2 | Incubate at 37 C. with gentle shaking for 2 hr, then stored at |
| | −20° C. until RP-HPLC purification |

RNA transcript also can be capped to have Cap-0, or Cap-1 on it's 5′ end to remove 5′ triphosphates. The amounts can be scaled up depending on the amounts of sgRNA needed to be capped.

TABLE 8
Cap-0 capping reaction

| 1 | Prepare reaction as follows: | | |
|---|---|---|---|
| | RNA | 10 | ug |
| | RNAse free H2O | Bring up to | |
| | | 14.5 ul volume | |
| | Incubate at 70 C. for 2 min, then ice for 2 mins | | |
| | Add to the denatured RNA: | | |
| 2 | 10× Capping Buffer | 2 | ul |
| | GTP (10 mM) | 1 | ul |
| | SAM (2 mM, dilute 32 mM stock) | 1 | ul |
| | RNase Inhibitor (40 U/ul) | 0.5 | ul |
| | Vaccinia Capping Enzyme (10 U/ul) | 1 | ul |

| | |
|---|---|
| 3 | Incubate at 37 C. for 1 hr, then stored at −20° C. until RP-HPLC |
| | purification |

TABLE 9
Cap-1 capping reaction

| 1 | Prepare reaction as follows: | | |
|---|---|---|---|
| | RNA | 10 | ug |
| | RNAse free H2O | Bring up to | |
| | | 14.5 ul volume | |
| | Incubate at 70 C. for 2 min, then ice for 2 mins | | |
| | Add to the denatured RNA: | | |
| 2 | 10× Capping Buffer | 2 | ul |
| | GTP (10 mM ) | 1 | ul |
| | SAM (2 mM, dilute 32 mM stock) | 1 | ul |
| | RNase Inhibitor (40 U/ul) | 0.5 | ul |
| | Vaccinia Capping Enzyme (10 U/ul) | 1 | ul |
| | mRNA Cap 2′-O-Methyl Transferase, (50 U/ul) | 1 | ul |

| | |
|---|---|
| 3 | Incubate at 37 C. for 1 hr, then stored at −20° C. until RP-HPLC |
| | purification |

Alternatively, 5-capped RNA can be produced using ARCA capping reagents.

### Example 5

### Purification of RNA Synthesized by In Vitro Transcription by Reverse Phase Chromatography

RNA is produced using in vitro transcription. To remove contamination from in vitro transcribed RNA HPLC purification method is needed. This method is scalable and can be easily performed by one of skill in the biotechnological art. HPLC reverse phase purification has shown to remove immune stimulation species and full length DNA.

HPLC purification materials. Use RNase-free and HPLC grade reagents, whenever possible. Acetonitrile is toxic, so ensure proper protection is used. A HPLC system that can monitor the presence of material at 260 nm and that is fitted with a fraction collector. This method uses an AKTA Explorer FPLC instrument with:

P-900 flow controller.

UV-900 UV detector collecting at 260 nm, 280 nm, and 230 nm.

pH/C-900 conductivity and pH detector.

Frac-950 fraction collector.

Unicorn Processing Software.

TL105 column heater (Timberline Instruments, Boulder, Colo.).

HPLC column: Phenomenex Luna C18(2) (00D-4252-U0-AX).

### Buffer A: 0.1 M Triethylammonium Acetate (TEAA), pH 7.0 (Part Number: 90357) (Fluka).

Add 200 ml TEAA. Add 1800 ml di-water.

### Buffer B: 0.1 M TEAA, 50% Acetonitrile, pH 7.0 (Part Number: 90357) (Fluka) & Part Number: BDH83639) (BDH)

Add 200 ml TEAA. Add 900 ml acetonitrile and 900 ml di-water.

Acetonitrile: 50% for column storing.

Acetic acid: 3% for column and HPLC system cleaning.

Use HPLC grade water.

Ethanol: 20% for long-term storage of HPLC system.

### Tangential Flow Filtration and Diafiltration Desalting Methods

RNA purification can be done after the RNA is synthesized through in vitro transcription, or after the RNA is capped using a Vaccina capping reaction. The sample is normally cleaned up using a LiCl precipitation reaction to remove excess free nucleotides and other enzymes. The process can be scaled up or scaled down by matching column volumes.

Vivaspin 20 spin columns (30,000 MWCO) (GE Healthcare) (part number: 28932361).

### Reverse Phase Purification of 50 mg RNA on a 50 mL Column

Set column oven to 65° C.

Dilute RNA 1:1 with 9% Buffer B before injecting to the column.

Set the flow rate to 50 ml/min. Equilibrate column with 8 column void volumes of 9% buffer B.

Load RNA onto column at 5 ml/min using S1 inlet.

Wash the column with 3 column void volumes of 9% buffer B.

Run a linear gradient from 0% to 9% buffer B over 5 column void volumes.

Run a linear gradient from 9% to 35% buffer B over 27 column void volumes.

Collect 10 ml fractions (Specifications: UV₂₆₀>100 mAU).

TABLE 10
REVERSE PURIFICATION GRADIENT

| CV | % B | Flowrate |
|---|---|---|
| 5 CV | 9 | 50 mL/min |
| 27 CV | 35 | 50 mL/min |
| 5 CV | 58 | 50 mL/min |
| 5 CV | 9 | 50 mL/min |

Equilibrate with 9% Buffer B for 5 column void volumes.

Nanodrop quantitation of RNA in fractions: Fractions were tested for the presence of RNA using a Nanodrop UV reader at 260 nm using the standard RNA setting (default correlation of 1 abs unit=40 ng/μL).

DNA concentration of each fraction is then translated to total amount of RNA by multiplying the concentration by the fraction volume. Example: Fraction concentration=10 ng/μL. Fraction volume=14 mL. Fraction RNA amount=140 μg RNA (14*10).

The total amount of material across all fractions is calculated by adding the total amount of RNA in each fraction. This can be used to determine the chromatography yield by dividing this amount by the total amount of material that was loaded onto the column.

### Fraction Desalting

After determining the concentrations of each fraction, a subset of these fractions will be chosen for further analysis. Since RNA long term stability in acetonitrile is unknown, selected fractions needed to be desalted immediately.

These fractions are desalted using Vivaspin 20 spin columns from GE Healthcare (30,000 MWCO).

1-15 mL (˜1 mg) of each test fraction is added to well-labeled spin columns. Split a fraction into multiple spin columns if necessary.

Filters are spun at 4400 g for 8 min and RT in a fix angle rotor in a bench-top centrifuge.

Flow-through is discarded, or the skilled artisan can test for UV260 nm on Nanodrop to ensure no RNA leaks through.

Filters are then washed with 15 mL dlH₂O.

Filters are spun at 4400 g for 10 min and RT in a fix angle rotor in a bench-top centrifuge.

Flow-through is discarded.

dlH₂O wash is repeated as above.

Third wash was with 15 mL dlH₂O.

Filters are spun at 4400 g for 10 min and RT in a fix angle rotor in a bench-top centrifuge. Volume in each spin filter should be ˜50-250 μL.

Collect each fraction. If one fraction was desalted in multiple spin column, pooled all together. Transfer each fraction into 2 ml deep well plate.

Samples are tested for concentration and spectral purity (260/280 and 260/230) on a Nanodrop instrument as before.

### Fraction Analytics

The desalted fractions are diluted to ˜65 ng/μL with dlH₂O (exact concentrations measured) and placed in a 96-well plate with appropriate controls. Samples are submitted for size-exclusion chromatography UPLC assay for purity analysis. These analytics help inform as to how to pool the fractions for final desalting. The final fraction cutoffs are as follows: RNA purity should be >70% or >70% of pre-purification purity.

qPCR assay for residual DNA plasmid. RNA Fraction should have <30 μg DNA/μg of RNA.

qPCR assay for negative strand quantitation. RNA Fraction should have <5% negative strand compared to total RNA.

THP-1 monocytic cellular immunogenicity assay. The RNA fraction should have SEAP levels that are similar to previously purified samples and lower than the pre-purification control.

The fractions that fit these criterions are pooled.

### Final Sample Analysis

Final sample concentration is measured on the Nanodrop instrument for yield and UV260/280 nm and UV260/230 nm purity. Endotoxin is measured on the final sample.

The final sample undergoes all of the same analytics listed in the fraction analytics section supra.

### Example 6

IVT of SGRNA Beginning with a Nucleotides

### Materials and Methods

### DNA Linearization

Plasmid DNA is linearized with restriction enzyme to generate linear DNA template for use in the in vitro transcription reaction (see Table 5).

Run 1 ul of the digest reaction on a 1% agarose gel at 95V for 1 hr to check the linearized product. If DNA appears to be well linearized continue to cleanup of DNA Linearization. If a substantial amount of circular DNA is remaining, 10 ul of restriction enzyme can be added to the reaction and incubated for an additional hour at 37 C.

### Cleanup of DNA Linearization

Purify the DNA digest using Qiagen columns, use appropriate column for amount of DNA digested.

For a standard digest of 500 ug of DNA, use the QIAGEN-tip 500 column to purify.
Equilibrate QIAGEN-tip 500 with 10 ml Buffer QBT
Mix the digest reaction with 10× volume of buffer QBT add apply to column.
Wash the column with 2×60 ml of Buffer QC
Elute DNA with 15 mL Buffer QF into a 50 ml tube and add 10.5 ml isopropanol. Mix and let sit for 1 h at −20 C.
Centrifuge at max speed for 1 h, wash DNA pellet with 5 ml of 70% ethanol, air dry the DNA pellet and resuspend the DNA in nuclease free water.

### In Vitro Transcription

The in vitro transcription reaction can be scaled up linearly for larger batches of RNA. The amount of template DNA added is dependent on the method used to generate linear DNA. If restriction digest was used to linearize plasmid DNA 10 ug of template per 1× reaction must be used. If the linear DNA was generated by PCR 2.5 ug of template pre 1× reaction is sufficient.

| | 1× reaction |
|---|---|
| 1M Tris-HCl pH 8.0 (Sigma T2694) | 4 |
| 1M MgCl (Sigma, M1028) | 2.4 |
| GTP 100 mM (NEB, N0452B) | 6 |
| CTP 100 mM (NEB, N0450B | 6 |
| ATP 100 mM (NEB, N0451B) | 6 |
| PseudoUTP 100 mM (Trilink, N1019) | 6 |
| DTT 1M (Sigma, 43816) | 1 |
| Sperimidine 100 mM (Sigma, S0266) | 2 |
| Linear DNA Template | 10 ug (template produced by |
| | restriction digest) or 2.5 ug |
| | (template produced by PCR) |
| Pyrophosphatase 0.1 U/ul | 2 |
| (NEB, M2403B) | |
| RNase Inhibitor 40 U/ul | 2.5 |
| (NEB, M0307B) | |
| T7 RNA Polymerase 50 U/ul | 10 |
| (NEB, M0251B) | |
| Nuclease free wáter | Up to 100 ul |
| Mix and Incubate at 37° C. for 2 hrs | |
| DNase 2 U/ul (NEB | 0.5 |
| Mix and Incubate at 37° C. for 30 min | |
| LiCl 7.5M (Ambion, AM9480) | 75 |

Mix and Incubate at −20° C. for 1 h

After incubation at −20° C. in LiCl, centrifuge RNA for 10 minutes to pellet the RNA. Remove supernatant and wash the RNA pellet with 500 ul of 70% ethanol and centrifuge again for 10 minutes. Remove ethanol, let pellet air dry for 5 minutes and resuspend the RNA in nuclease free water. The expected yield from a 1× reaction is approximately 250 ug for G initiated sgRNA template. 5′RACE

5′RACE system by Invitrogen (cat no. 18374-041) was used to perform the 5′RACE. First Strand cDNA synthesis was performed using 5′RACE primers and their respective RNA and SuperScript I reverse transcriptase. 5′RACE Primers:

| | |
|---|---|
| 5′RACE sgRNA2 | agcgcccaatacgcaaaccgcc |
| | (SEQ ID NO: 31) |

| Combine into PCR Tube: | |
|---|---|
| sgRNA2 concentration | 1.624 |
| (ug) | |
| 5′ RACE Primer (5 uM) | 0.5 |
| sgRNA (1-5 ug) | 1.00 |
| H2O | 14.00 |
| Final Volume | 15.5 |
| Incubate for 10 min at 70 C., then chill on ice 1 min. | |
| Add: | |
| 10× PCR Buffer | 2.5 |
| 25 mM MgCl₂ | 2.5 |
| 10 mM dNTP mix | 1 |
| 0.1M DTT | 2.5 |
| Final Volume | 8.5 |

Mix gently, centrifuge and Incubate for 1 min at 42 C.

Add 1 ul of SuperScript II RT. Mix and incubate for 50 min at 42 C.

Incubate at 70 C for 15 min to terminate the reaction.

Centrifuge briefly and place reaction at 37 C.

Add 1 ul of Rnase mix and incubate for 30 min at 37 C.

Place on ice or store at −20 C.

The cDNA was purified via SNAP Column purification:
1. Bring binding solution to RT and equilibrate 100 ul of H2O at 65 C per sample
2. Add 120 ul of binding solution to first strand reaction
3. Transfer to SNAP column and centrifuge at 13000×g for 20 s
4. Discard flow-through
5. Add 0.4 ml of cold 1× wash buffer to cartridge, centrifuge at 13000×g for 20 s. Discard flow-through. Repeat 3×.
6. Wash cartridge 2× with 400 ul Cold 70% ethanol. Discard flow-through.
7. centrifuge empty cartridge at 13,000×g for 1 min.
8. transfer cartridge to fresh tube, add 50 ul of preheated water and centrifuge for 20 s to elute DNA.

The cDNA was TdT tailed:

| | |
|---|---|
| H2O | 6.5 |
| 5× tailing buffer | 5 |
| 2 mM dCTP | 2.5 |
| SNAP purified cDNA | 10 |
| Final Volume | 24 |

Incubate for 2-3 min at 94 C, chill 1 min on ice.

Add 1 ul TdT, mix gently, incubate for 10 min at 37° C.

Heat inactivate TdT for 10 min at 65° C. Place on ice or store at −20° C. PCR of tailed cDNA:

| | |
|---|---|
| Q5 2× MasterMix | 25 |
| Primer (5 uM) | 15⁴ |
| Abridged Anchor Primer | 2 |
| (10 uM) | |
| dC-tailed cDNA | 5 |
| Final volume | 14 |

Add 0.5 ul of Taq DNA polymerase (0.5 U/ul) and mix.

Transfer tubes from ice to pre-equilibrated thermal cycler.

Cycling Conditions

| | | | |
|---|---|---|---|
| 98 C. | 30 | sec | 35× |
| 98 C. | 10 | sec | cycles |
| 55 C. | 20 | sec | |
| 72 C. | 20 | sec | |
| 72 C. | 5 | min | |

| | |
|---|---|
| 5 C. | Hold |

Primers used in PCR of tailed cDNA:

| Tdt tailed | | |
|---|---|---|
| cDNA | Primer used | |
| sgRNA2 | 5′RACE | agcgcccaatacgcaaaccgcc |
| | sgRNA2 | (SEQ ID NO: 31) |
| sgRNA2 | nested | gcgttggccgattcattaatgc |
| | sgRNA2 | (SEQ ID NO: 32) |

Primers used for sequencing of final 5′RACE PCR product:

| Primer used | |
|---|---|
| for sequencing | Primer sequence |
| nested sgRNA2 | gcgttggccgattcattaatgc |
| | (SEQ ID NO: 32) |

### LC-MS Analysis

Samples were analyzed on a Thermo Q-EXactive instrument with a Waters Acuity HPLC.

Mobile phase A) 200 mM hexafluoroisopropanol/8.15 mM triethylamine/0.75 uM EDTA, pH=8

Mobile phase B) MEOH

Column: Waters Acuity BEH 2.1×100 mm held at 70 C

Flow rate: 300 uL/min

Gradient conditions: Starting at 5% B and then 13% B at 0.6 min followed by linear ramping to 21% at 14 min, 90% at 18 min and then returning to 5% at 18.5 min.

The MS was operated in negative ion mode scanning from 700-2800 m/z.

Prior to all samples an 87 mer RNA standard was run to check LC-MS performance

### Results

Two sgRNAs with 5′A nucleotides were made (Table 11). The standard T7 polymerase promoter initiates transcription on G, and this promoter can be modified to force transcription to initiate on A (Table 12).

TABLE 11
sqRNA sequences initiating
with A nucleotides

| | |
|---|---|
| sgRNA1 | AUCAGAGGCCAAACCCUUCCGUUUUAGAGCU |
| | AGAAAUAGCAAGUUAAAAUAAGGCUAGUCCG |
| | UUAUCAACUUGAAAAAGUGGCACCGAGUCGG |
| | UGCUUUU (SEQ ID NO: 17) |
| sgRNA2 | ACUGAAUCGGAACAAGGCAAGUUUUAGAGCU |
| | AGAAAUAGCAAGUUAAAAUAAGGCUAGUCCG |
| | UUAUCAACUUGAAAAAGUGGCACCGAGUCGG |
| | UGCUUUU (SEQ ID NO: 18) |
| sgRNA3 | GGCCGAGAUGUCUCGCUCCGGUUUUAGAGCU |
| | AGAAAUAGCAAGUUAAAAUAAGGCUAGUCCG |
| | UUAUCAACUUGAAAAAGUGGCACCGAGUCGG |
| | UGCUUUU (SEQ ID NO: 76) |

Note:
The target specific sequence of the sgRNA is underlined.

TABLE 12
T7 polymerase promoter sequences.

| Standard | phi6.5 mut overlapped |
|---|---|
| T7 Promoter | T7 promoter |
| TAATACGACTCACTATAG | TAATACGACTCACTATAA |
| (SEQ ID NO: 19) | (SEQ ID NO: 20) |

Note:
Transcription initiation start is underlined

The sgRNA sequences were cloned into pUC57-kan vectors along with an upstream phi6.5 mut overlapped T7 promoter.

To determine if the sgRNAs produced via in vitro transcription did start with an A nucleotides as expected, 5′RACE was performed. Sequencing of the 5′RACE PCR products showed both RNAs started with an A nucleotide. These results combined with mass spec analysis showing the expected molecular weight indicate that use of the Phi6.5 mut overlapped promoter does force transcription to initiate on an A.

### Example 7

### SGRNA Template Preparation by PCR

The nature of PCR reaction allows to incorporate modifications at the end of the target sequence, it could be addition of non-templated sequence, or some tag (eg. biotin), and we thought that using primers with 2′OMe would generate PCR fragment carrying this NTP. The principle is outlined in FIGS. 4 and 5.

PCR reaction leads to blunt ended DNA fragment, but our experiments with synthetic oligoes showed that 5′ overhang on 3′ end of the template is beneficial, as such template allows for homogeneous sgRNA synthesis, without N+ subspecies. To generate such overhang we thought about incorporating restriction site for BbsI enzyme and include 2′OMe NTP at the BbsI cleavage site in such way, that after digest with BbsI, DNA fragment would contain 4 nt overhang with modified NTP at the end. This approach is illustrated in FIG. 5.

### Results

I. DNA Template Preparation by PCR

The primers used in the reactions are indicate in Table 13.

We performed first 4 small scale PCR reactions to select best DNA template for sgRNA synthesis which would eliminate n+x:

PCR reaction #1 would generate PCR fragment carrying 2′OMe A at the BbsI restriction digest site. Primer pair used for this reaction was Reverse primer 1 and Forward Primer

PCR reaction #2 would generate blunt PCR fragment with all natural dNTPs.

Primer pair used for this reaction was Reverse primer 3 and Forward Primer

PCR reaction #3 would generate PCR fragment with all natural dNTPs, introducing BbsI restriction digest site.

Primer pair used for this reaction was Reverse primer 2 and Forward Primer

PCR reaction #4 would generate blunt PCR fragment 2×2′OMe A at the 3′ end.

Primer pair used for this reaction was Reverse primer and Forward Primer

TABLE 13
LIST OF PRIMERS USED FOR
PCR REACTIONS

| Primer | |
|---|---|
| Name | Sequence |
| Reverse | AGGAAACAGCTATGACCATGC |
| primer 1 | TCGAGCCAAGCTCGGCGCGCC |
| | ATTGGGATGGAACGAAGACCm |
| | AmAAAGCACCGACTCGGTGCC |
| | (SEQ ID NO: 23) |
| Reverse | AGGAAACAGCTATGACCATGC |
| primer 2 | TCGAGCCAAGCTCGGCGCGCC |
| | ATTGGGATGGAACGAAGACCC |
| | AAAAGCACCGACTCGGTGCC |
| | (SEQ ID NO: 24) |
| Reverse | mAmAAAAGCACCGACTCGGTG |
| primer | CCAC (SEQ ID NO: 21) |
| Reverse | AAAAGCACCGACTCGGTGCC |
| primer 3 | (SEQ ID NO: 25) |
| Forward | TAACGCCAGGGTTTTCCCAGT |
| primer | CACG (SEQ ID NO: 22) |

PCR reactions were performed as follows:

All components should be mixed prior to use.

For each PCR reaction following components were mixed and transferred to individual PCR plate.

| | 50 μl | 100 × 100 ul |
|---|---|---|
| Component | Reaction | reactions |
| Q5 ® Hot Start High-Fidelity 2× | 25 | 5000 |
| Master Mix | | |
| 100 μM Forward Primer | 0.05 | 10 |
| 100 μM Reverse Primer | 0.05 | 10 |
| Template DNA, 1 ng/ul | 0.5 | 100 |
| Nuclease-Free Water | 24.4 | 4880 |

Collect all liquid to the bottom of the tube by a quick spin

Transfer PCR plates into PCR machine to start cycling reaction:

| STEP | TEMP | TIME | |
|---|---|---|---|
| Initial Denaturation | 98° C. | 30 | seconds |
| | 98° C. | 10 | seconds |
| 30 Cycles | 55° C. | 30 | seconds |
| | 72° C. | 10 | seconds |
| Final Extension | 72° C. | 2 | minutes |
| Hold | 4-10° C. | | |

After the completion of the cycles analyze results using agarose gel electrophoresis.

The PCR reaction was pooled and desalted using Vivaspin Turbo 15 ultrafiltration spin columns from Sartorius (30,000 MWCO PES).
Ultrafiltration spin columns were pre-rinsed using 10 ml of DNAse-RNAse free water. Water was added to the column and spun at 4400 g for 10 minutes and RT using a swing-bucket rotor in a centrifuge
2.5 ml of PCR reaction was added to spin columns.
Spin columns were spun at 4400 g for 3 minutes and RT using a swing-bucket rotor in a centrifuge
Flow-through was collected into separate tube and kept until it was confirmed that no PCR fragment was in flow-through
Filters were then washed with 10 mL dlH2O
Filters were spun at 4400 g for 3 minutes and RT using a swing-bucket rotor in a centrifuge
Flow-through was collected into separate tube and kept until it was confirmed that no PCR fragment was in flow-through
dlH2O wash is repeated twice as above
Filters were spun at 4400 g for 3 minutes and RT using a swing-bucket rotor in a centrifuge
Volume in each spin filter should be ˜50-250 uL
Collect and pooled together desalted PCR fragment solution.

300 ug of purified PCR 1 and 3 were digested using BbsI enzyme using following conditions:

| | PCR#1 | PCR#3 |
|---|---|---|
| reaction mix | UI | ul |
| total volume | 3,000.00 | 3,000.00 |
| buffer | 300.00 | 300.00 |
| (CutSmart) | | |
| DNA | 101.63 | 98.88 |
| Enzyme | 30.00 | 30.00 |
| (Bbsl) | | |
| H2O | 2,568.37 | 2,571.12 |

Reaction mix was incubated for 2 h at 37 C. After the completion of the incubation, reaction was analyzed using Novex TBE Gel, 4-20%, 15 well.

We found that PCR3 fragment (all natural dNTPs) was digested more efficiently than PCR1 (2×2′OMe incorporated into BbsI restriction site).

The PCR reaction was pooled and desalted using Vivaspin Turbo 15 ultrafiltration spin columns from Sartorius (30,000 MWCO PES) as described in Examples 7 and 8.

II. IVT Reactions and LC-MS Analysis

All 4 templates were used in IVT reaction and analyzed by LC-MS. The summary of the results is shown in Table 14.

TABLE 14

| Template | Expected | Observed | N+ |
|---|---|---|---|
| nickname | RNA size | RNA size | observed? |
| PCR#1 | 163 nt | 100 nt, 163 nt | Yes |
| PCR#1/Bbsl | 100 nt | 100 nt, 163 nt | Yes |
| PCR#2 | 100 nt | 100 nt | Yes |
| PCR#3 | 163 nt | 163 nt | Yes |
| PCR#3/Bbsl | 100 nt | 100 nt | Yes |
| PCR#4 | 100 nt | 100 nt | No |

Interesting, that use of PCR #4 (blunt fragment with 2′OMe at the 3′ end) as template, resulted in uniform product with expected size without formation of N+ products. In our previous experiment when synthetic oligoes were used as template, we observed reduced N+ formation when 4 nt overhang was formed, while use of blunt 3′ end resulted in formation of N+. Without being boundary by any theory, it is possible that by using 2×2′OMe A in our primer we generated 2 nt long single stranded overhang at 3′ end of the template and this, along with use of 2′OMe A, helped to eliminate N+ formation. This finding was confirmed when we repeated PCR reaction to generate new template. The resulting sgRNA also did not have any N+. As result, we chose this method of generation of the template for sgRNA IVT.

III. Alternative Conditions for PCR Reaction.

In initial PCR reactions we used Q5® Hot Start High-Fidelity 2× Master Mix in order to simplify the reaction set up. We realized that price of using separate components in the PCR reaction (i.e. Q5 polymerase, dNTP, PCR buffer) is lower than using 2× Master mix, and we set up the reactions accordingly. The following conditions were selected after series of the optimizations:

TABLE 15
ALTERNATIVE PCR CONDITIONS

| | 50 μl | 100 × 100 ul |
|---|---|---|
| Component | Reaction | reactions |
| Q5 Reaction Buffer | 5 | 1000 |
| dNT mix | 1 | 200 |
| 100 μM Forward Primer | 0.4 | 80 |
| 100 μM Reverse Primer | 0.4 | 80 |
| Template DNA, 1 ng/ul | 0.05 | 10 |
| Q5 Hot Start High-Fidelity DNA | 0.5 | 100 |
| Polymerase, 1 U | | |
| Nuclease-Free Water | 42.65 | 8530 |

The cycling conditions were same as above.

Using same method, templates for sgRNA2 and sgRNA1 were generated (FIGS. 13A-13C). Primers used to generate these templates are listed in Table 16.

TABLE 16
LIST OF PRIMERS USED FOR SGRNA2 AND
SGRNA1 PCR REACTIONS

| Primer | | Template |
|---|---|---|
| Name | Sequence | generated |
| Reverse | mAmAAAGCACCGACTCGGTGCCAC | sgRNA2, |
| primer | (SEQ ID NO: 21) | sgRNA1 |
| Forward | TAACGCCAGGGTTTTCCCAGTCACG | sgRNA2 |
| primer | (SEQ ID NO: 22) | |
| Forward | GTATGTTGTGTGGAATTGTGAGCG | sgRNA1 |
| primer 2 | (SEQ ID NO: 26) | |

In conclusion, PCR approach to generate DNA template for the sgRNA IVT is the way to introduce modified NTP at the 3′ end of DNA template. No restriction enzyme digest of the PCR fragment is needed as use of modified NTP in the reverse primer is introducing 2 nt overhang on the 3′ end of the template. When modified NTP is introduce in the template, significant reduction of the N+ amount RNA species is observed after IVT.

### Example 8

### Triphosphate RNA Production and Purification

### Materials and Methods

### DNA Template Production by PCR

All components should be mixed prior to use.

For each PCR reaction mix following components and gently mix.

| | | 100 × 100 ul |
|---|---|---|
| Component | 50 μl Reaction | reactions |
| Q5 Reaction Buffer | 5 | 1000 |
| dNTP mix | 1 | 200 |
| 100 μM Forward Primer | 0.25 | 50 |
| 100 μM Reverse Primer | 0.25 | 50 |
| Template DNA, 10 ng/ul | 0.05 | 10 |
| Q5 Hot Start High-Fidelity | 0.5 | 100 |
| DNA Polymerase, 1 U | | |
| Nuclease-Free Water | 42.95 | 8590 |

The following primers were used:

| Primer Name | Sequence |
|---|---|
| Reverse primer | mAmAAAGCACCGACTCGGTGCCAC |
| | (SEQ ID NO: 21) |
| Forward primer | TAACGCCAGGGTTTTCCCAGTCACG |
| | (SEQ ID NO: 22) |

Collect all liquid to the bottom of the tube by a quick spin

Aliquot reaction solution into PCR plate and transfer plates into PCR machine to start cycling reaction:

| STEP | TEMP | TIME |
|---|---|---|
| Initial Denaturation | 98° C. | 30 seconds |
| 60 Cycles | 98° C. | 10 seconds |
| | 55° C. | 30 seconds |
| | 72° C. | 10 seconds |
| Final Extension | 72° C. | 2 minutes |
| Hold | 4-10° C. | |

After the completion of the cycles analyze results using Bio-Rad Experion capillary electrophoresis on 1K DNA chips.

The PCR reaction was pooled and desalted using Vivaspin Turbo 15 ultrafiltration spin columns from Sartorius (30,000 MWCO PES) as described in Example 7.

Samples are tested for concentration and spectral purity (260/280 and 260/230) on a nanodrop instrument.

### In Vitro Transcription

Components are added in order and mixed.

| | 1× reaction | 20× Reaction |
|---|---|---|
| Nuclease free water | Up to 100 ul | 598.54 |
| 1 M Tris-HCl pH 8.0 (Sigma | 4 | 80 |
| T2694) | | |
| 1 M MgCl (Sigma, M1028) | 2.4 | 48 |
| GTP 100 mM (NEB, N0452B) | 6 | 120 |
| CTP 100 mM (NEB, N0450B | 6 | 120 |
| ATP 100 mM (NEB, N0451B) | 6 | 120 |
| PseudoUTP 100 mM (Trilink, | 6 | 120 |
| N1019) | | |
| DTT 1 M (Sigma, 43816) | 1 | 20 |
| Sperimidine 100 mM (Sigma, | 2 | 40 |
| S0266) | | |
| DNA Template (YD-30-YR84 | 2.5 ug (template | |
| 451 ng/ul) | produced 110 | |
| | by PCR) | |
| Pyrophosphatase 0.1 U/ul (NEB, | 2 | 40 |
| M2403B) | | |
| RNase Inhibitor 40 U/ul (NEB, | 2.5 | 50 |
| M0307B) | | |
| T7 RNA Polymerase 50 U/ul | 10 | 200 |
| (NEB, M0251B) | | |

Mix and Incubate at 37° C. for 17 hrs

| | | |
|---|---|---|
| LiCl 7.5 M (Ambion, AM9480) | 75 | 1500 |

Mix and Incubate at −20° C. for 1 h

After incubation at −20° C. in LiCl, centrifuge RNA for 45 minutes to pellet the RNA. Remove supernatant and wash the RNA pellet with 2 ml of 70% ethanol and centrifuge again for 45 minutes. Remove ethanol, let pellet air dry for 5 minutes and resuspend the RNA in nuclease free water.

### Reverse Phase Purification

HPLC or FPLC system that can monitor the presence of material at 260 nm and that is fitted with a fraction collector. This method uses an AKTA Explorer FPLC instrument with:
a. P-900 flow controller
b. UV-900 UV Detector collecting at 260 nm, 280 nm, and 230 nm.
c. pH/C-900 Conductivity and pH Detector
d. Frac-950 Fraction Collector
e. Unicorn Processing Software
f. TL105 column heater (Timberline Instruments, Boulder, Colo.).

HPLC column: Phenomenex Luna 5 μm C18(2) 100 Å (00B-4252-NO) (10×10 mm) (4 mL column)

Test the MilliQ water for endotoxin before making any of the buffers for the week. Must be below 0.005 EU/mL in order to use.

Buffer A: 0.1 M triethylammonium acetate (TEAA), pH 7.0 (Sigma, Part number: 90358-500 mL)
a. Add 50 ml TEAA.
b. Add 450 ml di-water.

Buffer B: 0.1 M TEAA, 50% acetonitrile, pH 7.0 (Sigma, Part number: 90358-500 mL)(Honeywell; Part number: BB017-4)
a. Add 50 ml TEAA.
b. Add 225 ml acetonitrile and 225 ml di-water.

Acetonitrile: 50% for column storing.

Acetic acid: 12% for column

0.1N NaOH HPLC system cleaning.

HPLC grade water.

Ethanol: 20% for long-term storage of HPLC system.

### Cleaning Method

To avoid any contamination both the system and column have to be cleaned prior to any purification.

Flush out the 50% Acetonitrile out of the system, column and buffer lines and replace with water.

Need to Sanitize/flush all lines including A11, B1, sample lines S1, S8, system with 0.1N NaOH and column on a separate machine with 12% Acetic acid and let it sit for a 2-3 hours to sanitize. A couple hours later, flush all the lines and system with water. Flush the machine and all lines with water as well Test the pH until it gets back down to 7.0. May use a little of Buffer A to bring the column back to pH 7.0 faster.

Reconnect both the column and the system back together.

Test the [system] (column in by-pass mode) and the [column+system] for endotoxin with the ENDOSAFE endotoxin testing system.

Put the system back into 50% Acetonitrile for overnight storage.

Pull out the tube of mRNA material out of the freezer to thaw overnight.

### Specification: Endotoxins

Apparatus: ENDOSAFE® MCS™—Multi cartridges system or ENDOSAFE®-PTS™ single cartridge system

Cartridges: Limulus Amebocyte Lysate Test Cartridges (Sensitivity 0.5-0.005 EU/mL) (Charles River; Product code: PTS20F or Reorder code: PTS20005F)

Specifications for devices: Endotoxin free

Specifications for System (HPLC/FPLC system): EU level <0.005 EU/mL

Specifications for the column: EU level <0.005 EU/mL

### Buffer Preparation

Make 500 mL of Buffer A and 250-500 mL of Buffer B fresh.

Test the MilliQ H2O for endotoxin first, then make buffers, once the tested endotoxin level is below 0.005 EU/mL.

Mobile Phases:

Buffer A: 0.1M Triethylammonium acetate (TEAA), pH 7.0

For 500 mL: 50 mL TEAA and 450 mL DI-water.

Buffer B: 0.1M TEAA, 50% Acetonitrile (HPLC grade), pH 7.0

For 500 mL: 50 mL TEAA and 225 mL acetonitrile and 225 mL DI-water.

### Purification Method

Mobile Phases:

Buffer A: 100 mM TEAA in di-Water

Buffer B: 100 mM TEAA in 50% Acetonitrile (HPLC grade)

Apparatus: AKTA purifier or AKTA explorer

Column: Phenomenex Luna C18(2) (50×10 mm) (4 mL)

Column Pressure limit: 10 MPa

Column Position: 8

Method: Phenomenex Luna 48 ml RP

Injection flowrate: 5 mL/min

Elution flowrate: 50 mL/min

Column heating: set at 65° C.

Wavelength: 230 nm, 260 nm and 280 nm

Equilibration: 8 CV—9% Buffer B

Dilute mRNA 1:1 with 9% Buffer B before injecting to the column.

Sample Inlet: S1

Reverse Phase Purification of 5 mg mRNA on a 5 mL Column

Set column oven to 65° C.

Dilute mRNA 1:1 with 9% Buffer B before injecting to the column.

Set the flow rate to 5 ml/min.

Equilibrate column with 8 column void volumes of 9% buffer B.

Load RNA onto column at 5 ml/min using S1 inlet.

Wash the column with 3 column void volumes of 9% buffer B

Run a linear gradient from 0% to 9% buffer B over 5 column void volumes

Run a linear gradient from 9% to 35% buffer B over 27 column void volumes

Collect 10 ml fractions (Specifications: UV260>100 mAU)

### LC-MS Analysis

Samples were analyzed on a Thermo Q-EXactive instrument with a Waters Acuity HPLC.

Mobile phase A) 200 mM hexafluoroisopropanol/8.15 mM triethylamine/0.75 uM EDTA, pH=8

Mobile phase B) MEOH

Column: Waters Acuity BEH 2.1×100 mm held at 70 C

Flow rate: 300 uL/min

Gradient conditions: Starting at 5% B and then 13% B at 0.6 min followed by linear ramping to 21% at 14 min, 90% at 18 min and then returning to 5% at 18.5 min.

The MS was operated in negative ion mode scanning from 700-2800 m/z.

Prior to all samples an 87 mer RNA standard was run to check LC-MS performance.

### Results

The DNA template for sgRNA2 was generated via PCR off plasmid DNA as starting template. An IVT reaction was used to produce sgRNA2 RNA from PCR DNA template. Fractions collected from the purification were assessed by nanodrop, Bio Rad Experion, Mass spec, and SEC.

sgRNA2 triphosphate RNA was successfully purified via reverse phase column purification. The final purified RNA material was aliquoted into 150 ug aliquots and stored at −80° C.

### Example 9

### Hydroxyl RNA Production

### Materials and Methods

### DNA Template Production by PCR

All components should be mixed prior to use.

For each PCR reaction mix following components and gently mix.

| | 50 μl | 100 × 100 ul |
|---|---|---|
| Component | Reaction | reactions |
| Q5 Reaction Buffer | 5 | 1000 |
| dNTP mix | 1 | 200 |
| 100 μM Forward Primer | 0.25 | 50 |
| 100 μM Reverse Primer | 0.25 | 50 |
| Template DNA, 10 ng/ul | 0.05 | 10 |
| Q5 Hot Start High-Fidelity | 0.5 | 100 |
| DNA Polymerase, 1 U | | |
| Nuclease-Free Water | 42.95 | 8590 |

The following primers were used:

| Primer Name | Sequence |
|---|---|
| Reverse primer | mAmAAAGCACCGACTCGGTGCCAC |
| | (SEQ ID NO: 21) |
| Forward primer | TAACGCCAGGGTTTTCCCAGTCACG |
| | (SEQ ID NO: 22) |

Collect all liquid to the bottom of the tube by a quick spin.

Aliquot reaction solution into PCR plate and transfer plates into PCR machine to start cycling reaction:

| STEP | TEMP | TIME | |
|---|---|---|---|
| Initial Denaturation | 98° C. | 30 | seconds |
| | 98° C. | 10 | seconds |
| 30 Cycles | 55° C. | 30 | seconds |
| | 72° C. | 10 | seconds |
| Final Extension | 72° C. | 2 | minutes |
| Hold | 4-10° C. | | |

After the completion of the cycles analyze results using Bio-Rad Experion capillary electrophoresis on 1K DNA chips.

The PCR reaction was pooled and desalted using Vivaspin Turbo 15 ultrafiltration spin columns from Sartorius (30,000 MWCO PES) as described in Example 7.

Samples are tested for concentration and spectral purity (260/280 and 260/230) on a nanodrop instrument.

### In Vitro Transcription

Components are added in order and mixed.

| | 1× reaction | 40× Reaction |
|---|---|---|
| Nuclease free water | Up to 100 ul | 1343.26 |
| 1 M Tris-HCl pH 8.0 (Sigma | 4 | 160 |
| T2694) | | |
| 1 M MgCl (Sigma, M1028) | 2.4 | 96 |
| GTP 100 mM (NEB, N0452B) | 6 | 240 |
| CTP 100 mM (NEB, N0450B | 6 | 240 |
| ATP 100 mM (NEB, N0451B) | 6 | 240 |
| PseudoUTP 100 mM (Trilink, | 6 | 240 |
| N1019) | | |
| DTT 1 M (Sigma, 43816) | 1 | 40 |
| Sperimidine 100 mM (Sigma, | 2 | 80 |
| S0266) | | |
| DNA Template (UF-20-BB11 | 2.5 ug (template produced | 740.74 |
| 270 ng/ul) | by PCR) | |
| Pyrophosphatase 0.1 U/ul (NEB, | 2 | 80 |
| M2403B) | | |
| RNase Inhibitor 40 U/ul (NEB, | 2.5 | 100 |
| M0307B) | | |
| T7 RNA Polymerase 50 U/ul | 10 | 400 |
| (NEB, M0251B) | | |

Mix and Incubate at 37° C. for 17 hrs

| | | |
|---|---|---|
| LiCl 7.5 M (Ambion, AM9480) | 75 | 3000 |

Mix and Incubate at −20° C. for 1 h

After incubation at −20° C. in LiCl, centrifuge RNA for 45 minutes to pellet the RNA. Remove supernatant and wash the RNA pellet with 2 ml of 70% ethanol and centrifuge again for 45 minutes. Remove ethanol, let pellet air dry for 5 minutes and resuspend the RNA in nuclease free water.

### Dephosphorylation

Dephosphorylation reaction was done according to the table below. Components are mixed together and incubated @ 37 C for 2 h.

TABLE 17
PROTOCOL FOR DEPHOSPHORYLATION
OF 5′-ENDS OF sgRNA
SYNTHESIZED BY IN VITRO
TRANSCRIPTION

| 1 | Prepare reaction as follows: | | |
|---|---|---|---|
| | IVT synthesized sgRNA | 1 | mg |
| | 10× CutSmart Buffer | 7.5 | ml |
| | CIP (10 U/μl) NEB; M0290L | 6500 | units |
| | RNAse inhibitor (40 U/ul) | 1875 | ul |

| | |
|---|---|
| RNAse free H2O | Up to 75 mL |

| | |
|---|---|
| 2 | Incubate at 37° C. with gentle shaking for 2 hr, |
| | then stored at −20° C. until |
| | RP-HPLC purification |

### Reverse Phase Purification

HPLC or FPLC system that can monitor the presence of material at 260 nm and that is fitted with a fraction collector. This method uses an AKTA Explorer FPLC instrument with:
a. P-900 flow controller
b. UV-900 UV Detector collecting at 260 nm, 280 nm, and 230 nm.
c. pH/C-900 Conductivity and pH Detector
d. Frac-950 Fraction Collector
e. Unicorn Processing Software
f. TL105 column heater (Timberline Instruments, Boulder, Colo.).

HPLC column: Phenomenex Luna 5 μm C18(2) 100 Å (00B-4252-NO) (10×10 mm) (4 mL column)

Test the MilliQ water for endotoxin before making any of the buffers for the week. Must be below 0.005 EU/mL in order to use.

Buffer A: 0.1 M triethylammonium acetate (TEAA), pH 7.0 (Sigma, Part number: 90358-500 mL)
a. Add 50 ml TEAA.
b. Add 450 ml di-water.

Buffer B: 0.1 M TEAA, 50% acetonitrile, pH 7.0 (Sigma, Part number: 90358-500 mL)(Honeywell; Part number: BB017-4)
a. Add 50 ml TEAA.
b. Add 225 ml acetonitrile and 225 ml di-water.

Acetonitrile: 50% for column storing.

Acetic acid: 12% for column

0.1N NaOH HPLC system cleaning.

HPLC grade water.

Ethanol: 20% for long-term storage of HPLC system.

### Cleaning Method

To avoid any contamination both the system and column have to be cleaned prior to any purification.

Flush out the 50% Acetonitrile out of the system, column and buffer lines and replace with water.

Need to Sanitize/flush all lines including A11, B1, sample lines S1, S8, system with 0.1N NaOH and column on a separate machine with 12% Acetic acid and let it sit for a 2-3 hours to sanitize. A couple hours later, flush all the lines and system with water. Flush the machine and all lines with water as well Test the pH until it gets back down to 7.0. May use a little of Buffer A to bring the column back to pH 7.0 faster.

Reconnect both the column and the system back together.

Test the [system] (column in by-pass mode) and the [column+system] for endotoxin with the ENDOSAFE endotoxin testing system.

Put the system back into 50% Acetonitrile for overnight storage.

Pull out the tube of mRNA material out of the freezer to thaw overnight.

### Specification: Endotoxins

Apparatus: ENDOSAFE® MCS™—Multi cartridges system or ENDOSAFE®-PTS™ single cartridge system

Cartridges: Limulus Amebocyte Lysate Test Cartridges (Sensitivity 0.5-0.005 EU/mL) (Charles River; Product code: PTS20F or Reorder code: PTS20005F)

Specifications for devices: Endotoxin free

Specifications for System (HPLC/FPLC system): EU level <0.005 EU/mL

Specifications for the column: EU level <0.005 EU/mL

### Buffer Preparation

Make 500 mL of Buffer A and 250-500 mL of Buffer B fresh.

Test the MilliQ H2O for endotoxin first, then make buffers, once the tested endotoxin level is below 0.005 EU/mL.

Mobile Phases:

Buffer A: 0.1M Triethylammonium acetate (TEAA), pH 7.0

For 500 mL: 50 mL TEAA and 450 mL DI-water.

Buffer B: 0.1M TEAA, 50% Acetonitrile (HPLC grade), pH 7.0

For 500 mL: 50 mL TEAA and 225 mL acetonitrile and 225 mL DI-water.

### Purification Method

Mobile Phases:

Buffer A: 100 mM TEAA in di-Water

Buffer B: 100 mM TEAA in 50% Acetonitrile (HPLC grade)

Apparatus: AKTA purifier or AKTA explorer

Column: Phenomenex Luna C18(2) (50×10 mm) (4 mL)

Column Pressure limit: 10 MPa

Column Position: 8

Method: Phenomenex Luna 48 ml RP

Injection flowrate: 5 mL/min

Elution flowrate: 50 mL/min

Column heating: set at 65° C.

Wavelength: 230 nm, 260 nm and 280 nm

Equilibration: 8 CV—9% Buffer B

Dilute mRNA 1:1 with 9% Buffer B before injecting to the column.

Sample Inlet: S1

Reverse Phase Purification of 5 mg mRNA on a 4 mL Column

Set column oven to 65° C.

Dilute mRNA 1:1 with 9% Buffer B before injecting to the column.

Set the flow rate to 5 ml/min.

Equilibrate column with 8 column void volumes of 9% buffer B.

Load RNA onto column at 5 ml/min using S1 inlet.

Wash the column with 3 column void volumes of 9% buffer B

Run a linear gradient from 0% to 9% buffer B over 5 column void volumes

Run a linear gradient from 9% to 35% buffer B over 27 column void volumes

Collect 10 ml fractions (Specifications: UV260>100 mAU)

### LC-MS Analysis

Samples were analyzed on a Thermo Q-EXactive instrument with a Waters Acuity HPLC.

Mobile phase A) 200 mM hexafluoroisopropanol/8.15 mM triethylamine/0.75 uM EDTA, pH=8

Mobile phase B) MEOH

Column: Waters Acuity BEH 2.1×100 mm held at 70 C

Flow rate: 300 uL/min

Gradient conditions: Starting at 5% B and then 13% B at 0.6 min followed by linear ramping to 21% at 14 min, 90% at 18 min and then returning to 5% at 18.5 min.

The MS was operated in negative ion mode scanning from 700-2800 m/z.

Prior to all samples an 87 mer RNA standard was run to check LC-MS performance

### Results

The DNA template for sgRNA2 was generated via PCR of plasmid pUC57-Kan_sgRNA2. An IVT reaction was used to produce sgRNA2 RNA from the DNA template. An aliquot was saved as a pre-purification sample and then 5 mg of the RNA was dephosphorylated using calf intestinal alkaline phosphatase (CIP). The dephosphorylation reaction was split into 5 separate reactions using decreasing amounts of CIP to determine if the amount of CIP used could be lowered from the initial 7.5 U/ug sgRNA. After the dephosphorylation reaction, 30 ug of RNA from each reaction was purified using Qiagen RNeasy minielute cleanup columns and assessed for dephosphorylation using the mass spec. The sgRNA in each reaction was fully dephosphorylated the RNA, even the lowest concentration of 1 U CIP per ug sgRNA.

The dephosphorylated RNA cleaned up by RNeasy columns was combined and registered as the pre purified hydroxyl sample. The remaining dephosphorylation reactions were combined and sgRNA was purified using reverse phase column chromatography. The CIP reactions were directly applied to the column there was no need for an intermediate cleanup step. Fractions collected from the purification were assessed by nanodrop, Bio Rad Experion, Mass spec, and SEC.

The Bio Rad Experion showed that fractions A9-B10 contain the majority of the product. The concentration of each fraction was determined by nanodrop, this confirmed that the majority of the product is in fractions A9-B10. The mass spec of the fractions showed that fractions A10-B8 have purities higher than 66%. Fractions B7-B3 and the pre purified all have lower purity levels and should not be used for pooling. Based on the analytics performed, fractions A10-B9 were pooled and buffer exchanged using vivaspin columns. The final purified sample was compared to the pre-purifed RNA on BioRad Experion, Mass spec, and THP-1 assay. sgRNA2 hydroxyl RNA was successfully purified via reverse phase purification. The final purified RNA material was aliquoted into 150 ug aliquots and stored at −80° C.

### SEQUENCE LISTING

TABLE 18
SEQUENCE IDENTIFICATION NUMBERS

| SEQ ID | | |
|---|---|---|
| NO. | Polynucleotide | Sequence |
| 1 | T7 RNA polymerase | TAATACGACTCACTATA |
| | promoter | |
| 2 | T3 RNA polymerase | AATTAACCCTCACTAAAG |
| | promoter | |
| 3 | SP6 RNA polymerase | ATTTAGGTGACACTATAG |
| | promoter | |
| 4 | Syn5 RNA polymerase | ATTGGGCACCCGTAA |
| | promoter | |
| 5 | tracrRNA | GTTTTAGAGCTAGAAATAGCAAGTTAAAAT |
| | | AAGGCTAGTCCGTTATCAACTTGAAAAAGT |
| | | GGCACCGAGTCGGTGCTTTT |
| 6 | SEQ ID NO: 90 of WO | GGGNNNNNNNNNNNNNNNNNNNNNNNNNGU |
| | 2015/006747 (Moderna | UUUAGAGCUAGAAAUAGCAAGUUAAAAUAA |
| | Therapeutics). | GGCUAGUCCGUUAUCAACUUGAAAAAGUGG |
| | | CACCGAGUCGGUGGUGC |
| 7 | Exemplary sgRNA | NNNNNNNNNNNNNNNNNNNGUUUUAGAGCU |
| | molecule | AGAAAUAGCAAGUUAAAAUAAGGCUAGUCC |
| | | GUUAUCAACUUGAAAAAGUGGCACCGAGUC |
| | | GGUGC |
| 8 | T7 terminator sequence | GCTAGTTATTGCTCAGCGG |
| 9 | Hepatitis delta virus | GGCCGGCATGGTCCCAGCCTCCTCGCTGGC |
| | (HDV) ribozyme | GCCGGCTGGGCAACATTCCGAGGGGACCGT |
| | | CCCCTCGGTAATGGCGAATGGGACG |
| 10 | T7 RNA polymerase | GGATCCGGAGGCCGGAGAATTG |
| | promoter upstream | |
| | enhancer sequence | |
| 11 | Template | AAAAGCACCGACTCGGTGCCACTTTTTCAA |
| | | GTTGATAACGGACTAGCCTTATTTTAACTT |
| | | GCTATTTCTAGCTCTAAAACTGAAGAAGAT |
| | | GGTGCGCTCCTATAGTGAGTCGTATTACAA |
| | | TTCTCCGGCCTCCGGATCC |
| 12 | template biotin | /5-bio/ |
| | | AAAAGCACCGACTCGGTGCCACTTTTTCAA |
| | | GTTGATAACGGACTAGCCTTATTTTAACTT |
| | | GCTATTTCTAGCTCTAAAACTGAAGAAGAT |
| | | GGTGCGCTCCTATAGTGAGTCGTATTACAA |
| | | TTCTCCGGCCTCCGGATCC |
| 13 | non-template | GGATCCGGAGGCCGGAGAATTGTAATACGA |
| | | CTCACTATAGGAGCGCACCATCTTCTTCAG |
| | | TTTTAGAGCTAGAAATAGCAAGTTAAAATA |
| | | AGGCTAGTCCGTTATCAACTTGAAAAAGTG |
| | | GCACCGAGTCGGTGCTTTT |
| 14 | non-template minus | GGATCCGGAGGCCGGAGAATTGTAATACGA |
| | 4T | CTCACTATAGGAGCGCACCATCTTCTTCAG |
| | | TTTTAGAGCTAGAAATAGCAAGTTAAAATA |
| | | AGGCTAGTCCGTTATCAACTTGAAAAAGTG |
| | | GCACCGAGTCGGTGC |
| 15 | template 2′Ome | mAmAAAGCACCGACTCGGTGCCACTTTTTC |
| | | AAGTTGATAACGGACTAGCCTTATTTTAAC |
| | | TTGCTATTTCTAGCTCTAAAACTGAAGAAG |
| | | ATGGTGCGCTCCTATAGTGAGTCGTATTAC |
| | | AATTCTCCGGCCTCCGGATCC |
| | | GGAGCGCACCATCTTCTTCA |
| 16 | crRNA, specifically | GGAGCGCACCATCTTCTTCA |
| | homologous to GFP | |
| | RNA target to be | |
| | cleaved | |
| 27 | Phi 2.5 overlapping | TAATACGACTCACTATT |
| | promoter | |
| 28 | AC15/C26 mutA promoter | TAATACGACTCACAATC |
| 29 | A6/B1 mutA promoter | TAATACGACTCACTCCG |
| 30 | phi 9 (A-15C) promoter | TACTACGACTCACTATA |

TABLE 19
EXEMPLARY SGRNA SEQUENCES

| Type | | 5′-3′ SEQUENCE |
|---|---|---|
| | | sgRNA |
| II | spCas9 | (N15- |
| | | 25)GUUUUAGAGCUAUGCUGgaaaCAG |
| | | CAUAGCAAGUUAAAAUAAGGCUAGUCC |
| | | GUUAUCAACUUGAAAAAGUGGCACCGA |
| | | GUCGGUGCUUU (SEQ ID NO: 33) |
| | nmCas9 | (N15- |
| | | 25)GUUGUAGCUCCCUUUCUCAUUUCG |
| | | gaaaCGAAAUGAGAACCGUUGCUACAA |
| | | UAAGGCCGUCUGAAAAGAUGUGCCGCA |
| | | ACGCUCUGCCCCUUAAAGCUUCUGCUU |
| | | UAAGGGGCAUCGUUUA (SEQ ID |
| | | NO: 34) |
| | saCas9 | (N15- |
| | | 25)GUUUUAGUACUCUGUAAUUUgaaa |
| | | AAAUUACAGAAUCUACUAAAACAAGGC |
| | | AAAAUGCCGUGUUUAUCUCGUCAACUU |
| | | GUUGGCGAGAUUU (SEQ ID NO: |
| | | 35) |
| | st1Cas9 | (N15- |
| | | 25)GUUUUUGUACUCUCAAGAUUcaau |
| | | AAUCUUGCAGAAGCUACAAAGAUAAGG |
| | | CUUCAUGCCGAAAUCAACACCCUGUCA |
| | | UUUUAUGGCAGGGUGUUU (SEQ ID |
| | | NO: 36) |
| | st3Cas9 | (N15- |
| | | 25)GUUUUAGAGCUGUGUUGUUUgtta |
| | | AAACAACACAGCGAGUUAAAAUAAGGC |
| | | UUAGUCCGUACUCAACUUGAAAAGGUG |
| | | GCACCGAUUCGGUGUUU (SEQ ID |
| | | NO: 37) |
| | cjCas9 | (N15- |
| | | 25)GUUUUAGUCCCUgaaaAGGGACUA |
| | | AAAUAAAGAGUUUGCGGGACUCUGCGG |
| | | GGUUACAAUCCCCUAAAACCGCUUU |
| | | (SEQ ID NO: 38) |
| | GeoCas9 | (N15- |
| | | 25)GUCAUAGUUCCCCUGAgaaaUCAG |
| | | GGUUACUAUGAUAAGGGCUUUCUGCCU |
| | | AAGGCAGACUGACCCGCGGCGUUGGGG |
| | | AUCGCCUGUCGCCCGCUUUUGGCGGGC |
| | | AUUCCCCAUCCUU (SEQ ID NO: |
| | | 39) |
| | FnCas9 | (N15- |
| | | 25)GUUUCAGUUGCGCCgaaaGGCGCU |
| | | CUGUAAUCAUUUAAAAGUAUUUUGAAC |
| | | GGACCUCUGUUUGACACGUCUG |
| | | (SEQ ID NO: 40) |

TABLE 20
EXEMPLARY SGRNA SEQUENCES

| Type | | 5′ handle (5′-3′ SEQUENCE) |
|---|---|---|
| V | fnCas12a | UAAUUUCUACUGUUGUAGAU(N15-25) |
| | | (SEQ ID NO: 41) |
| | AsCas12a | UAAUUUCUACUCUUGUAGAU(N15-25) |
| | | (SEQ ID NO: 42) |
| | Lb2Cas12a | UAAUUUCUACUAUUGUAGAU(N15-25) |
| | | (SEQ ID NO: 43) |
| | CMtCas12a | UAAUUUCUACUCUUUGUAGAU(N15-25) |
| | | (SEQ ID NO: 44) |
| | EeCas12a | UAAUUUCUACUUUGUAGAU(N15-25) |
| | | (SEQ ID NO: 45) |
| | MbCas12a | UAAUUUCUACUGUUUGUAGAU(N15-25) |
| | | (SEQ ID NO: 46) |
| | PdCas12a | UAAUUUCUACUUCGGUAGAU(N15-25) |
| | | (SEQ ID NO: 47) |
| | AacCas12b | GGUCUAGAGGACAGAAUUUUUCAACGGGU |
| | | GUGCCAAUGGCCACUUUCCAGGUGGCAAA |
| | | GCCCGUUGAGCUUCUCAAAUCUGAGAAGU |
| | | GGCAC(N15-25) (SEQ ID NO: 48) |
| VI | LshCas13a | GGCCACCCCAAUAUCGAAGGGGACUAAAA |
| | | C(N15-25) (SEQ ID NO: 49) |
| | AaCas13b | AAUUCUACUCUUGUAGAU(N15-25) |
| | | (SEQ ID NO: 50) |
| | PspCas13b | (N15- |
| | | 25)GUUGUGGAAGGUCCAGUUUUGGGGGC |
| | | UAUUACAACA (SEQ ID NO: 51) |

### REFERENCES

With respect to general information on CRISPR-Cas systems, components thereof, and delivery of such components, the teachings of the following documents may be useful:

U.S. Pat. Nos. 8,697,359, 8,771,945, 8,795,965, 8,865,406, 8,871,445, 8,889,356, 8,889,418 and 8,895,308.

U.S. Patent Publications US 2014/0310830 A1, US 2014/0287938 A1, US 2014/0273234 A1, US 2014/0273232 A1, US 2014/0273231 A1, US 2014/0256046 A1, US 2014/0248702 A1, US 2014/0242700 A1, US 2014/0242699 A1, US 2014/0242664 A1, US 2014/0234972 A1, US 2014/0227787 A1, US 2014/0189896 A1, US 2014/0186958, US 2014/0186919 A1, US 2014/0186843 A1, US 2014/0179770 A1, US 2014/0179006 A1 and US 2014/0170753.

European Patent Applications EP 2 771 468 A1, EP 2 764 103 A1, and EP 2 784 162 A1.

PCT Patent Publications WO 2014/093661, WO 2014/093694, WO 2014/093595, WO 2014/093718, WO 2014/093709, WO 2014/093622, WO 2014/093635, WO 2014/093655, WO 2014/093712, WO 2014/093701, WO 2014/018423, WO 2014/204723, WO 2014/204724, WO 2014/204725, WO 2014/204726, WO 2014/204727, WO 2014/204728, and WO 2014/204729.

PCT Patent Application Nos: PCT/US2014/041803, PCT/US2014/041800, PCT/US2014/041809, PCT/US2014/041804, PCT US2014/041806, PCT US2014/041808, PCT/US2014/62558 and PCT/US2014/41806.

Canver et al. (Nov. 12, 2015) BCL 11 A enhancer dissection by Cas9-mediated in situ saturating mutagenesis, Nature 527(7577): 192-7.

Chen et al. (Mar. 12, 2015) Genome-wide CRISPR Screen in a Mouse Model of Tumor Growth and Metastasis, Cell 160, 1246-1260 (multiplex screen in mouse) relates to multiplex screening by demonstrating that a genome-wide in vivo CRISPR-Cas9 screen in mice reveals genes regulating lung metastasis.

Chylinski et al. (2013) RNA Biology 10: 5, 727-737, described exemplary naturally occurring Cas9 molecules, from many cluster bacterial families.

Cong et al. (Feb. 15, 2013) Multiplex genome engineering using CRISPR/Cas systems, Science 339(6121): 819-23, engineered type II CRISPR/Cas systems for use in eukaryotic cells based on both Streptococcus thermophilus Cas9 and also Streptoccocus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can mediate mammalian genome cleavage. Importantly, it can be envisaged that several aspects of the CRISPR/Cas system can be further improved to increase its efficiency and versatility.

Doench et al. (2014) Rational design of highly active sgRNAs for CRISPR-Cas9-mediated gene inactivation, Nature Biotechnology, doi: 10.1038/nbt.3026, created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and provided an on-line tool for designing sgRNAs.

Hsu et al. (2013) DNA targeting specificity of RNA-guided Cas9 nucleases, Nature Biotechnol. doi: 10.1038/nbt.2647, characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. Hsu et al. found that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and sgRNA can be titrated to minimize off-target modification. Additionally, to facilitate mammalian genome engineering applications, the authors reported providing a web-based software tool to guide the selection and validation of target sequences as well as off-target analyses.

Hsu et al. (5 Jun. 2014) Development and Applications of CRISPR-Cas9 for Genome Engineering, Cell 157: 1262-1278, is a review article that discusses generally CRISPR-Cas9 history from yogurt to genome editing, including genetic screening of cells.

Jiang et al. (March 2013) RNA-guided editing of bacterial genomes using CRISPR-Cas systems, Nature Biotechnol. 31(3): 233-9 used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA: Cas9-directed cleavage at the targeted genomic site to kill unmutated cells and circumvents the need for selectable markers or counter-selection systems, The study reported reprogramming dual-RNA: Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering in Streptococcus pneumoniae, nearly 100% of cells that were recovered using the described approach contained the desired mutation, and in Escherichia coli, 65% that were recovered contained the mutation.

Jinek et al. (2012) A Programmable Dual-RNA-Guided DNA Endonuclease in Adaptive Bacterial Immunity. Science 337: 816-821.

Konermann et al. (22 Aug. 2013) Optical control of mammalian endogenous transcription and epigenetic states, Nature, 500(7463): 472-6. doi: 10.1038/Nature 12466, addressed the need in the art for versatile and robust technologies that enable optical and chemical modulation of DNA-binding domains based CRISPR Cas9 enzyme and Transcriptional Activator Like Effectors.

Konermann et al. (29 Jan. 2015) Genome-scale transcriptional activation by an engineered CRISPR-Cas9 complex, Nature 517(7536): 583-8, doi: 10.1038/nature14136, discuss the ability to attach multiple effector domains, e.g., transcriptional activator, functional and epigenomic regulators at appropriate positions on the guide such as stem or tetraloop with and without linkers.

Larson et al. (2013) CRISPR interference (CRISPRi) for sequence-specific control of gene expression. Nature Protocols 8: 2180-2196.

Nishimasu et al. (27 Aug. 2015) Crystal Structure of Staphylococcus aureus Cas9, Cell 162, 1113-1126, reported the crystal structure of Streptococcus pyogenes Cas9 in complex with sgRNA and its target DNA at 2.5 A° resolution. The structure revealed a bi-lobed architecture composed of target recognition and nuclease lobes, accommodating the sgRNA: DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.

Nishimasu et al. (27 Feb. 2014) Crystal structure of cas9 in complex with guide RNA and target DNA. Cell 156(5): 935-49, reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.

Parnas et al. (30 Jul. 2015) A Genome-wide CRISPR Screen in Primary Immune Cells to Dissect Regulatory Networks, Cell 162, 675-686, introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.

Piatt et al. (2014) CRISPR-Cas9 Knockin Mice for Genome Editing and Cancer Modeling, Cell 159(2): 440-455, DOI: 10.1016/j.cell.2014.09.014 established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.

Ramanan et al. (2 Jun. 2015) CRISPR/Cas9 cleavage of viral DNA efficiently suppresses hepatitis B virus, Scientific Reports 5: 10833. doi: 10.1038/srep10833 taught that HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.

Ran et al. (Apr. 9, 2015) In vivo genome editing using Staphylococcus aureus Cas9, Nature 520(7546): 186-91 (published online 1 Apr. 2015).

Ran et al. (28 Aug. 2013) Double Nicking by RNA-Guided CRISPR Cas9 for Enhanced Genome Editing Specificity, Cell, pii: S0092-8674(13)01015-5 [Ran et al. (2013-A)], described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.

Ran et al. (November 2013) Genome engineering using the CRISPR-Cas9 system. Nature Protocols 8(II): 2281-308 [Ran et al. (2013-B)], described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors' experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.

Shalem et al. (12 Dec. 2013) Genome-Scale CRISPR-Cas9 Knockout Screening in Human Cells. Science [Epub ahead of print], described a new way to interrogate gene function on a genome-wide scale. Their studies showed that delivery of a genome-scale CRISPR-Cas9 knockout (GeC O) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED 12 as well as novel hits NF2, CUL3, TADA2B, and TADAL The authors observed a high level of consistency between independent guide RNAs targeting the same gene and a high rate of hit confirmation, and thus demonstrated the promise of genome-scale screening with Cas9.

Shalem et al. (May 2015) High-throughput functional genomics using CRISPR-Cas9, Nature Reviews Genetics 16, 299-311, described ways in which catalytically inactive Cas9 (dCas9) fusions are used to synthetically repress (CRISPRi) or activate (CRISPRa) expression, showing, advances using Cas9 for genome-scale screens, including arrayed and pooled screens, knockout approaches that inactivate genomic loci and strategies that modulate transcriptional activity.

Slaymaker et al. (2015) Science Express, at Science DOI: 10.1126/science.aad5227, reported the use of structure-guided protein engineering to improve the specificity of Streptococcus pyogenes Cas9 (SpCas9). The authors developed “enhanced specificity” SpCas9 (eSpCas9) variants which maintained robust on-target cleavage with reduced off-target effects.

Swiech et al. (2014) In vivo interrogation of gene function in the mammalian brain using CRISPR-Cas9, Nature Biotechnol., doi: 10.1038/nbt.3055, demonstrated that AAV-mediated SpCas9 genome editing can enable reverse genetic studies of gene function in the brain.

Tsai et al. (2014) Dimeric CRISPR A-guided FokI nucleases for highly specific genome editing. Nature Biotechnology 32(6): 569-77, can be considered in the practice of the invention.

Wang et al. (3 Jan. 2014) Genetic screens in human cells using the CRISPR/Cas9 system, Science 343(6166): 80-84. doi: 10.1126/science.1246981, describes a pooled, loss-of-function genetic screening approach suitable for both positive and negative selection that uses a genome-scale lentiviral single guide RNA (sgRNA) library.

Wang et al. (9 May 2013) One-Step Generation of Mice Carrying Mutations in Multiple Genes by CRISPR/Cas-Mediated Genome Engineering, Cell 153(4): 910-8, used the CRISPR/Cas system for the one-step generation of mice carrying mutations in multiple genes which were traditionally generated in multiple steps by sequential recombination in embryonic stem cells and/or time-consuming intercrossing of mice with a single mutation.

Wu et al. (20 Apr. 2014) Genome-wide binding of the CRISPR endonuclease Cas9 in mammalian cells. Nature Biotechnol. doi: 10.1038/nbt.2889, mapped genome-wide binding sites of a catalytically inactive Cas9 (dCas9) from Streptococcus pyogenes loaded with sgRNAs in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences. The authors showed that targeted sequencing of 295 dCas9 binding sites in mESCs transfected with catalytically active Cas9 identified only one site mutated above background levels.

Xu et al. (August 2015) Sequence determinants of improved CRISPR sgRNA design, Genome Research 25, 1147-1157, assessed the DNA sequence features that contribute to single guide RNA (sgRNA) efficiency in CRISPR-based screens. The authors explored the efficiency of CRISPR/Cas9 knockout and nucleotide preference at the cleavage site. The authors found that the sequence preference for CRISPRi/a is substantially different from that for CRISPR Cas9 knockout.

Zetsche et al. (February 2015) A split-Cas9 architecture for inducible genome editing and transcription modulation, Nature Biotechnol. 33(2): 139-42, demonstrates that the Cas9 enzyme can be split into two and hence the assembly of Cas9 for activation can be controlled.

1. A DNA template (an IVT cassette) for making a ribonucleic acid (RNA) transcript having a length of about 20-200 bases, said DNA template comprising
(a) a first deoxyribonucleic acid (DNA) sequence comprising a RNA transcription initiation site;
(b) a polymerase promoter upstream from the RNA transcription initiation site;
(c) a second DNA sequence encoding the RNA transcript having a length of about 20-200 bases disposed downstream of the RNA transcription initiation site; and
(d) a linearization site downstream from the RNA transcription initiation site.

2. The DNA template of claim 1, wherein the template is part of a DNA plasmid.

3.-5. (canceled)

6. The DNA template of claim 1, wherein the DNA template has been linearized.

7. The DNA template of claim 1, further comprising a ribozyme sequence, e.g., downstream from the RNA transcription initiation site and upstream of the linearization site.

8. (canceled)

9. The DNA template of claim 1, further comprising a T7 terminator sequence, e.g., downstream from the RNA transcription initiation site and upstream of the linearization site.

10. The DNA template of claim 1, further comprising a promoter enhancing sequence upstream from the RNA transcription initiation site.

11. The DNA template of claim 1, wherein said RNA transcript having a length of about 20-200 bases comprises a single guide RNA (sgRNA) sequence.

12. (canceled)

13. A double stranded DNA (dsDNA) template for making a ribonucleic acid (RNA) transcript having a length of about 20-200 bases, said dsDNA template comprising
(a) a first DNA sequence comprising an RNA transcription initiation site;
(b) a polymerase promoter upstream from the RNA transcription initiation site,
(c) a second DNA sequence encoding the RNA transcript having a length of about 20-200 bases disposed downstream of the RNA transcription initiation site; and
(d) one or more modified nucleotides at the 5′ end of the antisense strand of the dsDNA template.

14. The dsDNA template of claim 13, comprising a transcriptional enhancer sequence upstream of the polymerase promoter.

15.-17. (canceled)

18. The dsDNA template of claim 13, wherein the linearization site is a restriction endonuclease site.

19. (canceled)

20. The dsDNA template of claim 13, wherein the RNA transcript having a length of about 20-200 bases comprises a sgRNA sequence.

21. (canceled)

22. A partially single stranded DNA (ssDNA) template for making a ribonucleic acid (RNA) transcript having a length of about 20-200 bases, the ssDNA template comprising
(a) a first DNA sequence comprising an RNA transcription initiation site;
(b) a polymerase promoter upstream from the RNA transcription initiation site,
(c) a second DNA sequence encoding the RNA transcript having a length of about 20-200 bases disposed downstream of the RNA transcription initiation site; and
(d) one or more modified nucleotides at the 5′ end of the antisense strand of the dsDNA template.

23. The partially ssDNA template of claim 22, comprising a transcriptional enhancer sequence upstream of the polymerase promoter.

24.-25. (canceled)

26. The partially ssDNA template of claim 22, wherein single stranded DNA is complementary to all or a portion of the polymerase promoter.

27. (canceled)

28. The partially ssDNA template of claim 22, wherein the RNA transcript having a length of about 20-200 bases comprises a sgRNA sequence.

29. (canceled)

30. A method of making a ribonucleic acid (RNA) having a length of about 20-200 bases by in vitro transcription (IVT), comprising the steps of:
(a) obtaining a DNA template of claim 1, and
(b) making the RNA transcript by in vitro transcription.

31. The method of making RNA of claim 30, further comprising the step of amplifying the DNA template using PCR.

32. The method of making RNA of claim 30, further comprising the step of purifying the produced RNA transcript by reverse-phase chromatography.

33. The method of making RNA of claim 30, further comprising the step of testing the purified produced RNA transcript for the presence of immune stimulating moieties by an immunogenicity assay.

34. The method of claim 30, wherein the produced RNA transcript is substantially free of any immune stimulating moieties.

35. The method of claim 30, wherein the produced RNA transcript is substantially free of n+x variants (e.g., where X=1).

36. The method of claim 30, wherein the produced RNA transcript is substantially free of n−x variants (e.g., where X=1).

37. The method of claim 30, wherein the RNA transcript comprises a sgRNA.

38. The method of claim 37, wherein the sgRNA is about 50 bases to 150 bases in length.

39. A composition comprising a ribonucleic acid (RNA) transcript having a length of about 20-200 bases, made by the process of claim 30, wherein:
(a) the composition comprising the RNA transcript is substantially free of immune stimulating moieties, and/or
(b) the composition is substantially free of RNA transcripts having n−1 variants and/or n+1 variants.

40.-43. (canceled)

44. A pharmaceutical composition, comprising the composition of claim 39, and a pharmaceutically acceptable carrier.

45. A composition comprising an IVT-made polynucleotide having a length of about 20-200 bases, wherein the composition is substantially free of immune stimulating moieties and/or is substantially free of n−1 or n+1 variants.

46.-50. (canceled)

51. A cell comprising a composition of claim 39.

52. The cell of claim 51, further comprising an RNA-guided DNA endonuclease enzyme.

53. A method of altering gene expression in a cell, the method comprising introducing into the cell a composition of claim 39.

54. The method of claim 53, further comprising introducing to the cell an RNA-guided DNA endonuclease enzyme.

55.-58. (canceled)

59. A cell, altered by the method of claim 53.

60.-61. (canceled)
